(19) United States
(12) Patent Application Publication
(10) Pub. No.: US 2010/0159513 A1
(43) Pub. Date: Jun. 24, 2010

(54) GENES THAT INCREASE PEPTIDE PRODUCTION

(75) Inventors: Qiong Cheng, Wilmington, DE (US); Kevin Michael Croker, Hockessin, DE (US); Kristin Ruebling-Jass, Wilmington, DE (US); Hong Wang, Kennett Square, PA (US); Jianzhong Zhang, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPNAY, WILMINGTON, DE (US)

(21) Appl. No.: 12/619,790

(22) Filed: Nov. 17, 2009

Related U.S. Application Data
(60) Provisional application No. 61/139,568, filed on Dec. 20, 2008.

(51) Int. Cl. C12P 21/00; C12N 1/21

(52) U.S. Cl. 435/69.7; 435/69.1; 435/252.33

(57) ABSTRACT

Several endogenous genes have been identified in Escherichia coli, the overexpression of which increases recombinant peptide production. Increasing the copy number of aroB, aroK, proB, or crl increases the amount of a recombinant peptide produced by a host cell. Recombinant host cells comprising at least one chimeric genetic construct encoding a peptide of interest and at least one genetic modification that increases recombinant peptide production are provided as well as methods of using such recombinant host cells

### CROSS-REFERENCE TO A RELATED APPLICATION

This application claims the benefit of U.S. Provisional Patent Application No. 61/139,568 filed Dec. 20, 2008, incorporated herein by reference.

### FIELD OF THE INVENTION

The invention relates to the field of molecular biology, microbiology, and recombinant peptide production. More specifically, several endogenous genes have been identified in E. coli that when over-expressed increase recombinant peptide production. Recombinant host cells comprising such modifications as well as a method of producing a peptide of interest using a host cell having one of the present modifications is also provided.

### BACKGROUND OF THE INVENTION

Efficient production of bioactive proteins and peptides is a primary function of the biomedical and the biotechnology industries. In these, bioactive peptides and proteins serve as curative agents for a variety of diseases, such as insulin for diabetes; interferon for viral infections and leukemia; interleukins for diseases of the immune system; and erythropoietin for red blood cell deficiencies. Additionally, large quantities of proteins and peptides are needed for various industrial applications including, but not limited to pulp and paper industries, textiles, food industries, personal care and cosmetics industries, sugar refining, wastewater treatment, production of alcoholic beverages and as catalysts for the generation of new pharmaceuticals.

With the discovery and implementation of combinatorial peptide screening technologies, new applications for small peptides having specific binding affinities have been developed. These technologies include bacterial display (Kemp, D. J.; Proc. Natl. Acad. Sci. USA 78(7): 4520-4524 (1981); yeast display (Chien et al., Proc Natl Acad Sci USA 88(21): 9578-82 (1991)), combinatorial solid phase peptide synthesis (U.S. Pat. No. 5,449,754; U.S. Pat. No. 5,480,971; U.S. Pat. No. 5,585,275 and U.S. Pat. No. 5,639,603), phage display technology (U.S. Pat. No. 5,223,409; U.S. Pat. No. 5,403,484; U.S. Pat. No. 5,571,698; and U.S. Pat. No. 5,837,500), ribosome display (U.S. Pat. No. 5,643,768; U.S. Pat. No. 5,658,754; and U.S. Pat. No. 7,074,557), and mRNA display technology (PROFUSION™; U.S. Pat. No. 6,258,558; U.S. Pat. No. 6,518,018; U.S. Pat. No. 6,281,344; U.S. Pat. No. 6,214,553; U.S. Pat. No. 6,261,804; U.S. Pat. No. 6,207,446; U.S. Pat. No. 6,846,655; U.S. Pat. No. 6,312,927; U.S. Pat. No. 6,602,685; U.S. Pat. No. 6,416,950; U.S. Pat. No. 6,429,300; U.S. Pat. No. 7,078,197; and U.S. Pat. No. 6,436,665).

In the biomedical industry, small peptides are regarded as linkers for the attachment of diagnostic and pharmaceutical agents to surfaces (see U.S. Pat. App. Pub. No. 2003/0185870 to Grinstaff et al., and U.S. Pat. No. 6,620,419 to Linter). In the personal care industry, small peptides serve to attach benefit agents to body surfaces such as hair, skin, nail, and teeth (U.S. Pat. Nos. 7,220,405; 7,309,482; 7,129,326; and 7,285,264; U.S. Pat. App. Pub. Nos. 2002/0098524; 2005/0112692; 2005/0226839; 2007/0196305; 2006/0199206; 2007/0065387; 2008/0107614; 2007/0110686; and 2006/0073111; and Int'l Pat. App. Pub. Nos. WO2008/054746; WO2004/048399, and WO2008/073368)

Commercially-useful peptides may be generated synthetically or isolated from natural sources, which methods may be expensive or time consuming and have limited production capacity. The preferred method of peptide production is via fermentation by recombinant microorganisms engineered to express a protein/peptide of interest.

Although preferable to synthesis or isolation, recombinant peptide production presents obstacles to be overcome to be cost-effective. For example, peptides produced in a cellular environment are susceptible to degradation by native proteases in the cell. Plus, the purification of some peptides may be difficult depending on the nature of the peptide of interest and may result in poor yields.

Mitigating the difficulties associated with recombinant peptide production can involve the use of chimeric genetic constructs encoding chimeric proteins, which include at least one portion that is the desired protein product that is fused to at least one portion comprising a peptide tag. Such chimeric proteins are referred to herein as “fusion proteins”. The peptide tag may be used to assist protein folding, post expression purification and/or protein passage through the cell membrane and to protect the protein from the action of degradative enzymes.

It may be useful to express a peptide in an insoluble form, particularly when the peptide of interest [“POI”] is a small peptide typically soluble under normal physiological conditions and/or subject to proteolytic degradation within the host cell. Production of the peptide in an insoluble form both facilitates simple recovery and protects the peptide from undesirable degradation. Producing the POI in an insoluble form involves recombinantly producing the POI as part of an insoluble fusion protein. In essence, the POI is fused to at least one insoluble peptide tag (also known as a “solubility tag” or “inclusion body tag”). The resulting insoluble fusion protein forms an inclusion body. The fusion protein may include at least one cleavable peptide linker so that the peptide of interest can be subsequently recovered from the fusion protein. The fusion protein may include a plurality of inclusion body tags, cleavable peptide linkers, and regions comprising the peptide of interest.

Another way to up the amount of recovered POI produced by a recombinant host cell is to increase the expression level and/or copy number of the chimeric gene encoding the target peptide, done by: chromosomally integrating multiple copies of the gene; replacing the native promoter with stronger promoters; and/or by using a high copy expression plasmid. The use of high copy plasmids can undesirably burden metabolism of the host cell. And, since the limiting factor in recombinant peptide production may be one or more endogenous components of the host cell's expression system, genetic modifications to one or more endogenous host cell genes may influence the amount of the recombinant peptide produced.

The problem to be solved is to make host cells more efficient in producing POIs. Specifically, this problem relates to identifying endogenous genes within a recombinant microbial host cell, the increased expression of which increases the amount of a POI recombinantly produced within the cell. The problem also relates to a method of producing a POI in a microbial host cell having at least one endogenous gene having increased expression, wherein the increase in expression increases the gene product of a co-expressed chimeric gene. The modified microbial host cells can then be used as a production strain for peptide production.

### SUMMARY OF THE INVENTION

The stated problem has been solved through the over-expression of at least gene selected from the group consisting aroB, aroK, proB, crl, and combinations of these genes, which increases recombinant peptide production in an Escherichia coli production host.

The increase in the expression level of the endogenous gene, which does not encode the peptide of interest, increases the amount of recombinantly-produced POI. Increase in expression of the POI may occur by increasing the copy number of the endogenous gene, the rate of transcription of the endogenous gene, or both.

A method for the production of a peptide of interest is provided comprising:
a) providing a recombinant Escherichia coli host cell comprising
   i) at least one chimeric genetic construct encoding a peptide of interest;
   ii) an over-expressed gene selected from the group consisting of the aroB, aroK, proB, crl, and combination thereof.
b) growing the recombinant E. coli host cell of (a) whereby the peptide of interest is produced;
c) optionally recovering the peptide of interest produced in step (b).

Also described herein are a recombinant Escherichia coli cells comprising:
i) at least one chimeric genetic construct encoding a peptide of interest; and
ii) a genetic modification that increases expression of at least one endogenous gene selected from the group consisting of the aroB, aroK, proB, crl, and a combination thereof.

The recombinant E. coli host cell may further comprise down-regulated expression and/or a disruption in the expression of a chromosomal copy of araBAD operon, slyD gene, or a combination thereof.

The peptide of interest may be a single chain peptide that may range in length from 14 to 600 amino acids. It may be a peptide having strong affinity for at least one target surface, preferably at least one body surface, such as hair, skin, nail, tooth, and tooth pellicle. It may be expressed in the form of a fusion protein comprising the general structure:

IBT-CL-POI

or

POI-CL-IBT

wherein;

IBT means at least one inclusion body tag;

CL means at least one cleavable peptide linker; and

POI means at least one peptide of interest.

### BRIEF DESCRIPTION OF THE FIGURES

FIG. 1. DNA fragments expressed in higher fluorescent clones selected from FACS sorting. In the aroKB clone, the native promoter for aroKB (at position 3517402) was in the opposite orientation as the promoter of Cm^{r} gene on the vector. In the other three cases, the genes on the fragments were expressed in the same orientation as the vector promoter.

FIG. 2. FACS analysis of E. coli hosts containing the isolated expression plasmids. The error bars represent standard deviations of three independent cultures.

FIG. 3 shows the image analysis of the fluorescently labeled peptide band intensity normalized by final OD₆₀₀ of the cultures. The error bars represent standard deviations of samples from three independent cultures.

### BRIEF DESCRIPTION OF THE BIOLOGICAL SEQUENCES

The following sequences conform with 37 C.F.R. 1.821-1.825 (“Requirements for patent applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5 (a-bis), and Section 208 and Annex C of the Administrative Instructions). The symbols and format used for nucleotide and amino acid sequence data comply with the rules set forth in 37 C.F.R. §1.822.

The Sequence Listing is provided herewith electronically.

SEQ ID NO: 1 is the amino acid sequence of a tetracysteine tag that binds to a biarsenical labeling reagent.

SEQ ID NO: 2 is the nucleic acid sequence of peptide expression plasmid pLR199.

SEQ ID NO: 3 is the amino acid sequence of inclusion body tag IBT139.

SEQ ID NO: 4 is the amino acid sequence of a peptide of interest, HC776124.

SEQ ID NO: 5 is the nucleic acid sequence encoding the fusion peptide IBT139-HC776124.

SEQ ID NO: 6 is the amino acid sequence of the fusion peptide IBT139-HC776124.

SEQ ID NOs: 7 and 8 are the nucleic acid sequence of oligonucleotides used to introduce a multiple cloning site (MCS) into plasmid pBHR1.

SEQ ID NO: 9 is the nucleic acid sequence of the coding region for aroB.

SEQ ID NO: 10 is the amino acid sequence of AroB.

SEQ ID NO: 11 the nucleic acid sequence of the coding region for aroK.

SEQ ID NO: 12 is the amino acid sequence of AroK.

SEQ ID NO: 13 is the nucleic acid sequence of the coding region for proB.

SEQ ID NO: 14 is the amino acid sequence of ProB.

SEQ ID NO: 15 is the nucleic acid sequence of the coding region for crl.

SEQ ID NO: 16 is the amino acid sequence of Crl.

SEQ ID NO: 17 is the nucleic acid sequence of the coding region for mreC.

SEQ ID NO: 18 is the amino acid sequence of MreC.

SEQ ID NO: 19 is the nucleic acid sequence of the araB promoter.

SEQ ID NO: 20 is the nucleic acid sequence of the coding sequence for the slyD gene in Escherichia coli strain K-12 substrain MG1655.

SEQ ID NO: 21 is the amino acid sequence of the SlyD protein in Escherichia coli strain K-12 substrain MG1655.

SEQ ID NOs: 22-33 are nucleic acid sequences of various primers and probes used in Example 6.

SEQ ID NO: 34 is the amino acid sequence of the Caspase 3 cleavage site.

SEQ ID NOs: 35-281 are the amino acid sequences of various body surface-binding peptides are shown in Table A. SEQ ID NOs: 35-191 bind to hair, SEQ ID NOs: 187-239 bind to skin, SEQ ID NOs: 240-241 bind to nail, and SEQ ID NOs: 242-281 bind to a tooth surface, wherein SEQ ID NOs: 242-261 bind to tooth pellicle and SEQ ID NOs: 262-281 bind to tooth enamel.

SEQ ID NOs: 282-340 are the amino acid sequences of polymer-binding peptides as shown in Table A.

SEQ ID NOs: 341-344 are the amino acid sequences of cellulose acetate-binding peptides.

SEQ ID NOs: 345-399 are the amino acid sequences of pigment-binding peptides as shown in Table A.

SEQ ID NOs: 400-411 are the amino acid sequences of print media-binding peptides as shown in Table A.

SEQ IS NOs: 412-426 are the amino acid sequence of clay-binding peptides.

SEQ ID NOs: 427-452 are calcium carbonate-binding peptides.

SEQ ID NOs: 453-481 are the amino acid sequences of various antimicrobial peptides (U.S. Pat. No. 7,427,656).

### DETAILED DESCRIPTION

Several endogenous genes in E. coli are described herein whose overexpression increases recombinant peptide production. Increasing the expression level of aroB, aroK, proB, or crl increases the amount of a recombinant peptide produced by the E. coli cell. Recombinant host cells comprising at least one chimeric genetic construct encoding a peptide of interest and at least one genetic modification that increases expression of at least one of the identified genes is provided as well as a method of recombinantly producing a peptide of interest using such host cells.

The following definitions are used herein and should inform the interpretation of the claims and the specification. Unless otherwise noted, all U.S. patents and U.S. patent applications referenced herein are incorporated by reference in their entirety.

As used herein, the indefinite articles “a” and “an” preceding an element or component of the invention are intended to be nonrestrictive regarding the number of instances (i.e., occurrences) of the element or component. Therefore “a” or “an” should be read to include one or at least one, and the singular word form of the element or component also includes the plural unless the number is obviously meant to be singular.

As used herein, the term “comprising” refers to the presence of the stated features, integers, steps, or components as referred to in the claims, but that it does not preclude the presence or addition of one or more other features, integers, steps, components or groups thereof. This means a composition, a mixture, a process, a method, an article, or an apparatus that comprises a list of elements is not limited to only those elements but may include others not expressly listed or inherent to it. As used herein, “or” refers to an inclusive and an exclusive “or”. For example, a condition A or B is satisfied by any one of the following: A is true (or present) and B is false (or not present), A is false (or not present) and B is true (or present), and both A and B are true (or present).

As used herein, the term “about” refers to modifying the quantity of an ingredient or reactant of the invention or employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

As used herein, the term “invention” or “present invention” is a non-limiting term and is not intended to refer to any single embodiment of the particular invention but encompasses all possible embodiments as described in the specification and the recited in the claims.

As used herein, the terms “polypeptide” and “peptide” are used interchangeably to refer to a polymer of two or more amino acids joined together by a peptide bond. In one aspect, this term also includes post expression modifications of the polypeptide, for example, glycosylations, acetylations, phosphorylations and the like. Included within the definition are, for example, peptides containing one or more analogues of an amino acid or labeled amino acids and peptidomimetics. In one embodiment, the peptides are comprised of L-amino acids.

As used herein, the terms “peptide of interest”, “gene product”, “target gene product”, and “target coding region gene product” refer to the desired peptide/protein product encoded by the recombinantly expressed foreign gene. Peptide of interest may abbreviated “POI”. The peptide of interest may include any peptide/protein product including, but not limited to proteins, fusion proteins, enzymes, peptides, polypeptides, and oligopeptides. In one embodiment, the peptide of interest ranges in size from 14 to 600 amino acids in length.

As used herein, the terms “bioactive” or “peptide of interest activity” refer to the activity or characteristic associated with the peptide and/or protein of interest. The bioactive peptides may be used as, for example, curative agents for diseases (e.g., insulin, interferon, interleukins, anti-angiogenic peptides (U.S. Pat. No. 6,815,426); polypeptides that bind to defined cellular targets such as receptors, channels, lipids, cytosolic proteins, and membrane proteins; peptides having antimicrobial activity; peptides having an affinity for a particular material (e.g., hair-binding polypeptides, skin-binding polypeptides, nail-binding polypeptides, cellulose-binding polypeptides, polymer-binding polypeptides, clay-binding polypeptides, silica-binding polypeptides, carbon nanotube-binding polypeptides and peptides that have an affinity for particular animal or plant tissues) for targeted delivery of benefit agents.

As used herein, the “benefit agent” refers to a molecule that imparts a desired functionality or benefit when applied or coupled to a target surface. The present peptide reagents may be used to couple a benefit agent to a target surface, such as a body surface. In one embodiment, the peptide reagent is used to couple a benefit agent to a body surface by forming a complex between the peptide reagent, the benefit agent, and the body surface. In one embodiment, the peptide reagent is applied to the body surface prior to the application of the benefit agent (i.e., a sequential application). The benefit agent may be a peptide or the peptide reagent (e.g. condition peptides or antimicrobial peptides) or may be one or more molecules bound to (covalently or non-covalently), or associated with, a peptide reagent having affinity for a target surface. The benefit agent may be a particulate benefit agent. In one embodiment, the term “particulate benefit agent’ is a general term relating to a particulate substance, which when applied to a body surface provides a cosmetic or prophylactic effect. Particulate benefit agents typically include pigments, particulate conditioners, inorganic sunscreens and the like along with other particulate substances commonly used in the personal care industry.

As used herein, the term “MB₅₀” refers to the concentration of the binding peptide that gives a signal that is 50% of the maximum signal obtained in an ELISA-based binding assay (see Example 9 of U.S. Pat. App. Pub. No. 2005-0226839; hereby incorporated by reference). The MB₅₀ provides an indication of the strength of the binding interaction or affinity of the components of the complex. The lower the value of MB₅₀, the stronger the interaction of the peptide with its corresponding substrate.

As used herein, the terms “binding affinity” or “affinity” refers to the strength of the interaction of a binding peptide (e.g. target surface-binding peptides, target surface-binding domains, and peptide reagents) with its respective substrate. The binding affinity may be reported in terms of the MB₅₀ value as determined in an ELISA-based binding assay or as a K_{D} (equilibrium dissociation constant) value, which may be deduced using surface plasmon resonance (SPR). The lower the value of MB₅₀ or K_{D}, the stronger affinity of the peptide interacting with its corresponding substrate.

As used herein, the term “strong affinity” refers to a binding affinity, as measured as an MB₅₀ or K_{D} value, of 10⁻⁴ M or less, preferably 10⁻⁵ M or less, preferably less than 10⁻⁸ M, more preferably less than 10⁻⁷ M, more preferably less than 10⁻⁸ M, even more preferably less than 10⁻⁹ M, and most preferably less than 10⁻¹⁰ M.

As used herein, the term “target surface-binding peptide” refers to a single chain peptide having strong affinity (defined as having a K_{D} value less than 10⁻⁴ M or an MB₅₀ value of less than 10⁻⁴) for a target surface. The peptide of interest may be a single target surface-binding peptide ranging in size from 7 to 60 amino acids in length, or may be a single chain, peptide-based reagent comprising one or more target surface-binding peptides, wherein the length of the peptide-based reagent ranges from 14 to 600 amino acids in length. The target surface-binding peptide may be a body surface-binding peptide.

As used herein, the term “body surface-binding peptide” refers to a peptide having strong affinity for a body surface. Examples of body surfaces include, but are not limited to hair, skin, nail, and tooth. The body surface-binding peptides are typically used to couple a personal or health care benefit agent to the body surface. These agents include colorants, conditioners, and antimicrobials, to name a few. Means to identify suitable body-surface binding peptides are well known in the art and may include biopanning techniques such as phage display, bacterial display, yeast display, ribosome display, and mRNA-display, etc. The body surface-binding peptide may also be empirically-generated.

As used herein, “HBP” refers to and is an abbreviation for hair-binding peptide. As used herein, the term “hair-binding peptide” refers to a peptide that binds with high affinity to hair. Examples of hair-binding peptides have been reported (U.S. patent application Ser. No. 11/074,473 to Huang et al.; Int'l Pat. App. Pub. No. WO 200179479; U.S. Pat. App. Pub. No. 2002/0098524 to Murray et al.; U.S. Pat. App. Pub. No. 2003/0152976 to Janssen et al.; Int'l Pat. App. Pub. No. WO 2004048399; U.S. application Ser. No. 11/512,910, and U.S. patent application Ser. No. 11/696,380). Examples of hair-binding peptides are provided as SEQ ID NOs: 35-191. The hair-binding peptides may be from about 7 amino acids to about 60 amino acids, more preferably, from about 7 amino acids to about 25 amino acids, most preferably from about 7 to about 20 amino acids in length.

As used herein, “SBP” means skin-binding peptide. As used herein, the term “skin-binding peptide” refers to a peptide sequence that binds with high affinity to skin. Examples of skin-binding peptides have also been reported (U.S. patent application Ser. No. 11/069,858 to Buseman-Williams; Int'l Pat. App. Pub. No. WO 2004/000257; and U.S. patent application Ser. No. 11/696,380). Skin as used herein as a body surface will generally comprise a layer of epithelial cells and may additionally comprise a layer of endothelial cells. Examples of skin-binding peptides are provided as SEQ ID NOs: 157-239. The skin-binding peptides may be from about 7 amino acids to about 60 amino acids, more preferably, from about 7 amino acids to about 25 amino acids, most preferably from about 7 to about 20 amino acids in length.

As used herein, “NBP” refers to and is an abbreviation for nail-binding peptide. As used herein, the term “nail-binding peptide” refers to a peptide that binds with high affinity to nail. Examples of nail-binding peptides have been reported (U.S. patent application Ser. No. 11/696,380). Examples of nail-binding peptides are provided as SEQ ID NOs: 240-241. The nail-binding peptides may be from about 7 amino acids to about 60 amino acids, more preferably, from about 7 amino acids to about 25 amino acids, most preferably from about 7 to about 20 amino acids in length.

As used herein, “TBP” refers to and is an abbreviation for tooth-binding peptide. A tooth-binding peptide is a peptide that binds with high affinity to a mammalian or human tooth surface. As used herein, the term “tooth-binding peptide” will refer to a peptide that binds to tooth enamel or tooth pellicle. In one embodiment, the tooth-binding peptides may be from about 7 amino acids to about 60 amino acids in length, more preferably, from about 7 amino acids to about 25 amino acids in length, most preferably from about 7 to about 20 amino acids in length. In a preferred embodiment, the tooth-binding peptides are combinatorially-generated peptides. Examples of tooth-binding peptides having been disclosed in co-pending and co-owned U.S. application Ser. No. 11/877,692 and are provided as SEQ ID NOs: 242-281.

As used herein, the term “tooth surface” refers to a surface comprised of tooth enamel (typically exposed after professional cleaning or polishing) or tooth pellicle (an acquired surface comprising salivary glycoproteins). Hydroxyapatite can be coated with salivary glycoproteins to mimic a natural tooth pellicle surface (tooth enamel is predominantly comprised of hydroxyapatite).

As used herein, the terms “pellicle” and “tooth pellicle” refer to the thin film (typically ranging from about 1 μm to about 200 μm thick) derived from salivary glycoproteins which forms over the surface of the tooth crown. Daily tooth brushing tends to remove only a portion of the pellicle surface while abrasive tooth cleaning and/or polishing (typically by a dental professional) will expose more of the tooth enamel surface.

As used herein, the terms “enamel” and “tooth enamel” refer to the highly mineralized tissue which forms the outer layer of the tooth, which is composed primarily of crystalline calcium phosphate (i.e. hydroxyapatite; Ca₅(PO₄)₃OH), water and some organic material. The tooth surface may be selected from the group consisting of tooth enamel and tooth pellicle.

As used herein, the terms “peptide linker”, “linker” and “peptide spacer” refer to a peptide used to link together two or more target surface-binding peptides.

As used herein, the terms “bridge”, “peptide bridge”, and “bridging element” refer to a linear peptide used to couple a target-surface binding domain (“target surface-binding hand”) to a peptide domain coupled to the surface of particulate benefit agent (i.e. covalent or non-covalent coupling). The peptide bridge may range in size from 1 to 60 amino acids in length, preferably 6 to 40 amino acids in length.

As used herein, the terms “coupling” and “coupled” refer to any chemical association and include both covalent and non-covalent interactions. Coupling can mean a covalent interaction or a non-covalent interaction.

As used herein, the terms “hand”, “target surface hand”, and “target surface-binding domain” refer to a single chain peptide comprising of at least two target surface-binding peptides linked together by at least one peptide linker. The target surface-binding peptides may be biopanned from a random peptide library using a method selected from the group consisting of phage display, bacterial display, yeast display, ribosome display, and mRNA-display. The target-surface binding hand may comprise two target surface-binding peptides linked together by a peptide linker.

As used herein, the term “peptide-based reagent” or “peptide reagent” refers to a single chain peptide comprising at least one target surface-binding domain having strong affinity for a target surface.

As used herein, the term “body surface-binding hand” or “body surface-binding domain” refer to a single chain peptide comprising two or more body surface-binding peptides (BSBPs) linked together by at least one peptide linker. The body surface-binding domain may comprise two body surface-binding peptides linked together by a peptide linker.

As used herein, the term “benefit agent-binding hand” or “benefit agent-binding domain” refer to a single chain peptide domain comprising two or more benefit agent-binding peptides (BABPs) coupled together by at least one peptide linker. The benefit agent-binding domain may comprise two benefit agent-binding peptides linked together by a peptide linker.

As used herein, the term “inclusion body tag”, abbreviated as “IBT”, refers to a polypeptide that facilitates formation of inclusion bodies when fused to a peptide of interest. The peptide of interest is typically soluble within the host cell and/or host cell lysate when not fused to an inclusion body tag. Fusion of the peptide of interest to the inclusion body tag produces a fusion protein that agglomerates into intracellular bodies, also called inclusion bodies, within the host cell. The fusion protein comprises a portion having an inclusion body tag and a peptide/protein of interest. The polypeptide/protein of interest may be separated from the inclusion body tags using cleavable peptide linker elements (See U.S. patent application Ser. Nos. 11/641,936, 11/641,273, and 11/782,836).

As used herein, the terms “cleavable linker element” and “cleavable peptide linker” are used interchangeably and refer to cleavable peptide segments typically incorporated between an inclusion body tag and the peptide of interest. After the inclusion bodies are separated and/or partially-purified or purified from the cell lysate, the cleavable linker element can be cleaved chemically and/or enzymatically to separate the inclusion body tag from the peptide of interest. The fusion peptide may also include a plurality of regions encoding one or more peptides of interest separated by one or more cleavable peptide linkers. The peptide of interest can then be isolated from the inclusion body tag, if necessary.

The inclusion body tag(s) and the peptide of interest may exhibit different solubilities in a defined medium, typically aqueous, thereby facilitating separation of the inclusion body tag from the peptide of interest. Preferably, the inclusion body tag is insoluble in an aqueous solution while the protein/peptide of interest is appreciably soluble in an aqueous solution. The pH, temperature, and/or ionic strength of the aqueous solution can be adjusted to facilitate recovery of the peptide of interest. The differential solubility between the inclusion body tag and the peptide of interest may occur in an aqueous solution having a pH of 5 to 10 and a temperature range of 15° C. to 50° C. The cleavable peptide linker may be from 1 to about 50 amino acids in length, preferably from 1 to about 20 amino acids in length. An example of an enzymatically cleavable peptide linker is provided by SEQ ID NO: 34 (Caspase-3 cleavage sequence). The cleavable linker may be an acid cleavable aspartic acid—proline dipeptide (D-P) moiety. The cleavable peptide linkers may be incorporated into the fusion proteins using any number of techniques well known in the art.

As used herein, the term “genetic construct” refers to a series of contiguous nucleic acids useful for modulating the genotype or phenotype of an organism. Non-limiting examples of genetic constructs include but are not limited to a nucleic acid molecule, and open reading frame, a gene, an expression cassette, a vector, a plasmid and the like.

As used herein, the terms “fusion protein” and “fusion peptide” are interchangeable and refer to a polymer of amino acids (peptide, oligopeptide, polypeptide, or protein) comprising at least two portions, each portion comprising a distinct function. A first portion of the fusion peptide may comprise at least one inclusion body tag and a second portion of the fusion peptide may comprise at least one peptide of interest. The fusion protein may additionally include at least one cleavable peptide linker that facilitates chemical and/or enzymatic cleavage and separation of the inclusion body tag(s) and the peptide(s) of interest.

As used herein, the term “polymer-binding peptide” refers to peptide sequences that bind with high affinity to a specified polymer (U.S. Pat. App. 11/516,362). Examples of polymer-binding peptides are provided as SEQ ID NOs: 282-341.

As used herein, the term “pigment” refers to an insoluble colorant. A wide variety of organic and inorganic pigments alone or in combination may be used in the methods and host cells described herein.

As used herein, the terms “iron oxide-based pigment” and “iron oxide pigment” refers to a pigment particle comprised primarily of an iron oxide. Iron oxide pigments may vary in color (red, yellow, brown, and black tones) due to minor impurities and/or the size of the pigment particle. In one embodiment, the iron oxide pigment is a cosmetically acceptable iron oxide pigment. Cosmetically acceptable iron oxide pigments are commercially available from various companies, such as Sensient Technologies Corp, Milwaukee, Wis. The iron oxide is selected from the group consisting of ferric oxide (Fe₂O₃), ferrous ferric oxide (Fe₃O₄), and mixtures of Fe₂O₃ and Fe₃O₄. The iron oxide may be ferric oxide Fe₂O₃. The iron oxide pigment may be at least partially coated with silica.

As used herein, the term “pigment-binding peptide” refers to a peptide that binds with high affinity to a pigment particle. Examples of pigment-binding peptides are provided in Table A as SEQ ID NOs 345-399. SEQ ID NOs: 345-348 bind to carbon black, SEQ ID NOs: 349-357 bind to CROMOPHTAL® yellow, SEQ ID NOs: 358-360 bind to SUNFAST® magenta, SEQ ID NOs: 357 and 361-369 bind to SUNFAST® blue, and SEQ ID NOs: 370-399 bind to iron oxide-based pigments.

As used herein, an “antimicrobial peptide” is a peptide having the ability to kill microbial cell populations (U.S. Pat. No. 7,427,656). Examples of antimicrobial peptides are provided as SEQ ID NOs: 453-481.

As used herein, the term “print medium-binding peptide” will refer to a peptide that binds to a printer medium such as cotton, cellulose, paper, and cotton/polyester blends. Examples of print media-binding peptides are provided as SEQ ID NOs: 400-411.

As used herein, “clay-binding peptide” refers to a peptide that binds with strong affinity to clay (U.S. patent application Ser. No. 11/696,380). Examples of clay-binding peptides are provided as SEQ ID NOs: 412-426.

As used herein, “calcium carbonate-binding peptide” refers to a peptide that binds with strong affinity of calcium carbonate (U.S. Pat. App. No. 11/828,539). Examples of calcium carbonate-binding peptides are provided as SEQ ID NOs: 427-452.

As used herein, the term “operably-linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). In a further embodiment, the definition of “operably linked” may also be extended to describe the products of chimeric genes. As such, “operably-linked” may also refer to the linking of two or more target surface-binding peptides by at least one peptide linker.

As used herein, the term “effective amount” refers to that amount of a specified material or combination of materials, such as a least one peptide-based reagent and the amount of at least one benefit agent, incorporated into a composition to achieve the desired effect.

As used herein, the term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Miscellaneous (or as defined herein) | Xaa | X |

As used herein, the term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

As used herein, the term “peptide-based” refers to an interfacial material comprised of a compound pertaining to or having the nature or the composition of the peptide class. Interfacial refers to the quality of the peptide-based material described herein as connecting one material to another.

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

### Methods of Altering Expression Levels

Methods of altering the expression of a gene are well known in the art. As used herein, “altered expression” will refer to a genetic change introduced to a host cell that increases (overexpression) or decreases the amount of the resulting gene product, such as a peptide or protein, when compared to the non-modified cell under similar conditions. Overexpression of a gene is accomplished typically by increasing the copy number of the gene or by incorporating a regulatory element that increases the level of transcription, such as a stronger promoter.

Conversely, one may need to “decrease” or “down-regulate” expression of one or more endogenous genes whose gene product(s) may adversely impact the amount of the peptide of interest produced. There are many modifications that can be introduced into a host cell to decrease the expression of a gene including, but not limited to, decreasing the copy number of the gene if there are multiple copies); substituting the endogenous regulatory element with a regulatory element characterized by weaker transcription activity (such as a weaker promoter); introducing mutations to the gene that results lower yield of the functional gene product (including point mutations, additions, or deletions); antisense expression; and genetic modifications that disrupt the production of the functional gene product, such as genetic “knock-outs”. Assuming the gene targeted for disruption is not essential, a gene can be disrupted by any number of techniques including, but not limited to the introduction of insertions, partial or complete deletions or mutations to the gene that eliminate production of the functional gene product. Given the number of whole genome sequences publicly available, one may use a targeted approach to disrupt the gene of interest. Conversely, random mutagenesis may also be used to down regulate or disruption expression of one or more genes.

As illustrated herein, the E. coli strain further comprises a knock-out to the endogenous araBAD operon, a pBAD expression vector used to drive expression of the chimeric gene encoding the fusion peptide, and a knock-out to the slyD gene to remove possible binding between the LUMIO™ biarsenical labeling reagent and cysteine rich sequences in slyD. The E. coli production host may comprise a genetic modification that results in decreased expression and/or a disruption in the endogenous araBAD operon, the slyD gene, or a combination of these.

The examples illustrate, a microbial host cell comprising a chimeric gene encoding a peptide of interest was prepared and expressed from a first plasmid. A library of chromosomal fragments was also prepared and incorporated into a second plasmid. The chromosomal fragments were co-expressed with the chimeric gene encoding the peptide of interest, in this instance, expressed on a compatible plasmid. Interestingly, overexpression by increasing copy number of several endogenous genes resulted in an increased amount of the peptide of interest relative to a control, that is empty vector, under similar expression conditions. Although an increase in copy number was exemplified, one of skill in the art will recognize that other modifications, such as substituting a stronger promoter, may also be used to increase the amount of the gene product encoded by the endogenous gene.

Overexpression of Endogenous Escherichia coli Genes

Several genes were initially identified from the fragment library that were associated with an increase in fluorescence. The increase in fluorescence was initially attributed to an increase in the amount of produced fusion peptide. Further analysis confirmed that the amount of peptide of interest produced increased relative to the control under identical conditions for all of the overexpressed genes except mreC. The higher fluorescence observed by FACS when overexpressing mreC may be associated with defective cell division, as it was reported that introduction of the mre genes on multicopy plasmid leads to filamentation phenotype (Lee, et al., (2003), Current Microbiology 47:146).

To confirm that mRNA transcript levels of the endogenous genes in these strains were increased, real time reverse transcription-PCR (RT-PCR) analysis was performed. Overexpression of aroB, aroK, crl, or proB increased the amount of the fusion peptide (i.e. the model “peptide of interest”). Consequently, is it believed that overexpressing combinations of these genes will also increase the amount of fusion peptide produced.

AroB and AroK

The aroB gene encodes 3-dehydroquinate synthase (EC 4.2.3.4; AroB) which catalyzes the conversion of 3-deoxy-D-arabino-heptulosonate-7-phosphate to 3-dehydroquinate (Frost, J W et al, 1984, Biochemistry 23(19):4470-4475). The aroK gene encodes shikimate kinase I (E.C. 2.7.1.71; AroK), which catalyzes the conversion of shikimate to shikimate-3-phosphate. The aroK and aroB genes are involved in the synthesis of chorismate, the common precursor for aromatic amino acids biosynthesis. The aroK gene is upstream of aroB genes and the aroKB genes are organized in an operon (Lobner-olesen and Marinus, (1992) J. Bacteriol. 174(2):425-529).

The term “aromatic amino acid biosynthetic pathway” refers to a ubiquitous enzymatic pathway found in many microorganisms responsible for synthesis of tryptophan, phenylalanine and tyrosine. The aromatic amino acid biosynthetic pathway includes the common pathway leading to the synthesis of the chorismate precursor and the branched pathways unique for each of the three aromatic amino acids. The common pathway for chorismate biosynthesis comprises the enzymes encoded by the genes aroF, aroG, aroH, aroB, aroD, aroE, aroL, aroK, aroA, aroC. The conversion from chorismate to tryptophan involves enzymes encoded by the genes trpE, trpD, trpC, trpB and trpA. The conversion from chorismate to phenylalanine involves enzymes encoded by the genes pheA and tyrB. The conversion from chorismate to tyrosine involves enzymes encoded by the genes tyrA and tyrB. U.S. Pat. App. Pub. No. 2008/0102499 describes the genes involved in aromatic amino acid biosynthesis and an enhanced tyrosine over-producing host cell.

The aroB coding sequence is provided as SEQ ID NO: 9. The amino acid sequence of the AroB protein is provided as SEQ ID NO: 10. The aroK coding sequence is provided as SEQ ID NO: 11. The amino acid sequence of the AroK protein is provided as SEQ ID NO: 12.

ProB

The proB gene encodes γ-glutamyl kinase (EC 2.7.2.11) (ProB), which catalyses the conversion of L-glutamate to L-glutamate-5-phosphate (Smith C J, et al, 1984, J. Bacteriol. 157(2):545-551). This is the first step in proline biosynthesis. The proA gene encodes the glutamate-5-semialdehyde dehydrogenase (EC 1.2.1.41), which catalyzes the conversion of L-glutamate-5-phosphate to L-glutamate-γ-semialdehyde (Hayzer, D J, et al, 1982, Eur. J. Biochem. 121(3):561-565). The L-glutamate-γ-semialdehyde is spontaneously cyclized to 1-pyrroline-5-carboxylate, which is then converted to L-proline by the pyrroline-5-carboxylate reductase (encoded by proC). The proB gene is upstream of proA gene and the proBA genes are organized in an operon. The enzymes encoded by proBA genes form a complex. The proB coding sequence is provided as SEQ ID NO: 13. The amino acid sequence of the ProB protein is provided as SEQ ID NO: 14.

Crl

The crl gene encodes a transcriptional regulator that regulates the activity and abundance of the RNA polymerase component sigma S. Crl stimulates rpoS activity during stationary phase (Pratt. L A, et al, 1998, Mol. Microbiol. 29(5):1225-1236). Crl acts by tilting the competition between RNA polymerase sigma S factor and the RNA polymerase sigma D (sima 70) factor for binding to the core enzyme of RNA polymerase towards sigma S (Typas et al., (2007) EMBO J., 6(6):1569-1578). The crl coding sequence is provided as SEQ ID NO: 15. The amino acid sequence of the Crl protein is provided as SEQ ID NO: 16.

MreC

The mreBCD genes are involved in peptidoglycan biosynthesis and are responsible for rod shape of E. coli cells (Wachi M, et al, 1987, J. Bacteriol. 169(11):4935-4940; Wachi M, et al, 1989, J. Bacteriol. 171(12):6511-6515). Overexpression of mre genes inhibits cell division and leads to filamentous cell morphology (Kruse T, et al, 2003, EMBO J. 22(19):5283-5292; Lee, J.-C., et al, 2002, Current Microbiol. 47:146-152). Overexpression of mre genes resulted in an increase in fluorescence. However, further analysis (Example 4) indicated that the amount of fusion peptide produced, i.e. the peptide of interest, did not significantly increase. The mreC coding sequence is provided as SEQ ID NO: 17. The amino acid sequence of the MreC protein is provided as SEQ ID NO: 18.

The present modifications may be extended to other microbial host cells comprising substantially-similar structural homologs of the present genes. Means to identify substantially similar biological molecules are well known in the art (e.g. sequence alignment protocols, nucleic acid hybridizations, presence of a conserved signature motifs, etc.). In one aspect, the product of the overexpressed endogenous gene in the present process comprises a substantially similar polypeptide having at least 40%, preferably at least 50%, more preferably at least 60%, even more preferable at least 70%, even more preferably at least 80%, yet even more preferable at least 90%, and most preferably at least 95% amino acid identity to a sequence selected from the group consisting of SEQ ID NOs: 10, 12, 14, or 16.

Nucleic acid hybridization may also be used to identify substantially similar nucleic acid sequences. The present nucleic acid molecules may be used to identify genes encoding substantially similar polypeptides/proteins. Nucleic acid hybridization may be conducted under stringent conditions.

Each of the proposed modifications is well within the routine skill in the art (see Sambrook and Russell, supra). Moreover, the skilled artisan recognizes that substantially similar sequences are encompassed by the present invention. In one embodiment, substantially similar sequences are defined by their ability to hybridize, under the following stringent conditions (0.1×SSC, 0.1% SDS, 65° C. and washed with 2×SSC, 0.1% SDS followed by 0.1×SSC, 0.1% SDS, 65° C.) with a sequence selected from the group consisting of SEQ ID NOs. 9, 11, 13, and 15. Furthermore, the present modifications illustrated in Escherichia coli K-12 substrain MG1655 should apply to other E. coli strains, especially derivatives of strain K-12.

### Peptide of Interest

The function of the peptide of interest is not limited by the present method and may include, but is not limited to bioactive molecules that act as curative agents for diseases, such as insulin, interferon, interleukins, peptide hormones, anti-angiogenic peptides, and peptides that bind to and affect defined cellular targets such as receptors, channels, lipids, cytosolic proteins, and membrane proteins (see U.S. Pat. No. 6,696,089); peptides having an affinity for a particular material, such as biological tissues, biological molecules, hair-binding peptides (see U.S. patent application Ser. No. 11/074,473; Int'l Pat. App. No. WO 0179479; U.S. Pat. App. Pub. No. 2002/0098524; U.S. Pat. App. Pub. No. 2003/0152976; Int'l Pat. App. No. WO 04048399; U.S. Pat. App. Pub. No 2007/0067924; and U.S. Pat. App. Pub. No. 2007/0249805), skin-binding peptides (see U.S. Pat. No. 7,309,482; Int'l Pat. App. No. WO 2004/000257; and U.S. Pat. App. Pub. No. 2007/0249805), nail-binding peptides (see U.S. Pat. App. Pub. No. 2007/0249805), cellulose-binding peptides, polymer-binding peptides (see U.S. Pat. App. Pub. Nos. 2007/0141629, 2007/0264720, 2008/0207872, 2007/0141628, and 2007/0261775), clay-binding peptides, silica-binding peptides, and carbon nanotube binding peptides) for targeted delivery of at least one benefit agent (see U.S. patent application Ser. No. 10/935,642; U.S. patent application Ser. No. 11/074,473; and U.S. Pat. App. Pub. No. 2007/0249805).

### Single Chain Peptides Having Affinity for a Target Surface

Proteinaceous materials having strong affinity for a body surface have been used for targeted delivery of one or more personal care benefit agents. However, many of these materials used for targeted delivery are comprised or derived from immunoglobulins or immunoglobulin fragments (antibodies, antibody fragments, F_{ab}, single-chain variable fragments (scFv), and Camilidae V_{HH}) having affinity for the target surface. For example, Horikoshi et al. in JP 08104614 and Igarashi et al. in U.S. Pat. No. 5,597,386 describe hair coloring agents that consist of an anti-keratin antibody covalently attached to a dye or pigment. The antibody binds to the hair, thereby enhancing the binding of the hair coloring agent to the hair. Similarly, Kizawa et al. in JP Pat. App. Pub. No. 09003100 describe an antibody that recognizes the surface layer of hair and its use to treat hair. A hair coloring agent consisting of that anti-hair antibody coupled to colored latex particles is also described. The use of antibodies to enhance the binding of dyes to the hair is effective in increasing the durability of the hair coloring, but the antibodies are difficult and expensive to produce. Terada et al. in JP Pat. App. Pub. No. 2002363026 describe the use of conjugates consisting of single-chain antibodies, preferably anti-keratin, coupled to dyes, ligands, and cosmetic agents for skin and hair care compositions. Although single-chain antibodies may be prepared using genetic engineering techniques, these molecules are expensive to prepare and may not be suitable for use in commercial personal care products due to their conserved structure (i.e. immunoglobulin folds) and large size.

Non-immunoglobulin derived scaffold proteins have also been developed for targeted delivery of benefit agents to a target surface, such as delivery of cosmetic agents to keratin-containing materials (See Binz, H. et al. (2005) Nature Biotechnology 23, 1257-1268 for a review of various proteins used in scaffold-assisted binding). Findlay in Int'l App. Pub. No. WO 00/048558 describes the use of calycin-like scaffold proteins, such as β-lactoglobulin, which contain a binding domain for a cosmetic agent and another binding domain that binds to at least a part of the surface of a hair fiber or skin surface, for conditioners, dyes, and perfumes. Houtzager et al. in Int'l App. Pub. No. WO 03/050283 and U.S. Pat. App. Pub. No.

2006/0140889 also describe affinity proteins having a defined core scaffold structure for controlled application of cosmetic substances. As with immunoglobulin-like proteins, these large scaffold protein are somewhat limited by the requirement to maintain the underlying core structure for effective binding and are expensive to produce.

Preferably, the peptide of interest is a single chain peptide having strong affinity for at least one target surface and ranges in length from about 14 to about 600 amino acids and does not comprise an immunoglobulin fold or scaffold support.

Target surface-binding peptides having strong affinity for a target surface can be identified and isolated from peptide libraries using any number of biopanning techniques well known to those skilled in the art including, but not limited to bacterial display (Kemp, D. J.; Proc. Natl. Acad. Sci. USA 78(7): 4520-4524 (1981); yeast display (Chien et al., Proc Natl Acad Sci USA 88(21): 9578-82 (1991)), combinatorial solid phase peptide synthesis (U.S. Pat. No. 5,449,754; U.S. Pat. No. 5,480,971; U.S. Pat. No. 5,585,275 and U.S. Pat. No. 5,639,603), phage display (U.S. Pat. No. 5,223,409; U.S. Pat. No. 5,403,484; U.S. Pat. No. 5,571,698; and U.S. Pat. No. 5,837,500), ribosome display (U.S. Pat. No. 5,643,768; U.S. Pat. No. 5,658,754; and U.S. Pat. No. 7,074,557), and mRNA display technology (PROFUSION™; U.S. Pat. No. 6,258,558; U.S. Pat. No. 6,518,018; U.S. Pat. No. 6,281,344; U.S. Pat. No. 6,214,553; U.S. Pat. No. 6,261,804; U.S. Pat. No. 6,207,446; U.S. Pat. No. 6,846,655; U.S. Pat. No. 6,312,927; U.S. Pat. No. 6,602,685; U.S. Pat. No. 6,416,950; U.S. Pat. No. 6,429,300; U.S. Pat. No. 7,078,197; and U.S. Pat. No. 6,436,665). Techniques to generate random peptide libraries are described in iDani, M., J. of Receptor & Signal Transduction Res., 21(4):447-468 (2001). Phage display libraries are available commercially from companies such as New England BioLabs (Beverly, Mass.).

The biopanned target surface-binding peptides are typically about 7 to about 60 amino acids in length and often have a binding affinity as measured by an MB₅₀ or K_{D} of 10⁻⁴ M or less for the surface of the target material.

Single chain peptide-based reagents have been developed that can be used to couple benefit agents to a target surface. Examples of target surfaces include, but not are limited to body surfaces such as hair, skin, nail, and teeth (U.S. Pat. Nos. 7,220,405; 7,309,482; and 7,285,264; U.S. Pat. App. Pub. Nos. US2005-0226839; US2007-0196305; US2006-0199206; US2007-0065387; US2008-0107614; US2007-0110686; and US2006-0073111; and Int'l Pat. App. Pub. Nos. WO2008/054746; WO2004/048399, and WO2008/073368) as well as other surfaces such as pigments and miscellaneous print media (U.S. Pat. App. Pub. No. 2005-0054752), and various polymers such as polymethylmethacrylate (U.S. Pat. App. Pub. No. 2007-0265431), polypropylene (U.S. Pat. App. Pub. No. 2007-0264720), nylon (U.S. Pat. App. Pub. Nos. 2007-0141629 and 2003-0185870), polytetrafluoroethylene (U.S. patent application Ser. No. 11/607,734), polyethylene (U.S. Pat. App. Pub. No. 2007-0141628), and polystyrene (U.S. Pat. App. Pub. No. 2007-0261775). Examples of various target surface-binding peptides are provided in Table A.

### Target Surface-Binding Peptides

As described herein, target surface-binding peptides are single chain peptides having strong affinity for a target surface. The target surface-binding peptide are from about 7 amino acids to about 60 amino acids in length, more preferably, from about 7 amino acids to about 25 amino acids in length, most preferably from about 7 to about 20 amino acids in length. In one embodiment, the target surface-binding peptide is selected from a peptide library based on affinity for the target surface (i.e. a biopanned peptide). In another embodiment, the target surface-binding peptide may be identified using phage display. In another embodiment, the target surface-binding peptide may be empirically generated (Rothe et al., supra).

The target surface-binding peptide may be strong affinity for a particulate benefit agent surface (e.g. a pigment, a sunscreen agent, a whitening agent, etc.), a polymeric coating applied to a particulate benefit agent (e.g. a coated pigment), a clay, calcium carbonate or a body surface. Examples of various target binding peptides are provided in Table A.

### Body Surface-Binding Peptides

The target surface-binding peptide may be a body surface-binding peptide. Peptides having an affinity for a body surface have been described in (U.S. Pat. Nos. 7,220,405 and 7,285,264; U.S. Pat. App. Pub. Nos. 2005/0226839, 2005/0249682, 2007/0065387, 2007/0067924, 2007/0196305, 2007/0110686, 2006/0073111, and 2006/0199206; U.S. patent application Ser. No. 11/877,692; U.S. patent application Ser. No. 11/939,583; and Int'l Pat. App. Pub. No. WO2004048399). Examples of various body surface-binding peptides are also provided in Table A.

TABLE A
Examples of Target Surface-Binding Peptides

| Target | | SEQ ID | |
|---|---|---|---|
| Surface | Amino Acid Sequence | NO: | Reference |
| Hair | RVPNKTVTVDGA | 35 | US 2005/0226839 |
| | | | U.S. Pat. No. 7,220,405 |
| Hair | DRHKSKYSSTKS | 36 | US 2005/0226839 |
| | | | U.S. Pat. No. 7,220,405 |
| Hair | KNFPQQKEFPLS | 37 | US 2005/0226839 |
| | | | U.S. Pat. No. 7,220,405 |
| Hair | QRNSPPAMSRRD | 38 | US 2005/0226839 |
| | | | U.S. Pat. No. 7,220,405 |
| Hair | TRKPNMPHGQYL | 39 | US 2005/0226839 |
| | | | U.S. Pat. No. 7,220,405 |
| Hair | KPPHLAKLPFTT | 40 | US 2005/0226839 |
| | | | U.S. Pat. No. 7,220,405 |
| Hair | NKRPPTSHRIHA | 41 | US 2005/0226839 |
| | | | U.S. Pat. No. 7,220,405 |
| Hair | NLPRYQPPCKPL | 42 | US 2005/0226839 |
| | | | U.S. Pat. No. 7,220,405 |
| Hair | RPPWKKPIPPSE | 43 | US 2005/0226839 |
| | | | U.S. Pat. No. 7,220,405 |
| Hair | RQRPKDHFFSRP | 44 | US 2005/0226839 |
| | | | U.S. Pat. No. 7,220,405 |
| Hair | SVPNKXVTVDGX | 45 | US 2005/0226839 |
| | | | U.S. Pat. No. 7,220,405 |
| Hair | TTKWRHRAPVSP | 46 | US 2005/0226839 |
| | | | U.S. Pat. No. 7,220,405 |
| Hair | WLGKNRIKPRAS | 47 | US 2005/0226839 |
| | | | U.S. Pat. No. 7,220,405 |
| Hair | SNFKTPLPLTQS | 48 | US 2005/0226839 |
| | | | U.S. Pat. No. 7,220,405 |
| Hair | SVSVGMKPSPRP | 49 | US 2005/0226839 |
| | | | U.S. Pat. No. 7,220,405 |
| Hair | DLHTVYH | 50 | US 2005/0226839 |
| | | | U.S. Pat. No. 7,220,405 |
| Hair | HIKPPTR | 51 | US 2005/0226839 |
| | | | U.S. Pat. No. 7,220,405 |
| Hair | HPVWPAI | 52 | US 2005/0226839 |
| | | | U.S. Pat. No. 7,220,405 |
| Hair | MPLYYLQ | 53 | US 2005/0226839 |
| | | | U.S. Pat. No. 7,220,405 |
| Hair | HLTVPWRGGGSAVPFYSHSQIT | 54 | US 2005/0226839 |
| | LPNH | | U.S. Pat. No. 7,220,405 |
| Hair | GPHDTSSGGVRPNLHHTSKKE | 55 | US 2005/0226839 |
| | KRENRKVPFYSHSVTSRGNV | | U.S. Pat. No. 7,220,405 |
| Hair | KHPTYRQ | 56 | US 2005/0226839 |
| | | | U.S. Pat. No. 7,220,405 |
| Hair | HPMSAPR | 57 | US 2005/0226839 |
| | | | U.S. Pat. No. 7,220,405 |
| Hair | MPKYYLQ | 58 | US 2005/0226839 |
| | | | U.S. Pat. No. 7,220,405 |
| Hair | MHAHSIA | 59 | US 2005/0226839 |
| | | | U.S. Pat. No. 7,220,405 |
| Hair | AKPISQHLQRGS | 60 | US 2005/0226839 |
| | | | U.S. Pat. No. 7,220,405 |
| Hair | APPTPAAASATT | 61 | US 2005/0226839 |
| | | | U.S. Pat. No. 7,220,405 |
| Hair | DPTEGARRTIMT | 62 | US 2005/0226839 |
| | | | U.S. Pat. No. 7,220,405 |
| Hair | LDTSFPPVPFHA | 63 | US 2005/0226839 |
| | | | U.S. Pat. No. 7,220,405 |
| Hair | LDTSFHQVPFHQ | 64 | US 2005/0226839 |
| | | | U.S. Pat. No. 7,220,405 |
| Hair | LPRIANTWSPS | 65 | US 2005/0226839 |
| | | | U.S. Pat. No. 7,220,405 |
| Hair | RTNAADHPAAVT | 66 | US 2005/0226839 |
| | | | U.S. Pat. No. 7,220,405 |
| | | | US 2007/0065387 |
| Hair | SLNWVTIPGPKI | 67 | US 2005/0226839 |
| | | | U.S. Pat. No. 7,220,405 |
| Hair | TDMQAPTKSYSN | 68 | US 2005/0226839 |
| | | | U.S. Pat. No. 7,220,405 |
| Hair | TIMTKSPSLSCG | 69 | US 2005/0226839 |
| | | | U.S. Pat. No. 7,220,405 |
| Hair | TPALDGLRQPLR | 70 | US 2005/0226839 |
| | | | U.S. Pat. No. 7,220,405 |
| Hair | TYPASRLPLLAP | 71 | US 2005/0226839 |
| | | | U.S. Pat. No. 7,220,405 |
| Hair | AKTHKHPAPSYS | 72 | US 2005/0226839 |
| | | | U.S. Pat. No. 7,220,405 |
| Hair | TDPTPFSISPER | 73 | US 2005/0226839 |
| | | | U.S. Pat. No. 7,220,405 |
| Hair | SQNWQDSTSYSN | 74 | US 2005/0226839 |
| | | | U.S. Pat. No. 7,220,405 |
| Hair | WHDKPQNSSKST | 75 | US 2005/0226839 |
| | | | U.S. Pat. No. 7,220,405 |
| Hair | LDVESYKGTSMP | 76 | US 2005/0226839 |
| | | | U.S. Pat. No. 7,220,405 |
| Hair | NTPKENW | 77 | WO2004/48399 |
| Hair | NTPASNR | 78 | WO2004/48399 |
| Hair | PRGMLST | 79 | WO2004/48399 |
| Hair | PPTYLST | 80 | WO2004/48399 |
| Hair | TIPTHRQHDYRS | 81 | WO2004/48399 |
| Hair | TPPTHRL | 82 | WO2004/048399 |
| Hair | LPTMSTP | 83 | WO2004/048399 |
| Hair | LGTNSTP | 84 | WO2004/048399 |
| Hair | TPLTGSTNLLSS | 85 | WO2004/048399 |
| Hair | TPLTKET | 86 | WO2004/048399 |
| Hair | KQSHNPP | 87 | WO2004/048399 |
| Hair | QQSHNPP | 88 | WO2004/048399 |
| Hair | TQPHNPP | 89 | WO2004/048399 |
| Hair | STNLLRTSTVHP | 90 | WO2004/048399 |
| Hair | HTQPSYSSTNLF | 91 | WO2004/048399 |
| Hair | SLLSSHA | 92 | WO2004/048399 |
| Hair | QQSSISLSSHAV | 93 | WO2004/048399 |
| Hair | NASPSSL | 94 | WO2004/048399 |
| Hair | HSPSSLR | 95 | WO2004/048399 |
| Hair | K H/R/N SHHTH | 96 | WO2004/048399 |
| Hair | E H/R/N SHHTH | 97 | WO2004/048399 |
| Hair | SHHTHYGQPGPV | 98 | WO2004/048399 |
| Hair | LESTSLL | 99 | WO2004/048399 |
| Hair | DLTLPFH | 100 | US 2007/065387 |
| Hair | RTNAADHP | 101 | US 2007/067924 |
| Hair | IPWWNIRAPLNA | 102 | US 2007/0067924 |
| Hair | EQISGSLVAAPWEGEGER | 103 | US 11/877,692 |
| Hair | TPPELLHGAPRS | 104 | US 11/877,692 |
| “IB5A” | | | |
| Hair | LDTSFHQVPFHQKRKRKD | 105 | US 11/877,692 |
| Hair | EQISGSLVAAPWKRKRKD | 106 | US 11/877,692 |
| Hair | TPPELLHGDPRSKRKRKD | 107 | US 11/877,692 |
| Hair | NTSQLSTEGEGED | 108 | US 11/877,692 |
| Hair | TPPELLHGDPRSC | 109 | US 2007/067924 |
| Hair | HINKTNPHQGNHHSEKTQRQ | 110 | US 11/939,583 |
| “MEA4” | | | |
| Hair | HAHKNQKETHQRHAA | 111 | US 11/939,583 |
| Hair | HEHKNQKETHQRHAA | 112 | US 11/939,583 |
| | | | U.S. Pat. No. 7,285,264 |
| Hair | HNHMQERYTEPQHSPSVNGL | 113 | US 11/939,583 |
| Hair | THSTHNHGSPRHTNADA | 114 | US 2007/196305 |
| Hair | GSCVDTHKADSCVANNGPAT | 115 | US 11/939,583 |
| “HP1” | | | |
| Hair | AQSQLPDKHSGLHERAPQRY | 116 | US 11/939,583 |
| “HP2” | | | |
| Hair | AQSQLPAKHSGLHERAPQRY | 117 | US 11/939,583 |
| Hair | AQSQLPEKHSGLHERAPQRY | 118 | US 11/939,583 |
| Hair | TDMMHNHSDNSPPHRRSPRN | 119 | US 11/939,583 |
| “HP3” | | | |
| Hair | TPPELAHTPHHLAQTRLTDR | 120 | US 11/939,583 |
| “HP4” | | | |
| Hair | RLLRLLRLLRLL | 121 | US 11/939,583 |
| Hair | TPPELLHGEPRS | 122 | US 11/939,583 |
| Hair | TPPELLHGAPRS | 123 | U.S. Pat. No. 7,285,264 |
| Hair (normal | EQISGSLVAAPW | 124 | US 2005/0226839 |
| and | | | U.S. Pat. No. 7,220,405 |
| bleached) | | | |
| Hair | NEVPARNAPWLV | 125 | US 2005/0226839 |
| (bleached) | | | U.S. Pat. No. 7,220,405 |
| Hair | NSPGYQADSVAIG | 126 | US 2005/0226839 |
| (bleached) | | | U.S. Pat. No. 7,220,405 |
| Hair | AKPISQHLQRGS | 127 | US 2005/0226839 |
| (bleached) | | | U.S. Pat. No. 7,220,405 |
| Hair | LDTSFPPVPFHA | 128 | US 2005/0226839 |
| (bleached) | | | U.S. Pat. No. 7,220,405 |
| Hair | SLNWVTIPGPKI | 129 | US 2005/0226839 |
| (bleached) | | | U.S. Pat. No. 7,220,405 |
| Hair | TQDSAQKSPSPL | 130 | US 2005/0226839 |
| (bleached) | | | U.S. Pat. No. 7,220,405 |
| Hair | KELQTRNVVQRE | 131 | US 2005/0226839 |
| (bleached) | | | U.S. Pat. No. 7,220,405 |
| Hair | QRNSPPAMSRRD | 132 | US 2005/0226839 |
| (bleached) | | | U.S. Pat. No. 7,220,405 |
| Hair | TPTANQFTQSVP | 133 | US 2005/0226839 |
| (bleached) | | | U.S. Pat. No. 7,220,405 |
| Hair | AAGLSQKHERNR | 134 | US 2005/0226839 |
| (bleached) | | | U.S. Pat. No. 7,220,405 |
| Hair | ETVHQTPLSDRP | 135 | US 2005/0226839 |
| (bleached) | | | U.S. Pat. No. 7,220,405 |
| Hair | KNFPQQKEFPLS | 136 | US 2005/0226839 |
| (bleached) | | | U.S. Pat. No. 7,220,405 |
| Hair | LPALHIQRHPRM | 137 | US 2005/0226839 |
| (bleached) | | | U.S. Pat. No. 7,220,405 |
| Hair | QPSHSQSHNLRS | 138 | US 2005/0226839 |
| (bleached) | | | U.S. Pat. No. 7,220,405 |
| Hair | RGSQKSKPPRPP | 139 | US 2005/0226839 |
| (bleached) | | | U.S. Pat. No. 7,220,405 |
| Hair | THTQKTPLLYYH | 140 | US 2005/0226839 |
| (bleached) | | | U.S. Pat. No. 7,220,405 |
| Hair | TKGSSQAILKST | 141 | US 2005/0226839 |
| (bleached) | | | U.S. Pat. No. 7,220,405 |
| Hair (normal | TAATTSP | 142 | US 2005/0226839 |
| and | | | U.S. Pat. No. 7,220,405 |
| bleached) | | | |
| Hair | LGIPQNL | 143 | US 2005/0226839 |
| (bleached) | | | U.S. Pat. No. 7,220,405 |
| Hair | THSTHNHGSPRHTNADAGNP | 144 | US 2007/0065387 |
| (Conditioner | | | US 2007/0196305 |
| resistant) | | | |
| Hair | QQHKVHHQNPDRSTQDAHHS | 145 | US 2007/0196305 |
| (Conditioner | | | |
| resistant) | | | |
| Hair | HHGTHHNATKQKNHV | 146 | US 2007/0196305 |
| (Conditioner | | | |
| resistant) | | | |
| Hair | STLHKYKSQDPTPHH | 147 | US 2007/0196305 |
| (Conditioner | | | |
| resistant) | | | |
| Hair | SVSVGMKPSPRP | 148 | US 2007/0196305 |
| (Conditioner | | | |
| resistant) | | | |
| Hair | TPPTNVLMLATK | 149 | US 2006/0073111 |
| (shampoo | | | |
| resistant) | | | |
| “HB1” | | | |
| Hair | TPPELLHGDPRS | 150 | US 2006/0073111 |
| (shampoo | | | |
| resistant) | | | |
| Hair | NTSQLST | 151 | US 2007/0067924 |
| (shampoo | | | U.S. Pat. No. 7,285,264 |
| resistant) | | | |
| “KF11” | | | |
| Hair | STLHKYKSQDPTPHH | 152 | US 2007/0196305 |
| (conditioner | | | |
| resistant) | | | |
| Hair | GMPAMHWIHPFA | 153 | US 2006/0073111 |
| (shampoo | | | U.S. Pat. No. 7,285,264 |
| and | | | |
| conditioner | | | |
| resistant) | | | |
| “Gray1” | | | |
| Hair | HDHKNQKETHQRHAA | 154 | US 2006/0073111 |
| (shampoo | | | U.S. Pat. No. 7,285,264 |
| and | | | |
| conditioner | | | |
| resistant) | | | |
| “Gray 3” | | | |
| Hair | HNHMQERYTDPQHSPSVNGL | 155 | US 2006/0073111 |
| (shampoo | | | U.S. Pat. No. 7,285,264 |
| and | | | |
| conditioner | | | |
| resistant) | | | |
| Hair | TAEIQSSKNPNPHPQRSWTN | 156 | US 2006/0073111 |
| (shampoo | | | U.S. Pat. No. 7,285,264 |
| and | | | |
| conditioner | | | |
| resistant) | | | |
| “Gray 5” | | | |
| Hair (dyed) | SSADFASFGFFGFSAASADSR | 157 | US 12/198,382 |
| Hair (dyed) | SSFAEAWSRAWPRAEVFFPSR | 158 | US 12/198,382 |
| | GY | | |
| Hair (dyed) | SSFSVNEPHAWMAPLSR | 159 | US 12/198,382 |
| Hair (dyed) | SSFSWVYGHGGLGFASR | 160 | US 12/198,382 |
| Hair (dyed) | SSFVSWSPYKSPPELSR | 161 | US 12/198,382 |
| Hair (dyed) | SSFYGSSAFVSSGVSVAYGSR | 162 | US 12/198,382 |
| Hair (dyed) | SSGSVAVSAEASWFSGVAASR | 163 | US 12/198,382 |
| Hair (dyed) | SSHDEHYQYHYYSSR | 164 | US 12/198,382 |
| Hair (dyed) | SSHYYYNDYDHQSSR | 165 | US 12/198,382 |
| Hair (dyed) | SSLFNMYGHQSVLGPSR | 166 | US 12/198,382 |
| Hair (dyed) | SSLFSDVHYGSNKALSR | 167 | US 12/198,382 |
| Hair (dyed) | SSLLSDFHYGDMWDASR | 168 | US 12/198,382 |
| Hair (dyed) | SSNYNYNYNYQYSSR | 169 | US 12/198,382 |
| Hair (dyed) | SSNYNYNYNYQYSSREGEGER | 170 | US 12/198,382 |
| Hair (dyed) | SSNYNYNYNYQYSSRKRKRKD | 171 | US 12/198,382 |
| Hair (dyed) | SSQYYQDYQYYHSSR | 172 | US 12/198,382 |
| Hair (dyed) | SSSCMGSHNPRMSVEESTRNC | 173 | US 12/198,382 |
| | SR | | |
| Hair (dyed) | SSSCNNNWFYSSTLPGGDHAC | 174 | US 12/198,382 |
| | SR | | |
| Hair (dyed) | SSSCYDVECSSFVAWMRGPS | 175 | US 12/198,382 |
| | SSR | | |
| Hair (dyed) | SSSFAASSAFSFLVDAVAWSR | 176 | US 12/198,382 |
| Hair (dyed) | SSSFAYLVPDDGWLSSR | 177 | US 12/198,382 |
| Hair (dyed) | SSSGAVFSSGGADAGWGVWS | 178 | US 12/198,382 |
| | R | | |
| Hair (dyed) | SSSSADAAYGHCCGAGFSTFS | 179 | US 12/198,382 |
| | SR | | |
| Hair (dyed) | SSSSDVHNSIIGWDFYHSRGSS | 180 | US 12/198,382 |
| | R | | |
| Hair (dyed) | SSSSLDFFSYSAFSGGVAESR | 181 | US 12/198,382 |
| Hair (dyed) | SSSSNDSNVSWFHYYASGLTS | 182 | US 12/198,382 |
| | SR | | |
| Hair (dyed) | SSVDYEVPLAVAAEWGFSVSR | 183 | US 12/198,382 |
| Hair (dyed) | SSYHYDYDHYYESSR | 184 | US 12/198,382 |
| Hair (dyed) | SSYYNYHYQYQDSSR | 185 | US 12/198,382 |
| Hair (dyed) | SSYYYDYYQQDYSSR | 186 | US 12/198,382 |
| Hair and skin | KRGRHKRPKRHK | 187 | US 2007/0065387 |
| (Empirical) | | | US 2007/0110686 |
| | | | US 2007/0067924 |
| Hair and skin | RLLRLLR | 188 | US 2007/0065387 |
| (Empirical) | | | US 2007/0110686 |
| Hair and skin | HKPRGGRKKALH | 189 | US 2007/0065387 |
| (Empirical) | | | US 2007/0110686 |
| Hair and skin | KPRPPHGKKHRPKHRPKK | 190 | US 2007/0065387 |
| (Empirical) | | | US 2007/0110686 |
| Hair and skin | RGRPKKGHGKRPGHRARK | 191 | US 2007/0065387 |
| (Empirical) | | | US 2007/0110686 |
| Skin | TPFHSPENAPGS | 192 | US 11/877,692 |
| | | | US 2005/0249682 |
| Skin | TPFHSPENAPGSK | 193 | US 2007/0110686 |
| Skin | TPFHSPENAPGSGGGS | 194 | US 2007/0110686 |
| Skin | TPFHSPENAPGSGGGSS | 195 | US 2007/0110686 |
| Skin | TPFHSPENAPGSGGG | 196 | US 2007/0110686 |
| Skin | FTQSLPR | 197 | US 11/877,692 |
| | | | US 2005/0249682 |
| Skin | KQATFPPNPTAY | 198 | US 11/877,692 |
| | | | US 2005/0249682 |
| | | | WO2004/048399 |
| Skin | HGHMVSTSQLSI | 199 | US 11/877,692 |
| | | | US 2005/0249682 |
| | | | WO2004/048399 |
| Skin | LSPSRMK | 200 | US 11/877,692 |
| | | | US 2005/0249682 |
| | | | WO2004/048399 |
| Skin | LPIPRMK | 201 | US 2005/0249682 |
| | | | WO2004/048399 |
| Skin | HQRPYLT | 202 | US 2005/0249682 |
| | | | WO2004/048399 |
| Skin | FPPLLRL | 203 | US 2005/0249682 |
| | | | WO2004/048399 |
| Skin | QATFMYN | 204 | WO2004/048399 |
| Skin | VLTSQLPNHSM | 205 | WO2004/048399 |
| Skin | HSTAYLT | 206 | WO2004/048399 |
| Skin | APQQRPMKTFNT | 207 | WO2004/048399 |
| Skin | APQQRPMKTVQY | 208 | WO2004/048399 |
| Skin | PPWLDLL | 209 | WO2004/048399 |
| Skin | PPWTFPL | 210 | WO2004/048399 |
| Skin | SVTHLTS | 211 | WO2004/048399 |
| Skin | VITRLTS | 212 | WO2004/048399 |
| Skin | DLKPPLLALSKV | 213 | WO2004/048399 |
| Skin | SHPSGALQEGTF | 214 | WO2004/048399 |
| Skin | FPLTSKPSGACT | 215 | WO2004/048399 |
| Skin | DLKPPLLALSKV | 216 | WO2004/048399 |
| Skin | PLLALHS | 217 | WO2004/048399 |
| Skin | VPISTQI | 218 | WO2004/048399 |
| Skin | YAKQHYPISTFK | 219 | WO2004/048399 |
| Skin | HSTAYLT | 220 | WO2004/048399 |
| Skin | STAYLVAMSAAP | 221 | WO2004/048399 |
| Skin (Body | SVSVGMKPSPRP | 222 | US 11/877,692 |
| Wash | | | US 2006/0199206 |
| Resistant) | | | |
| Skin (Body | TMGFTAPRFPHY | 223 | US 11/877,692 |
| Wash | | | US 2006/0199206 |
| Resistant) | | | |
| Skin (Body | NLQHSVGTSPVW | 224 | US 11/877,692 |
| Wash | | | US 2006/0199206 |
| Resistant) | | | |
| Skin (Body | QLSYHAYPQANHHAP | 225 | US 11/877,692 |
| Wash | | | US 2006/0199206 |
| Resistant) | | | |
| Skin (Body | NQAASITKRVPY | 226 | US 2006/0199206 |
| Wash | | | |
| Resistant) | | | |
| Skin (Body | SGCHLVYDNGFCDH | 227 | US 11/877,692 |
| Wash | | | US 2006/0199206 |
| Resistant) | | | |
| Skin (Body | ASCPSASHADPCAH | 228 | US 11/877,692 |
| Wash | | | US 2006/0199206 |
| Resistant) | | | |
| Skin (Body | NLCDSARDSPRCKV | 229 | US 11/877,692 |
| Wash | | | US 2006/0199206 |
| Resistant) | | | |
| Skin (Body | NHSNWKTAADFL | 230 | US 11/877,692 |
| Wash | | | US 2006/0199206 |
| Resistant) | | | |
| Skin (Body | GSSTVGRPLSYE | 231 | US 2006/0199206 |
| Wash | | | |
| Resistant) | | | |
| Skin (Body | SDTISRLHVSMT | 232 | US 11/877,692 |
| Wash | | | US 2006/0199206 |
| Resistant) | | | |
| Skin (Body | SPLTVPYERKLL | 233 | US 2006/0199206 |
| Wash | | | |
| Resistant) | | | |
| Skin (Body | SPYPSWSTPAGR | 234 | US 11/877,692 |
| Wash | | | US 2006/0199206 |
| Resistant) | | | |
| Skin (Body | VQPITNTRYEGG | 235 | US 2006/0199206 |
| Wash | | | |
| Resistant) | | | |
| Skin (Body | WPMHPEKGSRWS | 236 | US 2006/0199206 |
| Wash | | | |
| Resistant) | | | |
| Skin (Body | DACSGNGHPNNCDR | 237 | US 11/877,692 |
| Wash | | | US 2006/0199206 |
| Resistant) | | | |
| Skin | DHCLGRQLQPVCYP | 238 | US 2006/0199206 |
| (Body | | | |
| Wash | | | |
| Resistant) | | | |
| Skin | DWCDTIIPGRTCHG | 239 | US 11/877,692 |
| (Body | | | US 2006/0199206 |
| Wash | | | |
| Resistant) | | | |
| Fingernail | ALPRIANTWSPS | 240 | US 2005/0226839 |
| | | | U.S. Pat. No. 7,220,405 |
| Fingernail | YPSFSPTYRPAF | 241 | US 2005/0226839 |
| and Hair | | | U.S. Pat. No. 7,220,405 |
| Tooth | AHPESLGIKYALDGNSDPHA | 242 | US 11/877,692 |
| (pellicle) | | | |
| Tooth | ASVSNYPPIHHLATSNTTVN | 243 | US 11/877,692 |
| (pellicle) | | | |
| Tooth | DECMEPLNAAHCWR | 244 | US 11/877,692 |
| (pellicle) | | | |
| Tooth | DECMHGSDVEFCTS | 245 | US 11/877,692 |
| (pellicle) | | | |
| Tooth | DLCSMQMMNTGCHY | 246 | US 11/877,692 |
| (pellicle) | | | |
| Tooth | DLCSSPSTWGSCIR | 247 | US 11/877,692 |
| (pellicle) | | | |
| Tooth | DPNESNYENATTVSQPTRHL | 248 | US 11/877,692 |
| (pellicle) | | | |
| Tooth | EPTHPTMRAQMHQSLRSSSP | 249 | US 11/877,692 |
| (pellicle) | | | |
| Tooth | GNTDTTPPNAVMEPTVQHKW | 250 | US 11/877,692 |
| (pellicle) | | | |
| Tooth | NGPDMVQSVGKHKNS | 251 | US 11/877,692 |
| (pellicle) | | | |
| Tooth | NGPEVRQIPANFEKL | 252 | US 11/877,692 |
| (pellicle) | | | |
| Tooth | NNTSADNPPETDSKHHLSMS | 253 | US 11/877,692 |
| (pellicle) | | | |
| Tooth | NNTWPEGAGHTMPSTNIRQA | 254 | US 11/877,692 |
| (pellicle) | | | |
| Tooth | NPTATPHMKDPMHSNAHSSA | 255 | US 11/877,692 |
| (pellicle) | | | |
| Tooth | NPTDHIPANSTNSRVSKGNT | 256 | US 11/877,692 |
| (pellicle) | | | |
| Tooth | NPTDSTHMMHARNHE | 257 | US 11/877,692 |
| (pellicle) | | | |
| Tooth | QHCITERLHPPCTK | 258 | US 11/877,692 |
| (pellicle) | | | |
| Tooth | TPCAPASFNPHCSR | 259 | US 11/877,692 |
| (pellicle) | | | |
| Tooth | TPCATYPHFSGCRA | 260 | US 11/877,692 |
| (pellicle) | | | |
| Tooth | WCTDFCTRSTPTSTSRSTTS | 261 | US 11/877,692 |
| (pellicle) | | | |
| Tooth | APPLKTYMQERELTMSQNKD | 262 | US 11/877,692 |
| (enamel) | | | |
| Tooth | EPPTRTRVNNHTVTVQAQQH | 263 | US 11/877,692 |
| (enamel) | | | |
| Tooth | GYCLRGDEPAVCSG | 264 | US 11/877,692 |
| (enamel) | | | |
| Tooth | LSSKDFGVTNTDQRTYDYTT | 265 | US 11/877,692 |
| (enamel) | | | |
| Tooth | NFCETQLDLSVCTV | 266 | US 11/877,692 |
| (enamel) | | | |
| Tooth | NTCQPTKNATPCSA | 267 | US 11/877,692 |
| (enamel) | | | |
| Tooth | PSEPERRDRNIAANAGRFNT | 268 | US 11/877,692 |
| (enamel) | | | |
| Tooth | THNMSHFPPSGHPKRTAT | 269 | US 11/877,692 |
| (enamel) | | | |
| Tooth | TTCPTMGTYHVCWL | 270 | US 11/877,692 |
| (enamel) | | | |
| Tooth | YCADHTPDPANPNKICGYSH | 271 | US 11/877,692 |
| (enamel) | | | |
| Tooth | AANPHTEWDRDAFQLAMPPK | 272 | US 11/877,692 |
| (enamel) | | | |
| Tooth | DLHPMDPSNKRPDNPSDLHT | 273 | US 11/877,692 |
| (enamel) | | | |
| Tooth | ESCVSNALMNQCIY | 274 | US 11/877,692 |
| (enamel) | | | |
| Tooth | HNKADSWDPDLPPHAGMSLG | 275 | US 11/877,692 |
| (enamel) | | | |
| Tooth | LNDQRKPGPPTMPTHSPAVG | 276 | US 11/877,692 |
| (enamel) | | | |
| Tooth | NTCATSPNSYTCSN | 277 | US 11/877,692 |
| (enamel) | | | |
| Tooth | SDCTAGLVPPLCAT | 278 | US 11/877,692 |
| (enamel) | | | |
| Tooth | TIESSQHSRTHQQNYGSTKT | 279 | US 11/877,692 |
| (enamel) | | | |
| Tooth | VGTMKQHPTTTQPPRVSATN | 280 | US 11/877,692 |
| (enamel) | | | |
| Tooth | YSETPNDQKPNPHYKVSGTK | 281 | US 11/877,692 |
| (enamel) | | | |
| PMMA | IPWWNIRAPLNA | 282 | US 2007/0265431 |
| PMMA | TAVMNVVNNQLS | 283 | US 2007/0265431 |
| PMMA | VPWWAPSKLSMQ | 284 | US 2007/0265431 |
| PMMA | MVMAPHTPRARS | 285 | US 2007/0265431 |
| PMMA | TYPNWAHLLSHY | 286 | US 2007/0265431 |
| PMMA | TPWWRIT | 287 | US 2007/0265431 |
| PMMA | DLTLPFH | 288 | US 2007/0265431 |
| PMMA | GTSIPAM | 289 | US 2007/0265431 |
| PMMA | HHKHVVA | 290 | US 2007/0265431 |
| PMMA | HHHKHFM | 291 | US 2007/0265431 |
| PMMA | HHHRHQG | 292 | US 2007/0265431 |
| PMMA | HHWHAPR | 293 | US 2007/0265431 |
| PMMA | APWHLSSQYSGT | 294 | US 2007/0065387 |
| PMMA | GYCLRVDEPTVCSG | 295 | US 2007/0065387 |
| PMMA | HIHPSDNFPHKNRTH | 296 | US 2007/0065387 |
| PMMA | HTHHDTHKPWPTDDHRNSSV | 297 | US 2007/0065387 |
| PMMA | PEDRPSRTNALHHNAHHHNA | 298 | US 2007/0065387 |
| PMMA | TPHNHATTNHHAGKK | 299 | US 2007/0065387 |
| PMMA | EMVKDSNQRNTRISS | 300 | US 2007/0065387 |
| PMMA | HYSRYNPGPHPL | 301 | US 2007/0065387 |
| PMMA | IDTFYMSTMSHS | 302 | US 2007/0065387 |
| PMMA | PMKEATHPVPPHKHSETPTA | 303 | US 2007/0065387 |
| PMMA | YQTSSPAKQSVG | 304 | US 2007/0065387 |
| PMMA | HLPSYQITQTHAQYR | 305 | US 2007/0065387 |
| PMMA | TTPKTTYHQSRAPVTAMSEV | 306 | US 2007/0065387 |
| PMMA | DRIHHKSHHVTTNHF | 307 | US 2007/0065387 |
| PMMA | WAPEKDYMQLMK | 308 | US 2007/0065387 |
| PP | TSDIKSRSPHHR | 309 | US 2007/0264720 |
| PP | HTQNMRMYEPWF | 310 | US 2007/0264720 |
| PP | LPPGSLA | 311 | US 2007/0264720 |
| PP | MPAVMSSAQVPR | 312 | US 2007/0264720 |
| PP | NQSFLPLDFPFR | 313 | US 2007/0264720 |
| PP | SILSTMSPHGAT | 314 | US 2007/0264720 |
| PP | SMKYSHSTAPAL | 315 | US 2007/0264720 |
| PTFE | ESSYSWSPARLS | 316 | US 11/607,734 |
| PTFE | GPLKLLHAWWQP | 317 | US 11/607,734 |
| PTFE | NALTRPV | 318 | US 11/607,734 |
| PTFE | SAPSSKN | 319 | US 11/607,734 |
| PTFE | SVSVGMKPSPRP | 320 | US 11/607,734 |
| PTFE | SYYSLPPIFHIP | 321 | US 11/607,734 |
| PTFE | TFTPYSITHALL | 322 | US 11/607,734 |
| PTFE | TMGFTAPRFPHY | 323 | US 11/607,734 |
| PTFE | TNPFPPPPSSPA | 324 | US 11/607,734 |
| PE | HNKSSPLTAALP | 325 | US 2007/0141628 |
| PE | LPPWKHKTSGVA | 326 | US 2007/0141628 |
| PE | LPWWLRDSYLLP | 327 | US 2007/0141628 |
| PE | VPWWKHPPLPVP | 328 | US 2007/0141628 |
| PE | HHKQWHNHPHHA | 329 | US 2007/0141628 |
| PE | HIFSSWHQMWHR | 330 | US 2007/0141628 |
| PE | WPAWKTHPILRM | 331 | US 2007/0141628 |
| Nylon | KTPPTRP | 332 | US 2007/0141629 |
| Nylon | VINPNLD | 333 | US 2007/0141629 |
| Nylon | KVWIVST | 334 | US 2007/0141629 |
| Nylon | AEPVAML | 335 | US 2007/0141629 |
| Nylon | AELVAML | 336 | US 2007/0141629 |
| Nylon | HSLRLDW | 337 | US 2007/0141629 |
| PS | TSTASPTMQSKIR | 338 | US 2007/0261775 |
| PS | KRNHWQRMHLSA | 339 | US 2007/0261775 |
| PS | SHATPPQGLGPQ | 340 | US 2007/0261775 |
| CA | ATTPPSGKAAAHSAARQKGN | 341 | US 61/016,708 |
| CA | DTIHPNKMKSPSSPL | 342 | US 61/016,708 |
| CA | NGNNHTDIPNRSSYTGGSFA | 343 | US 61/016,708 |
| CA | SDETGPQIPHRRPTW | 344 | US 61/016,708 |
| “(CA4)” | | | |
| Carbon black | MPPPLMQ | 345 | US 2005/0054752 |
| Carbon black | FHENWPS | 346 | US 2005/0054752 |
| Carbon black | RTAPTTPLLLSL | 347 | US 2005/0054752 |
| Carbon black | WHLSWSPVPLPT | 348 | US 2005/0054752 |
| Cromophtal | PHARLVG | 349 | US 2005/0054752 |
| yellow | | | |
| Cromophtal | NIPYHHP | 350 | US 2005/0054752 |
| yellow | | | |
| Cromophtal | TTMPAIP | 351 | US 2005/0054752 |
| yellow | | | |
| Cromophtal | HNLPPRS | 352 | US 2005/0054752 |
| yellow | | | |
| Cromophtal | AHKTQMGVRQPA | 353 | US 2005/0054752 |
| yellow | | | |
| Cromophtal | ADNVQMGVSHTP | 354 | US 2005/0054752 |
| yellow | | | |
| Cromophtal | AHNAQMGVSHPP | 355 | US 2005/0054752 |
| yellow | | | |
| Cromophtal | ADYVGMGVSHRP | 356 | US 2005/0054752 |
| yellow | | | |
| Cromophtal | SVSVGMKPSPRP | 357 | US 2005/0054752 |
| yellow | | | |
| Sunfast | YPNTALV | 358 | US 2005/0054752 |
| Magenta | | | |
| Sunfast | VATRIVS | 359 | US 2005/0054752 |
| Magenta | | | |
| Sunfast | HSLKNSMLTVMA | 360 | US 2005/0054752 |
| Magenta | | | |
| Sunfast Blue | NYPTQAP | 361 | US 2005/0054752 |
| Sunfast Blue | KCCYSVG | 362 | US 2005/0054752 |
| Sunfast Blue | RHDLNTWLPPVK | 363 | US 2005/0054752 |
| Sunfast Blue | EISLPAKLPSAS | 364 | US 2005/0054752 |
| Sunfast Blue | SVSVGMKPSPRP | 357 | US 2005/0054752 |
| Sunfast Blue | SDYVGMRPSPRH | 365 | US 2005/0054752 |
| Sunfast Blue | SDYVGMRLSPSQ | 366 | US 2005/0054752 |
| Sunfast Blue | SVSVGIQPSPRP | 367 | US 2005/0054752 |
| Sunfast Blue | YVSVGIKPSPRP | 368 | US 2005/0054752 |
| Sunfast Blue | YVCEGIHPCPRP | 369 | US 2005/0054752 |
| Iron Oxide | WAPEKDHMQLMK | 370 | Co-pending |
| | | | application |
| Iron Oxide | WAPEKDYMQLMK | 371 | Co-pending |
| | | | application |
| Iron Oxide | CPLDTPTHKTKHEYKTRCRH | 372 | Co-pending |
| | | | application |
| Iron Oxide | DHDHPRLHKRQEKSEHLH | 373 | Co-pending |
| | | | application |
| Iron Oxide | DSHHNHHKQDSRPQHRKTPN | 374 | Co-pending |
| | | | application |
| Iron Oxide | EGGNAPHHKPHHRKH | 375 | Co-pending |
| | | | application |
| Iron Oxide | HDSHRPLTQHGHRHSHVP | 376 | Co-pending |
| | | | application |
| Iron Oxide | HDSNHCSHSTRRPNCART | 377 | Co-pending |
| | | | application |
| Iron Oxide | ATRVDNTPASNPPSL | 378 | Co-pending |
| | | | application |
| Iron Oxide | DGIKPFHLMTPTLAN | 379 | Co-pending |
| | | | application |
| Iron Oxide | DITPPGSTHHRKPHRHQH | 380 | Co-pending |
| | | | application |
| Iron Oxide | DNLWPQPLNVEDDRY | 381 | Co-pending |
| | | | application |
| Iron Oxide | ENEKHRHNTHEALHSHFK | 382 | Co-pending |
| | | | application |
| Iron Oxide | GAIWPASSALMTEHNPTDNH | 383 | Co-pending |
| | | | application |
| Iron Oxide | GDTNQDTVMWYYTVN | 384 | Co-pending |
| | | | application |
| Iron Oxide | HNGPYGMLSTGKIHF | 385 | Co-pending |
| | | | application |
| Iron Oxide | LDGGYRDTPDNYLKG | 386 | Co-pending |
| | | | application |
| Iron Oxide | LHTKTENSHTNMKTT | 387 | Co-pending |
| | | | application |
| Iron Oxide | NAQYDPPTLNKGAVRKAAST | 388 | Co-pending |
| | | | application |
| Iron Oxide | NGNNHTDIPNRSSYT | 389 | Co-pending |
| | | | application |
| Iron Oxide | QSTNHHHPHAKHPRVNTH | 390 | Co-pending |
| | | | application |
| Iron Oxide | SNNDYVGTYPATAIQ | 391 | Co-pending |
| | | | application |
| Iron Oxide | STQHNLHDRNIYFVS | 392 | Co-pending |
| | | | application |
| Iron Oxide | TANNKTPAGAPNAAVGLAQR | 393 | Co-pending |
| | | | application |
| Iron Oxide | TEPTRISNYRSIPND | 394 | Co-pending |
| | | | application |
| Iron Oxide | THNPREHARHHHHNEYKH | 395 | Co-pending |
| | | | application |
| Iron Oxide | THPPCWYETNCIVQE | 396 | Co-pending |
| | | | application |
| Iron Oxide | TTNPHKPASHHHDHRPALRH | 397 | Co-pending |
| | | | application |
| Iron Oxide | WLVADNATDGHSHQK | 398 | Co-pending |
| | | | application |
| Iron Oxide | YTDSMSDQTPEFAKY | 399 | Co-pending |
| | | | application |
| Cotton Fabric | SILPYPY | 400 | US 2005/0054752 |
| Cotton Fabric | STASYTR | 401 | US 2005/0054752 |
| Polyester/cotton | LPVRPWT | 402 | US 2005/0054752 |
| blend | | | |
| Polyester/cotton | SILPYPY | 400 | US 2005/0054752 |
| blend | | | |
| Hammermill | GNTPSRA | 403 | US 2005/0054752 |
| paper | | | |
| Hammermill | HAIYPRH | 404 | US 2005/0054752 |
| paper | | | |
| Hammermill | YQDSAKT | 405 | US 2005/0054752 |
| paper | | | |
| Hammermill | SILPYPY | 400 | US 2005/0054752 |
| paper | | | |
| Cellulose | VPRVTSI | 406 | US 2005/0054752 |
| Cellulose | MANHNLS | 407 | US 2005/0054752 |
| Cellulose | FHENWPS | 408 | US 2005/0054752 |
| Cellulose | THKTSTQRLLAA | 409 | US 2005/0054752 |
| Cellulose | KCCYVNVGSVFS | 410 | US 2005/0054752 |
| Cellulose | AHMQFRTSLTPH | 411 | US 2005/0054752 |
| Clay | GHGSPSNSHHGSKKCDMGNS | 412 | US 2007/0249805 |
| | RAKCKRL | | |
| Clay | SDRHNLRNSWSISRHCRRKQG | 413 | US 2007/0249805 |
| | RCLPAH | | |
| Clay | KKSNKGHHPSSKGKGPPWSE | 414 | US 2007/0249805 |
| | WDKKNGP | | |
| Clay | KKSNKGPHPSSKGKGPPWSE | 415 | US 2007/0249805 |
| | WDKKNGP | | |
| Clay | VGRHHSKAKQKRPHGGKGQN | 416 | US 2007/0249805 |
| | KN | | |
| Clay | VGRHHPKAKQKRPHGGKGQN | 417 | US 2007/0249805 |
| | KN | | |
| Clay | GRRPRARGRSRRGSTKT | 418 | US 2007/0249805 |
| Clay | LGVIRNHVVRGRRHHQHVR | 419 | US 2007/0249805 |
| Clay | QPGRPTEVHPELVRKSAYLVNP | 420 | US 2007/0249805 |
| | SEDIR | | |
| Clay | HRSEKPKNVKYKRGYWERGNQ | 421 | US 2007/0249805 |
| | KKHGPG | | |
| Clay | GSHKRRGSYALLRTRGVGRQA | 422 | US 2007/0249805 |
| | ELEHLL | | |
| Clay | VGEKPRRKSKGAKAKKARTKE | 423 | US 2007/0249805 |
| | EKLPKN | | |
| Clay | NKGHKQSGSPRHSNKKEKKTQ | 424 | US 2007/0249805 |
| | QKRGQP | | |
| Clay | HWGSQHKTGLRNHKRSRRDSL | 425 | US 2007/0249805 |
| | GKRGTD | | |
| Clay | KGWGSSSGPPGLTGKALGKGR | 426 | US 2007/0249805 |
| | LKPKKK | | |
| Calcium | RNNKGSKKVDDKRRKTVHNTK | 427 | US 11/828,539 |
| carbonate | SRAKYS | | |
| Calcium | RNNKGSKKVDDKRRKTVHNTK | 428 | US 11/828,539 |
| carbonate | SRAKHS | | |
| Calcium | RDNKGSKKVDDKRRKTVHNTK | 429 | US 11/828,539 |
| carbonate | SRAKYS | | |
| Calcium | RNNKGSKKVDDKRRKTVHSTK | 430 | US 11/828,539 |
| carbonate | SRAKYS | | |
| Calcium | RNNKGSRKVDDKRRKTVHNTK | 431 | US 11/828,539 |
| carbonate | SRAKYS | | |
| Calcium | RNNKGSKKADDKRRKTVHSTK | 432 | US 11/828,539 |
| carbonate | SRAKYS | | |
| Calcium | RNNKGSKKVDDKRRKAVHNKK | 433 | US 11/828,539 |
| carbonate | SRAKYS | | |
| Calcium | RNNKGSKKVDDKRRKTVHNTR | 434 | US 11/828,539 |
| carbonate | SRAKYS | | |
| Calcium | RNNKGSKKVDDKRRKTVHNTK | 435 | US 11/828,539 |
| carbonate | SRAKFS | | |
| Calcium | QRRKLRHPKEKWFGWSEKKVI | 436 | US 11/828,539 |
| carbonate | KKWSRK | | |
| Calcium | QRRKFRHPKEKWFGWSEKKVI | 437 | US 11/828,539 |
| carbonate | KXNGRP | | |
| Calcium | HKRLVQNKPHRTRKIEGWIKHM | 438 | US 11/828,539 |
| carbonate | VKRQH | | |
| Calcium | TRGHIMRPCWIGAMKQGVKKK | 439 | US 11/828,539 |
| carbonate | RTPGWR | | |
| Calcium | WKVKRRMVTRTYEFMGKKPCM | 440 | US 11/828,539 |
| carbonate | MLTKRL | | |
| Calcium | KKSNKGHHSKAKQKRPHGGKA | 441 | US 11/828,539 |
| carbonate | QNKNT | | |
| Calcium | RAHKERFVVRQIGRSQGYKTW | 442 | US 11/828,539 |
| carbonate | QCVRVA | | |
| Calcium | SQKPKGHKVKVVVKLCKRPYW | 443 | US 11/828,539 |
| carbonate | RMLNTA | | |
| Calcium | NHGCPVNWKVXNPPRGWQRL | 444 | US 11/828,539 |
| carbonate | NHCKWWN | | |
| Calcium | RNSRHKEWRRYKRTHVHSHEF | 445 | US 11/828,539 |
| carbonate | YHVECW | | |
| Calcium | HRSEKPKNVNYKRGYWERGN | 446 | US 11/828,539 |
| carbonate | QKKHGPG | | |
| Calcium | HERTRRGKPDRQKTTHEKRRQ | 447 | US 11/828,539 |
| carbonate | GLWIFM | | |
| Calcium | PWGTNKRQKHKVHEAKALKKS | 448 | US 11/828,539 |
| carbonate | LWYSNS | | |
| Calcium | RRGVVLCHTHRNKRIRLAYSVT | 449 | US 11/828,539 |
| carbonate | KKAWA | | |
| Calcium | ERIRWRRLSAEIRAHKWSVLKF | 450 | US 11/828,539 |
| carbonate | RLSCM | | |
| Calcium | KTKEKKKEVKLHKKSLSLVLLAD | 451 | US 11/828,539 |
| carbonate | LWRL | | |
| Calcium | LGKKHKQHSKVGHGKLSTRFLR | 452 | US 11/828,539 |
| carbonate | RSKLF | | |

*PMMA means polymethylmethacrylate, PP means polypropylene, PTFE means polytetrafluoroethylene, PE means polyethylene, PS means polystyrene, CA means cellulose acetate.

The body surface-binding peptide may be selected from the group consisting of hair-binding peptides, skin-binding peptides, nail-binding peptides, and teeth-binding peptides. The body surface-binding peptide may be selected from the group consisting of hair-binding peptides (SEQ ID NOs: 35-191), skin-binding peptides (SEQ ID NOs. 157-239), nail-binding peptides (SEQ ID NOs. 240-241), and teeth-binding peptides (SEQ ID NOs. 242-281).

### Production of Fusion Peptides Comprising an Inclusion Body Tag

The peptide of interest may be a small peptide that is appreciably soluble in the host cell and/or subject to endogenous proteolytic degradation. As such, the peptide of interest may be produced in an insoluble form (i.e. as inclusion bodies) by fusing the peptide of interest to an inclusion body tag (see U.S. patent application Ser. Nos. 11/782,836, 11/641,273, 11/641,936, 12/172,395, 11/641,981, and U.S. Pat. No. 7,427,656; each herein incorporated by reference).

The desired gene product may be a small bioactive peptide of interest that is appreciably soluble in the host cell and/or host cell liquid lysate under normal physiological conditions. Fusion of the peptide of interest to at least one inclusion body forming tags creates a fusion peptide that is insoluble in the host cell and/or host cell lysate under normal physiological conditions. Production of the peptide of interest is typically increased when expressed and accumulated in the form of an insoluble inclusion body as the peptide is generally more protected from proteolytic degradation. Furthermore, the insoluble fusion protein can be easily separated from the host cell lysate using centrifugation or filtration.

Typically, the fusion peptide is insoluble in an aqueous matrix at a temperature of 10° C. to 50° C., preferably 10° C. to 40° C. The aqueous matrix typically comprises a pH range of 5 to 12, preferably 6 to 10, and most preferably 6 to 8. The temperature, pH, and/or ionic strength of the aqueous matrix can be adjusted to obtain the desired solubility characteristics of the fusion peptide/inclusion body.

The peptide of interest may be expressed as a fusion peptide having the following general structure:

IBT-CL-POI

or

POI-CL-IBT

wherein;

IBT means at least one inclusion body tag;

CL means at least one cleavable peptide linker; and

POI means at least one peptide of interest.

As shown in the present examples, increasing the expression of several endogenous genes in E. coli increased the expression of a model fusion peptide comprising an inclusion body tag (IBT139) linked to a peptide of interest (HC776124) via an acid labile aspartic acid—proline dipeptide (see U.S. patent application Ser. No. 11/782,836).

### Cleavable Peptide Linkers

The use of cleavable peptide linkers is well known in the art. Fusion peptides comprising at least one inclusion body tag will typically include at least one cleavable sequence separating the inclusion body tag from the peptide of interest. The cleavable sequence facilitates separation of the inclusion body tag(s) from the peptide(s) of interest. The cleavable sequence may be provided by a portion of the inclusion body tag and/or the peptide of interest (e.g., inclusion of an acid cleavable aspartic acid—proline moiety). The cleavable sequence preferably includes in the fusion peptide at least one cleavable peptide linker between the inclusion body tag and the peptide of interest.

Means to cleave the peptide linkers are well known in the art and may include chemical hydrolysis, enzymatic cleavage agents, and combinations thereof. One or more chemically cleavable peptide linkers are included in the fusion construct to facilitate recovery of the peptide of interest from the inclusion body fusion protein.

Examples of chemical cleavage reagents include cyanogen bromide, which cleaves methionine residues; N-chloro succinimide, iodobenzoic acid or BNPS-skatole [2-(2-nitrophenylsulfenyl)-3-methylindole], which cleaves tryptophan residues; dilute acids, which cleave at aspartyl-prolyl bonds, and hydroxylamine, which cleaves at asparagine-glycine bonds at pH 9.0. See Gavit, P. and Better, M., J. Biotechnol., 79:127-136 (2000); Szoka et al., DNA, 5(1):11-20 (1986); and Walker, J. M., The Proteomics Protocols Handbook, 2005, Humana Press, Totowa, N.J. One or more aspartic acid—proline acid cleavable recognition sites (i.e., a cleavable peptide linker comprising one or more D-P dipeptide moieties) may preferably be included in the fusion protein construct to facilitate separation of the inclusion body tag(s) form the peptide of interest. The fusion peptide may include multiple regions encoding peptides of interest separated by one or more cleavable peptide linkers.

Moreover, one or more enzymatic cleavage sequences may be included in the fusion protein to facilitate recovery of the peptide of interest. Proteolytic enzymes and their respective cleavage site specificities are well known in the art. Preferably, the proteolytic enzyme is selected to cleave only the peptide linker separating the inclusion body tag and the peptide of interest. Examples of enzymes useful for cleaving the peptide linker include, but are not limited to Arg-C proteinase, Asp-N endopeptidase, chymotrypsin, clostripain, enterokinase, Factor Xa, glutamyl endopeptidase, Granzyme B, Achromobacter proteinase I, pepsin, proline endopeptidase, proteinase K, Staphylococcal peptidase I, thermolysin, thrombin, trypsin, and members of the Caspase family of proteolytic enzymes (e.g. Caspases 1-10) (Walker, J. M., supra). An example of a cleavage site sequence is provided by SEQ ID NO: 34 (Caspase-3 cleavage site; Thornberry et al. J. Biol. Chem., 272:17907-17911 (1997) and Tyas et al., EMBO Reports, 1(3):266-270 (2000)).

Typically, the cleavage step occurs after the insoluble inclusion bodies and/or insoluble fusion peptides are isolated from the cell lysate. The cells can be lysed using any number of means well known in the art, such as mechanical and/or chemical lysis. Methods to isolate the insoluble inclusion bodies/fusion peptides from the cell lysate are well known in the art, such as centrifugation, filtration, and combinations of these. Once recovered from the cell lysate, the insoluble inclusion bodies and/or fusion peptides can be treated with a chemical or enzymatic cleavage agent to cleave the inclusion body tag from the peptide of interest. The fusion protein and/or inclusion body may be diluted and/or dissolved in a suitable solvent prior to treatment with the cleavage agent. Alternatively, the cleavage step may be omitted if the inclusion body tag does not interfere with the activity of the peptide of interest.

After the cleavage step, preferably, the peptide of interest can be separated and/or isolated from the fusion protein and the inclusion body tags based on a differential solubility of the components. Parameters such as pH, salt concentration, and temperature may be adjusted to facilitate separation of the inclusion body tag from the peptide of interest. The peptide of interest may be soluble while the inclusion body tag and/or fusion protein is insoluble in the defined process matrix, typically aqueous. Alternatively, the peptide of interest may be insoluble while the inclusion body tag is soluble in the defined process matrix.

Optionally, the peptide of interest may be further purified using any number of well known purification techniques in the art such as ion exchange, gel purification techniques, and column chromatography (see U.S. Pat. No. 5,648,244).

### Peptide-Based Reagent for the Delivery of a Benefit Agent to a Body Surface

The present method can be use to produce peptide-based reagents comprising a first portion having affinity for a body surface and a second portion capable of being coupled to a benefit agent. The peptide-based reagent may be comprised of a first binding domain (binding “hand”) having multiple body surface-binding peptides (“fingers”) and a second binding domain (“hand”) having affinity for the benefit agent, wherein the second binding domain may be comprised of multiple benefit agent-binding peptides wherein the benefit agent is preferably a particulate benefit agent. The benefit agent is a molecule that imparts a desired functionality to a target material, such as hair, skin, etc., (see U.S. patent application Ser. Nos. 10/935,642, 11/074,473, and 11/696,380 for a list of typical benefit agents such as conditioners, pigments/colorants, fragrances, etc.). The benefit agent may be a peptide of interest itself or may be one or more molecules bound to, covalently or non-covalently, or associated with, the peptide of interest wherein the binding affinity of the peptide of interest is used to selectively target the benefit agent to the targeted material. The benefit agent may be a particulate benefit agent, such as a pigment or coated pigment.

The peptide of interest may comprise at least one region having an affinity for a targeted material and a plurality of regions having an affinity for a variety of benefit agents wherein the benefit agents may be the same or different. Examples of benefits agents include, but are not limited to, conditioners for personal care products, pigments, dye, fragrances, pharmaceutical agents (e.g., targeted delivery of cancer treatment agents), diagnostic/labeling agents, ultraviolet light blocking agents (i.e., active agents in sunscreen protectants), and antimicrobial agents (e.g., antimicrobial peptides). The single chain peptide-based reagent may range in length from about 14 to about 600 amino acids.

### Transformation and Expression

Preferred host cells are microbial hosts within the fungal or bacterial families and which grow over a wide range of temperatures, pH values, and solvent tolerances. It is contemplated that any microbial expression host can be used in the present process as many of the overexpressed genes are ubiquitous and/or expected to have structurally similar homologs in other species, i.e. expression of homologs of AroB, AroK, ProB, and Crl. Means to identify structurally similar biological molecules is well known in the art and means to identify structurally similar biological molecules is described above.

Transcription, translation, and the protein biosynthetic apparatus are universal genetic processes. Because of this, large-scale microbial growth and functional gene expression may utilize a wide range of simple or complex carbohydrates, organic acids and alcohols, i.e. methanol, saturated hydrocarbons such as methane or carbon dioxide in the case of photosynthetic or chemoautotrophic hosts. However, the functional genes may be regulated, repressed or depressed by specific growth conditions, which may include the form and amount of nitrogen, phosphorous, sulfur, oxygen, carbon or any trace micronutrient including small inorganic ions. In addition, the regulation of functional genes may be achieved by the presence or absence of specific regulatory molecules added to the culture and not typically considered nutrient or energy sources. Growth rate may also be an important regulatory factor in gene expression. Examples of host strains may include, but are not limited to bacterial species such as Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. The preferred host cells may be bacterial host cells, such as an Enterobacteriaceae or selected from the genera consisting of Escherichia, Salmonella, and Bacillus. Preferably, the host strain is Escherichia coli. The Escherichia coli host strain is preferably derived from a K-12 strain, more preferably E. coli K-12 substrain MG1655 (ATCC® 47076™).

### Fermentation Media

Fermentation media must contain suitable carbon substrates. Suitable substrates may include, but are not limited to, monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. L-arabinose is used to induce the present arabinose inducible expression system. As such, L-arabinose is typically not included in the fermentation media until expression of the desired chimeric gene (encoding the peptide or protein of interest) is desired. L-arabinose can be added at any time during the fermentation, although it is often preferable to induce expression only after a desired cell density/mass is achieved in the fermentor. It is contemplated that the source of carbon utilized in the present invention may encompass a wide variety of carbon containing substrates and will only be limited by the choice of organism. Preferred carbon substrates include glucose, fructose, and sucrose.

In addition to a carbon source, fermentation media may or must contain other components suitable and/or necessary for the growth of the cultures and promotion of the expression of the present fusion peptides. These are known to those skilled in the art and include minerals, salts, cofactors, buffers, etc.

### Culture Conditions

Suitable culture conditions can vary and depend on the chosen production host and are generally known in the art. Typically, cells are grown at a temperature in the range of about 25° C. to about 40° C. in an appropriate medium. Suitable growth media in the present invention are common commercially prepared media such as Luria Bertani (LB) broth, Sabouraud Dextrose (SD) broth or Yeast medium (YM) broth. Other defined or synthetic growth media may also be used and the appropriate medium for growth of the particular microorganism will be known by one skilled in the art of microbiology or fermentation science. The use of agents known to modulate catabolite repression directly or indirectly, e.g., cyclic adenosine 2′:3′-monophosphate, may also be incorporated into the fermentation medium.

Suitable pH ranges for the fermentation are typically between pH 5.0 to pH 9.0, where pH 6.0 to pH 8.0 is preferred. Fermentation may be performed under either aerobic or anaerobic conditions whereas aerobic conditions are generally preferred.

### Industrial Batch and Continuous Fermentations

Classical batch fermentation is a closed system where the composition of the medium is set at the beginning of the fermentation and not subject to artificial alterations during the fermentation. Thus, at the beginning of the fermentation the medium is inoculated with the desired organism or organisms, and fermentation is permitted to occur without adding anything to the system. Typically, a “batch” fermentation is batch with respect to the addition of carbon source and attempts are often made at controlling factors such as pH and oxygen concentration. In batch systems the metabolite and biomass compositions of the system change constantly up to the time the fermentation is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of end product or intermediate.

A variation on the standard batch system is the Fed-Batch system. Fed-Batch fermentation processes are also suitable in the present invention and comprise a typical batch system with the exception that the substrate is added in increments as the fermentation progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch fermentations are common and well known in the art and examples may be found in Thomas D. Brock in Biotechnology: A Textbook of Industrial Microbiology, Second Edition (1989) Sinauer Associates, Inc., Sunderland, Mass. (hereinafter “Brock”), or Deshpande, Mukund V., Appl. Biochem. Biotechnol., (1992) 36:227-234.

Although typically performed in batch mode, it is contemplated that the methods described herein would be adaptable to continuous fermentation method and may be practiced using either batch, fed-batch or continuous processes and that any known mode of fermentation would be suitable.

Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth.

Continuous fermentation allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to the medium being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra.

One of skill in the art will recognize that typically any amount, concentration, or other value or parameter that is given either as a range, preferred range, or a list of upper preferable values and lower preferable values, is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

### EXAMPLES

The present invention is further described in the following Examples. It should be understood that these Examples only illustrate the invention, which is defined only by the claims. The meaning of abbreviations used is as follows: “min” means minute(s), “h” means hour(s), “μL” means microliter(s), “mL” means milliliter(s), “L” means liter(s), “nm” means nanometer(s), “mm” means millimeter(s), “μm” means micrometer(s), “μM” means micromolar, “mM” means millimolar, “M” means molar, “mmol” means millimole(s), “μmol” means micromole(s), “μmol” means picomole(s), “g” means gram(s), “μg” means microgram(s), “mg” means milligram(s), “g” means the gravitation constant, “rpm” means revolutions per minute, and “cat#” means catalog number, “PN” means part number.

### General Methods

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or in Brock (supra). All reagents, restriction enzymes and materials used for the growth and maintenance of bacterial cells were obtained from BD Diagnostic Systems (Sparks, Md.), Invitrogen (Carlsbad, Calif.), Life Technologies (Rockville, Md.), QIAGEN (Valencia, Calif.) or Sigma-Aldrich Chemical Company (St. Louis, Mo.), unless otherwise specified.

### Peptide Expression System

The peptide expression system used was based on E. coli MG1655 (ATCC® 47076™)-derived strain QC1100 in combination with a pBAD-based expression vector. The modified E. coli MG1655 strain comprising a disruption in the endogenous araBAD operon is referred to herein as E. coli strain KK2000 (the nucleic acid sequence of an araB promoter is provided as SEQ ID NO: 19). A knockout of slyD (SEQ ID NOs: 20 and 21) was engineered into KK2000 to reduce background of LUMIO™-based in-cell labeling. KK2000 containing the slyD knockout is referred to herein as E. coli strain QC1100.

The peptides were expressed as fusions which were designed to include at least one region encoding an inclusion body tag (IBT) linked to a peptide of interest (POI). Appropriate restriction sites were included in the expression system to facilitate simple swapping of the DNA encoding the inclusion body tag and/or peptide of interest. The fusion peptide was designed to have a cleavable peptide linker (for example, an acid cleavable aspartic acid—protein moiety (DP)) between the inclusion body tag (IBT) and the peptide of interest (POI). Furthermore, the fusion peptide was also designed to include at least one tetracysteine tag (LUMIO™ tag; SEQ ID NO: 1) located on the C-terminus of the inclusion body tag wherein the tetracysteine tag was separated from the portion encoding the peptide of interest by the cleavable peptide linker.

The peptide expression plasmid pLR199 (SEQ ID NO: 2) used in this application contains a ColE1 type origin of replication, the bla gene to confer ampicillin resistance and the aadA-1 gene to confer spectinomycin (Spec) resistance (see co-pending U.S. patent application Ser. No. 12/1263,608 to Cheng et al., herein incorporated by reference). The tag/peptide fusion construct was driven by the pBAD promoter. The plasmid also encodes the gene for the araC regulator. The fusion peptide construct in pLR199 contains a small inclusion body tag IBT139 (SEQ ID NO: 3) and the tetracysteine tag CCPGCC (SEQ ID NO: 1) followed by a peptide of interest (such as peptide HC776124; SEQ ID NO: 4), creating fusion peptide IBT139-CCPGCC-HC776124 (SEQ ID NOs: 5 and 6). The QC1100 strain containing the pLR199 vector was referred as E. coli strain QC1101.

### FACS System Operating Conditions:

A Fluorescence Activated Cell Sorter (FACSVANTAGE™ SE-DiVa; Becton-Dickinson (BD Biosciences, Franklin Lakes, N.J.)) was configured with a single 488 nm argon ion laser (200 mW). The laser is used to induce light scattering by either the excitation of cellular fluorescent tags or the granularity within the cell. The SSC (Side Scatter Collector) light detection from the cell is collected through a microscope objective, transmitted via fiber light guide to an array of photo-multiplier tubes (PMTs). The FSC (Forward Scatter Collector) was constructed of a photo-diode. The SSC octagon configuration was composed of 5 PMTs in an octagon configuration. The LUMIO™ collection at 530 nm used a fluorescein isothiocyanate (FITC) filter (530 nm center, +/−15 nm bands) with a SSC filter of 488 nm bandpass (488 nm center, +/−10 nm bands). The system fluid used on the FACSVANTAGE™ SE-DiVa was FACSFLOW™ Sheath (Becton Dickinson) at an operating pressure of 28 psi (˜193 kPa) using a 70 μm diameter orifice tip.

The standard daily alignment of the instrument was performed using ALIGNFLOW™ (Molecular Probes, Inc., Eugene, Oreg.) 2.5 μm diameter fluorescent beads at an excitation/emission of 488 nm. The ALIGNFLOW™ beads were used as the daily alignment standard and the following instrument adjustments were made on the FACS to obtain the maximum PMT signal and minimum CV (coefficient of variation) for all channels on the instrument. The ALIGNFLOW™ beads were used to enable the daily adjustment of the FACS nozzle (X, Y, Z, α, and θ); in addition to the focus lens, channel height and channel height focus in all detector channels. The alignment of the FACS system can vary, but with the use of the ALIGNFLOW™ beads good alignment reproducibility was obtained. The ALIGNFLOW™ beads were either incorporated as a separate sample or directly into the sample to monitor the alignment and any potential instrument drift. The daily FACS alignment procedure, created in the DiVa Software (Becton Dickinson, v1.4), was performed and verified to within normal operating conditions.

The LUMIO™-stained cell samples were previously prepared in PBS (phosphate buffered saline) which is similar to the sheath fluid; therefore, no additional manipulation was needed for FACS analysis. Approximately 200 μL of a sample containing LUMIO™ stained cells was placed into a Falcon 12×75 mm, sterile polystyrene culture tube (Becton Dickinson) and into the instrument. The sample differential pressure was adjusted to obtain a stable 1000 events/second; at which, between 20,000 and 50,000 sample events were recorded. The variation, in sample recorded events, was due to the variation in cell concentration and limited sample volume. If the number of observed events was low, then the recorded events were then decreased. The samples scanned on the FACS for LUMIO™ analysis included, but were not limited to, an ALIGNFLOW™ bead sample, unstained LUMIO™ (negative control) and a series of LUMIO™ stained samples (experimental). The data obtained for the FACS samples included several different plot windows; which included dot plots for FSC-A vs SSC-A, FSC-A vs. FITC-A, SSC-A vs FITC-A and histograms for SSC-A, FSC-A, and FITC-A (width×height) for the particular channel (“A” is the computed area; “FS” is forward scatter; and “SS” is side scatter). During the recording of each sample, a gate was set on the FITC-A histogram between the 10³ and 10⁴ (log scale) to monitor and observe the sample LUMIO™ labeling efficiency. The recorded events within the gate on the FITC-A log scale provided a good indication of the sample LUMIO™ labeling efficiency. The recorded LUMIO™ sample data was saved and then within the DiVA software they were exported as FCS3 data files for further analysis.

### Example 1

### Construction of the Shotgun Expression Library of E. coli ATCC® 47076™

This example describes construction of a shotgun expression library of E. coli ATCC® 47076™ in a peptide production strain, which the produced fusion peptide contained the tetracysteine tag (CCPGCC; SEQ ID NO: 1) that allowed specific labeling of the fusion peptide.

A shot-gun library of random genomic fragments of E. coli MG1655 (ATCC® 47076™) was constructed on a broad-host-range vector pBHR1, which carries a kanamycin resistant marker and is compatible with the ColE1-based peptide expression plasmid. A set of multiple cloning sites (MCS) was first introduced into pBHR1 by annealing two oligonucleotides, RI linker Top ((5′-aattcgctagcgtcgacactagtc-3′; SEQ ID NO: 7) and RI linker Bot (5′-aattg actagtgtcgacgctagcg-3′; SEQ ID NO: 8), and cloned the annealed oligos into the EcoRI site of pBHR1. The resulting pBHR1 vector containing the MCS (SpeI-SalI-NheI-EcoRI) was designated as pDCQ601. The pDCQ601 vector was digested with SalI and partially filled in with dTTP and dCTP. The genomic DNA of E. coli ATCC® 47076™ was partially digested with Sau3AI to generate 1-3 kb fragments. The Sau3AI fragments were then partially filled in with dGTP and dATP, and ligated with the treated pDCQ601 vector. The ligation mixture was transformed into QC1101 containing peptide production plasmid pLR199. Approximately 20,000 kanamycin resistant and ampicillin resistant clones were obtained. Thirty-six of those were randomly picked and about 90% of them contained inserts about 1-3 kb in size. The clones were pooled and this library was designated as “QC1300 library”. The strain of the vector pDCQ601 transformed into QC1101 was designated as the control QC1311.

### Example 2

### Sorting of QC1300 Library by FACS

QC1300 library clones contained the peptide production plasmid pLR199, which has a tetracysteine tag (CCPGCC, SEQ ID NO: 1) inserted into the fusion peptide IBT139-HC776124 to form fusion peptide IBT139-CCPGCC-HC776124 (SEQ ID NOs: 5 and 6) (see co-pending U.S. patent application Ser. No. 11/782,836). The fusion peptide was produced as inclusion bodies in E. coli via the inclusion body promoting sequence IBT139 (SEQ ID NO: 3) fused at the N-terminus of the peptide of interest HC776124 (SEQ ID NO: 4). Specific labeling of the fusion peptide could be achieved by biarsenical ligands binding to tetracysteine tag. The fluorescein derivative with two As(III) substituents, FlAsH-EDT₂ (LUMIO™ Green), only fluoresces after the arsenics bind to the cysteine thiols in the target fusion peptide. The LUMIO™ reagents were obtained from Invitrogen (Carlsbad, Calif.).

The QC1300 library cells were labeled using TC-FlAsH™ In-Cell tetracysteine tag detection kit (Invitrogen). The library cells were thawed from frozen stocks and grew in 20 mL LB with ampicillin (100 μg/mL) and kanamycin (50 μg/mL) till an OD₆₀₀ about 0.5-0.7. The cells were then induced with 0.2% L-arabinose for about 3 hours. The induced cells were diluted and normalized to an OD₆₀₀ of 0.1. Approximately 3×10⁷ cells were then labeled with 20 μM FlAsH reagent for 2 hours at room temperature (˜22° C.) in the dark. The labeled cells were washed twice with BAL wash buffer and resuspended in PBS for sorting on FACS based on fluorescence. The gate for the first sort was set for the top 10% of fluorescent cells. About 100,000 events were collected and plated on LB plates with ampicillin (100 μg/mL) and kanamycin (50 μg/mL). The plates were incubated at 37° C. overnight. The colonies grown on the plates were pooled and aliquotes were used to grow cells for the next round of labeling following the same protocol as described above. The parameters used for each round of sorting is shown in Table 1.

TABLE 1
Parameter used for sorting Library QC1300

| | | Percent Sort of Previous Round |
|---|---|---|
| Round No. | Number of Events | (%) |
| QC1300^{a} | NA | NA |
| Library | | |
| 1 | 100,000 | 10% |
| 2 | 50,000 | 5% |
| 3 | 50,000 | 0.5% |
| 4 | 10,000 | 0.5% |
| 5 | 10,000 | 0.5% |
| 6 | 5,000 | 0.1% |

^{a}= Library QC1300 titer >20,000.

A total of six rounds was performed and aliquotes from each round were also frozen. The mean fluorescence of the initial library was lower than the control. After several rounds of sorting, the mean fluorescence of sorted population shifted higher than that of the control. During the different rounds of sorting, the fluorescence of the internal bead standard was superimposable.

### Example 3

### Sequencing and Confirmation of Genes in the Sorted Clones

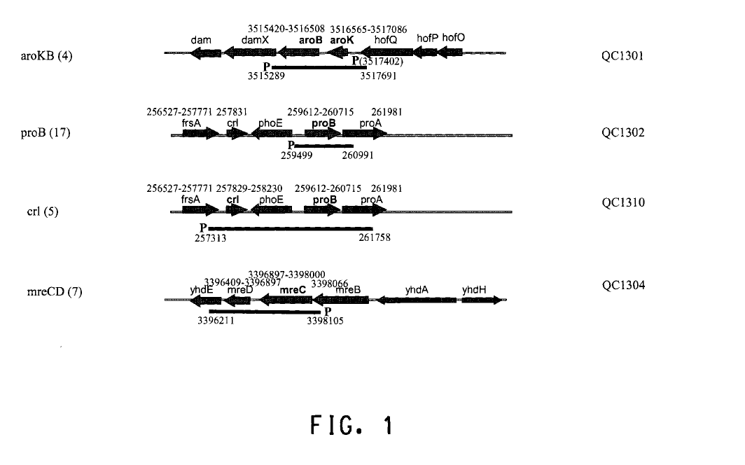

Several hundred colonies obtained from round 4 and round 6 sortings (Table 1) were screened by PCR first for the presence of the insert on the library plasmids. The clones containing the insert were then sequenced from both ends of the insert to map the insert fragment to the E. coli genome (GENBANK®Accession No. 000096; the complete genome sequence of Escherichia coli strain K-12 substrain MG1655). Among approximately 200 sequenced clones, QC1301 was isolated 4 times which contained the intact aroKB genes with its native promoter. QC1302 was isolated 17 times which contained the intact proB gene and partial proA gene. QC1310 was isolated 5 times which contained the crl gene and the proB gene. QC1304 was isolated 7 times which contained the intact mreCD genes. The genetic organization of the regions and the exact location of the fragments contained on the plasmids were shown in FIG. 1. The sequences of several of the identified genes is provided in Table 2.

TABLE 2
Sequence information for several of the identified genes.

| | Coding Sequence | Amino Acid |
|---|---|---|
| Gene Name | (SEQ ID NO:) | (SEQ ID NO:) |
| aroB | 9 | 10 |
| aroK | 11 | 12 |
| proB | 13 | 14 |
| crl | 15 | 16 |
| mreC | 17 | 18 |

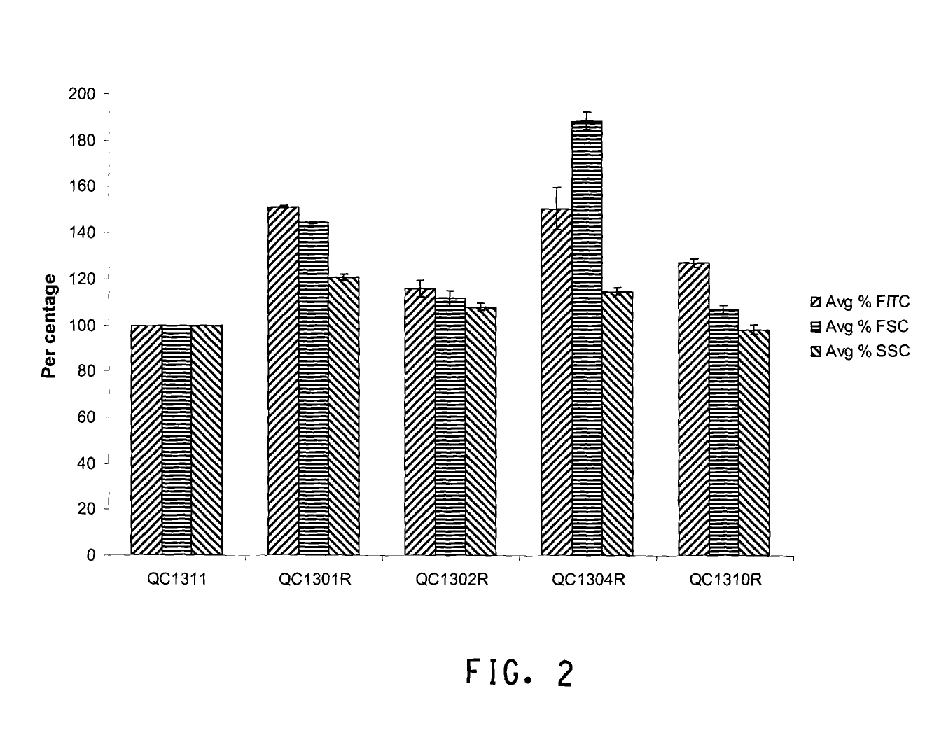

To verify that the genes contained on the plasmids contributed to the phenotypes, the plasmids were isolated from each strain and retransformed into a fresh host. The retransformed clean strains (QC1301R, QC1302R, QC1304R and QC1310R, respectively) were induced and labeled for FACS analysis and in-gel analysis (see Example 4). Triplicates of each of the E. coli strains (QC1311, QC1301R, QC1302R, QC1304R and QC1310R) were grown in 3 mL LB with ampicillin (100 μg/mL) and kanamycin (50 μg/mL) till an OD₆₀₀ of about 0.5-0.7. The cells were then induced with 0.2% L-arabinose for about 3 hours. The induced cells were labeled with 20 μM FlAsH™ reagent for 1.5 hours at room temperature (˜22° C.) in the dark. The labeled cells were washed twice with BAL wash buffer and resuspended in PBS. FACS analysis of these strains is shown in FIG. 2. Error bars represent the standard deviations of the triplicate cultures. QC1301R and QC1304R showed about 50-60% higher fluorescence than the control (QC1311). QC1302R and QC1310R showed about 20-30% higher fluorescence than the control. For forward scattering (FSC), which usually reflects cell size and cell shape, QC1301R showed about 45% higher and QC1304 showed about 90% higher than the control. QC1302R and QC1310R showed only about 10% higher than the control. For side scattering (SSC), which usually reflects intracellular granularity, QC1301R and QC1304R showed about 10-20% higher, while QC1302R showed less than 10% higher than the control. QC1310R did not show higher SSC than the control. All 4 retransformed strains showed higher fluorescence than the control, which could explain that they were isolated from FACS sorting for higher fluorescence attributed to the plasmid-encoded genes.

### Example 4

### Peptide Quantitation by In-gel LUMIO™ Analysis

The fusion peptide containing the tetracysteine tag could be specifically labeled with the LUMIO™ Green detection reagent (FlAsH-EDT₂) using the LUMIO™ Green detection kit (Invitrogen). This is the in-gel (in vitro) labeling, which completely lyses the cells and labels the tagged protein in the cell extract. The labeled protein on the gel could be visualized under UV light. The intensity of the labeled protein band could be quantified by image analysis. The linear range of the fluorescence images of the system was established using different amounts of the same labeling reaction mixture.

Triplicates of each of the E. coli strains (QC1311, QC1301R, QC1302R, QC1304R and QC1310R) were grown in 3 mL LB with ampicillin (100 μg/mL) and kanamycin (50 μg/mL) till an OD₆₀₀ of about 0.5-0.7. The cells were then induced with 0.2% L-arabinose for about 3 hours. Final OD₆₀₀ values of the cultures were recorded. Aliquotes of cells were diluted for in-cell labeling for FACS analysis as described in Example 3. In parallel, the same volume of the end point cultures were spun down and frozen for in-gel analysis. The pellets were lysed with B-PER® (Bacterial Protein Extraction Reagent) lysis buffer (Pierce Chemical Co., Rockford, Ill.). The whole cell lysate was labeled using the LUMIO™ Green detection kit (Invitrogen) following the manufacture's instructions. The stained lysate was run on a NUPAGE® 4-12% Bis-Tris gel with MES running buffer (Invitrogen). The BENCHMARK™ fluorescent protein standard (Invitrogen) was used. The gel was visualized under UV light.

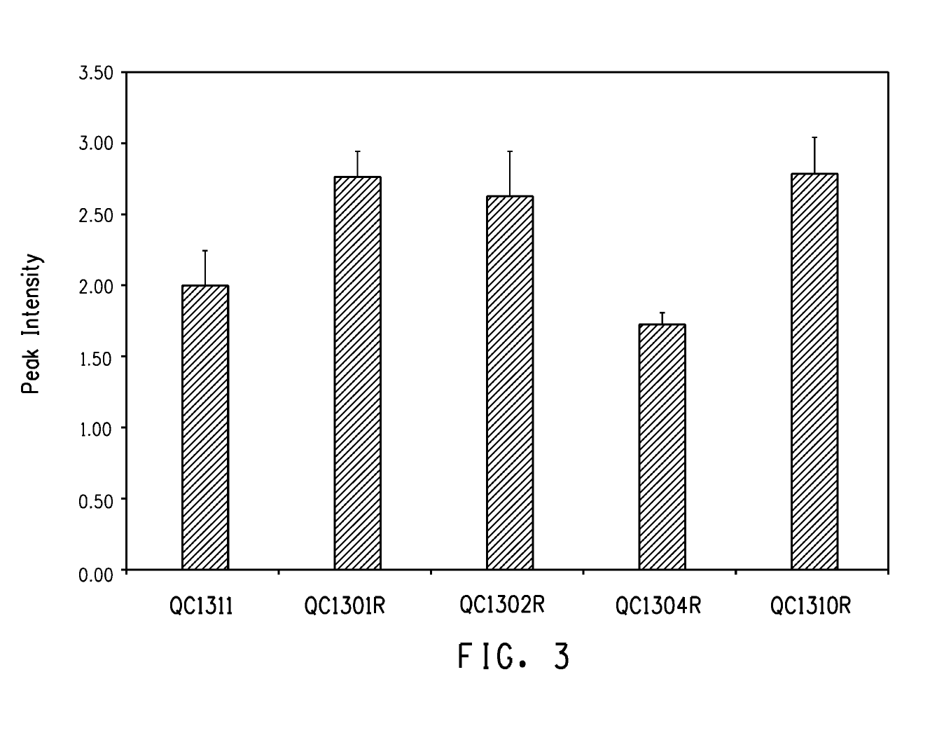

After taking a picture of the gel, the gel was rinsed, stained with SIMPLYBLUE™ (Invitrogen) and destained with deionized water. The intensity of the fusion peptide band was quantified using ImageJ software (available from Rasband, W. S. Research Services Branch, National Institute of Mental Health, Bethesda, Md., USA and Abramoff, M. D., et al., (2004). Biophotonics International, volume 11, issue 7, pp. 36-42). The fluorescent intensity of each band was then normalized by the final OD₆₀₀ of the cultures (FIG. 3). Results showed that QC1301R, QC1302R and QC1310R were about 30-40% higher than the control, whereas QC1304R was slightly lower than the control. The QC1304R data might be complicated by defects in cell division, as it was reported that introduction of the mre genes on multicopy plasmid leads to cell filamentation phenotype (Lee, et al., (2003), Current Microbiology 47:146).

### Example 5

### Peptide Quantitation by Bioanalyzer

To validate the in-gel peptide quantitation by fluorescence image analysis, the peptide amount in the QC1301R and the control QC1311R was also quantified by the Bioanalyzer method without LUMIO™ labeling.

QC1301R and the control QC1311R cells were grown in 20 mL LB with ampicillin (100 μg/mL) and kanamycin (50 μg/mL) at 37° C. After about 2 hours, they reached an OD₆₀₀ of 0.6. The cells were then induced with 0.2% L-arabinose and continued growing. At 5 hour (3 hours after induction) and 6 hour (4 hour after induction) points, aliquotes of samples were taken from each flask and were normalized to an OD₆₀₀ of 1. Same volume (1-mL) of the OD₆₀₀ 1 cells were pelleted and the cell pellets were used for peptide quantification by Agilent Bioanalyzer 2100 (Agilent Technologies, Palo Alto, Calif.). The cell pellets were resuspended in lysis solution containing 0.1 mg/mL lysozyme for lysis as well as the internal standard. The samples and the ladder were prepared for loading according to the users' guide for the Protein 80 chip (Agilent Technologies). The chip was set up and run following the manufacturer's instructions. The calculated fusion protein (FP) titer and specific FP productivity from triplicates were shown in Table 3. The specific FP productivity was calculated assuming 1 OD₆₀₀ of cells yields 300 mg dry cell weight per liter. Results showed that in the same 1 mL of OD₆₀₀ 1 cells, QC1301R produced about 50% more fusion peptide than the control at 5 hour time point. This was similar to the results in Example 4 from the in-gel fluorescence quantitation. At later time point (6 hour), the fusion peptide titer in QC1301R remained about the same, but the peptide titer in QC1311R decreased with time.

TABLE 3
Bioanalyzer analysis of QC1301R and QC1311R cells.

| | OD | Time | avg mg | std. dev | avg | std. dev |
|---|---|---|---|---|---|---|
| run # | (600 nm) | (hr) | FP/L | mg FP/L | Y_{FP/X} | Y_{FP/X} |
| QC1301R | 1 | 5 | 47.3 | 1.2 | 157.6 | 3.9 |
| QC1301R | 1 | 6 | 45.3 | 3.7 | 150.9 | 12.5 |
| QC1311R | 1 | 5 | 31.5 | 1.9 | 104.9 | 6.2 |
| QC1311R | 1 | 6 | 24.2 | 0.4 | 80.5 | 1.2 |

### Example 6

### Analysis of Transcripts Level by RT-PCR

To confirm that mRNA transcript level of the genes in these strains was increased from multicopy plasmid expression, real time reverse transcription-PCR (RT-PCR) analysis was performed. Samples were grown in 20 mL LB with ampicillin (100 μg/mL) and kanamycin (50 μg/mL) at 37° C. for about 2 hours till they reached an OD₆₀₀ of 0.5-0.7. An aliquot of 1 mL culture was taken as the pre-induction (t=0) sample and immediately mixed with 2 mL of RNAprotect reagent (Qiagen, Valencia, Calif.). The cells were then induced with 0.2% L-arabinose for about 3 hours. An aliquot sample was taken every 15 min till 60 min after induction. The total RNA samples were prepared using RNeasy mini kit from Qiagen. Real Time RT-PCR was performed on an Applied Biosystems 7900 Sequence Detection System instrument using a two-step method (Applied Biosystems, Foster City, Calif.). In step 1, cDNA was made from the provided RNA samples. The samples were first treated with DNase (Qiagen) for 15 min at room temperature followed by inactivation for 5 min at 75° C., to eliminate any residual genomic DNA. The RNA was then reverse transcribed using the High Capacity cDNA Reverse Transcription Kit from Applied Biosystems according to the manufacturer's recommended protocol. In step 2, the PCR reaction (20 μL) was run on the ABI 7900 using the following reagents: 10 μL ABI TAQMAN® Universal PCR Master Mix without UNG (uracil N-glycosylase; PN 4326614), 0.2 μL each forward and reverse primers (100 μM), 0.05 μL TAQMAN® probe (100 μM), 2 μL RNA and 7.55 μL RNase free water. The PCR primers and dual labeled TAQMAN® probes were designed using Primer Express v2.0 software from Applied Biosystems and were purchased from Sigma-Genosys. The primer and probe sequences are shown in Table 4.

TABLE 4
Primer and Prober Sequences.

| | | | Sequence |
|---|---|---|---|
| | | | (5′ to 3′) |
| Gene | Primer Name | Direction | (SEQ ID NO:) |
| 16S rRNA | 16s-518F | Forward | CCAGCAGCCGCGGTAAT |
| E. coli | | | (SEQ ID NO: 22) |
| | 16s-579R | Reverse | TGCGCTTTACGCCCAGTAAT |
| | | | (SEQ ID NO: 23) |
| | 16s-536T | Probe | CGGAGGGTGCAAGCGTTAAT |
| | | | CGG |
| | | | (SEQ ID NO: 24) |
| E. coli | aroB-158F | Forward | TCCGCGGCGTACTTGAA |
| aroB | | | (SEQ ID NO: 25) |
| | aroB-218R | Reverse | TCGCCGTCAGGGAGGAT |
| | | | (SEQ ID NO: 26) |
| | aroB-176T | Probe | AGGCGGGTGTTAACGTCGAT |
| | | | AGCGT |
| | | | (SEQ ID NO: 27) |
| E. coli | proB-745F | Forward | ATTTCCGTCGGTACGCTGTT |
| proB | | | (SEQ ID NO: 28) |
| | proB-808R | Reverse | AAATCCAGCGTTTACGGTT |
| | | | TTC |
| | | | (SEQ ID NO: 29) |
| | proB-766T | Probe | CATGCCCAGGCGACTCCGC |
| | | | (SEQ ID NO: 30) |
| E. coli | crl-117F | Forward | TGTATGCGTCAACGTGAAA |
| crl | | | CC |
| | | | (SEQ ID NO: 31) |
| | crl-186R | Reverse | CGCTTCCAGCTCCATCCA |
| | | | (SEQ ID NO: 32) |
| | crl-140T | Probe | CACCGGAAGTGCGTGAATT |
| | | | CTGGG |
| | | | (SEQ ID NO: 33) |

The following real time PCR thermal cycling conditions were used: 10 minutes at 95° C. followed by 40 cycles of 95° C. for 15 seconds and 60° C. for 1 minute. The samples were run at a concentration of 1 ng cDNA/r×n. A (−) reverse transcription RNA control of each sample was run with each primer set to confirm the absence of genomic DNA. All reactions were run in triplicate.

The relative quantitation in the samples was calculated using the ΔΔCt method (see Applied Biosystems User Bulletin #2 “Relative Quantitation of Gene Expression”, Dec. 11, 1997, updated October 2001). A linear regression was performed for each primer and probe set and the efficiencies were within 90-100%. The 16S rRNA gene was used to normalize the quantitation of the target gene for differences in the amount of total RNA added to each reaction. The relative quantitation (RQ) value is the fold increase in expression of the gene in the samples relative to the control T=0 calibrator sample. The aroB expression in QC1301R showed about 6-fold higher than the control. The proB expression in QC1302R showed about 50-fold higher than the control. The crl expression in QC1310R showed about 40-fold higher than the control. The plasmid in QC1310R contained both the crl gene and the proB gene. The proB expression in QC1310R showed about 10-fold higher than the control. Results confirmed that the mRNA transcripts levels of the respective genes were increased in these strains.

What is claimed is:

1. A method of producing a peptide of interest comprising:
a) providing a recombinant Escherichia coli host cell comprising
i) at least one chimeric genetic construct encoding a peptide of interest;
ii) a genetic modification that increases expression of at least one endogenous gene selected from the group consisting of the aroB, aroK, proB, crl, and combinations thereof.
b) growing the recombinant Escherichia coli host cell of (a) whereby the peptide of interest is produced;
c) optionally recovering the peptide of interest produced in step (b).

2. The method of claim 1, wherein the genetic modification is an increase in the copy number of the endogenous gene.

3. The method of claim 1, wherein the peptide of interest is a single chain peptide of 14 to 600 amino acids in length.

4. The method of claim 1, wherein the recombinant Escherichia coli host cell further comprising down-regulated expression of an endogenous gene or operon selected from the group consisting of araBAD operon, slyD gene, and a combination of these.

5. The method of claim 3, wherein the peptide of interest is expressed as part of a fusion peptide comprising the general structure:
IBT-CL-POI
or
POI-CL-IBT
wherein;
IBT means at least one inclusion body tag;
CL means at least one cleavable peptide linker; and
POI means at least one peptide of interest.

6. A recombinant Escherichia coli cell comprising:
i) at least one chimeric genetic construct encoding a peptide of interest; and
ii) a genetic modification that increases expression of at least one endogenous gene selected from the group consisting of the aroB, aroK, proB, crl, and a combination of these.

7. The recombinant host cell of claim 6, wherein the genetic modification is an increase in the copy number of the endogenous gene.

8. The recombinant host cell of claim 6, wherein the peptide of interest is a single chain peptide of 14 to 600 amino acids in length.

9. The recombinant Escherichia coli cell of claim 6, further comprising a disruption in a chromosomal copy of an endogenous gene or operon selected from the group consisting of araBAD operon, slyD gene, and a combination of these.

10. The recombinant Escherichia coli cell of claim 8, wherein the peptide of interest is expressed as part of a fusion peptide comprising the general structure:
IBT-CL-POI
or
POI-CL-IBT
wherein;
IBT means at least one inclusion body tag;
CL means at least one cleavable peptide linker; and
POI means at least one peptide of interest.

11. The recombinant host cell of claim 8, wherein the peptide of interest has affinity for a body surface selected from the group consisting of hair, skin, nail, tooth, and tooth pellicle.


SEQUENCE LISTING (481 sequences)

SEQ ID NO: 1 - protein, 6 aa, artificial sequence
  Synthetic construct - tetracysteine tag
CCPGCC

SEQ ID NO: 2 - DNA, 6034 bp, artificial sequence
  plasmid
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcttacctgacgctttttatcgcaactctctactgtttctccatacccgttttttgggctaacaggaggaattacatatgcagcagcgtttccagtggcagttcgaacagcagccgcgtggtcagcagcgtttccagtggcagttcgaacagcagccgcgtggtcagcagcgtttccagtggcagttcgaacagcagccggaaggtcagcagcgtttccagtggcagttcgaacagcagggatcttgctgtccgggctgttgcggatccgaccctggcattccgtggtggaacattcgtgctcctctgaatgcaggtgcgggcatcccttggtggaatattcgtgctccgctgaacgccggtggttccggtccgggtagcggtggtaatacttctcagctgtccacgggtggcggtaacactagccagctgagcacgggcggccctaaaaagccgggcgacccgggtattccgtggtggaatatccgtgccccgctgaacgcaggtgccggcatcccgtggtggaacattcgtgcacctctgaatgctggtggttccggtccaggctctggcggcaacacttcccagctgtccaccggcggtggcaacaccagccagctgtctactggtggtccgaagaaaccgggtgactaataaggcgcgccgacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 3 - protein, 53 aa, artificial sequence
  synthetic construct
QQRFQWQFEQQPRGQQRFQWQFEQQPRGQQRFQWQFEQQPEGQQRFQWQFEQQ

SEQ ID NO: 4 - protein, 129 aa, artificial sequence
  Synthetic construct. Multi-block hair-binding peptide
GSDPGIPWWNIRAPLNAGAGIPWWNIRAPLNAGGSGPGSGGNTSQLSTGGGNTSQLSTGGPKKPGDPGIPWWNIRAPLNAGAGIPWWNIRAPLNAGGSGPGSGGNTSQLSTGGGNTSQLSTGGPKKPGD

SEQ ID NO: 5 - DNA, 579 bp, artificial sequence
  Synthetic construct - chimeric gene encoding fusion peptide IBT139.CCPGCC-HC776124
atgcagcagcgtttccagtggcagttcgaacagcagccgcgtggtcagcagcgtttccagtggcagttcgaacagcagccgcgtggtcagcagcgtttccagtggcagttcgaacagcagccggaaggtcagcagcgtttccagtggcagttcgaacagcagggatcttgctgtccgggctgttgcggatccgaccctggcattccgtggtggaacattcgtgctcctctgaatgcaggtgcgggcatcccttggtggaatattcgtgctccgctgaacgccggtggttccggtccgggtagcggtggtaatacttctcagctgtccacgggtggcggtaacactagccagctgagcacgggcggccctaaaaagccgggcgacccgggtattccgtggtggaatatccgtgccccgctgaacgcaggtgccggcatcccgtggtggaacattcgtgcacctctgaatgctggtggttccggtccaggctctggcggcaacacttcccagctgtccaccggcggtggcaacaccagccagctgtctactggtggtccgaagaaaccgggtgactaataa

SEQ ID NO: 6 - protein, 190 aa, artificial sequence
  Synthetic construct - fusion peptide IBT139.CCPGCC-HC776124
QQRFQWQFEQQPRGQQRFQWQFEQQPRGQQRFQWQFEQQPEGQQRFQWQFEQQGSCCPGCCGSDPGIPWWNIRAPLNAGAGIPWWNIRAPLNAGGSGPGSGGNTSQLSTGGGNTSQLSTGGPKKPGDPGIPWWNIRAPLNAGAGIPWWNIRAPLNAGGSGPGSGGNTSQLSTGGGNTSQLSTGGPKKPGD

SEQ ID NO: 7 - DNA, 24 bp, artificial sequence
  synthetic construct
aattcgctagcgtcgacactagtc

SEQ ID NO: 8 - DNA, 19 bp, artificial sequence
  synthetic construct
actagtgtcgacgctagcg

SEQ ID NO: 9 - DNA, 1089 bp, Escherichia coli
atggagaggattgtcgttactctcggggaacgtagttacccaattaccatcgcatctggtttgtttaatgaaccagcttcattcttaccgctgaaatcgggcgagcaggtcatgttggtcaccaacgaaaccctggctcctctgtatctcgataaggtccgcggcgtacttgaacaggcgggtgttaacgtcgatagcgttatcctccctgacggcgagcagtataaaagcctggctgtactcgataccgtctttacggcgttgttacaaaaaccgcatggtcgcgatactacgctggtggcgcttggcggcggcgtagtgggcgatctgaccggcttcgcggcggcgagttatcagcgcggtgtccgtttcattcaagtcccgacgacgttactgtcgcaggtcgattcctccgttggcggcaaaactgcggtcaaccatcccctcggtaaaaacatgattggcgcgttctaccaacctgcttcagtggtggtggatctcgactgtctgaaaacgcttcccccgcgtgagttagcgtcggggctggcagaagtcatcaaatacggcattattcttgacggtgcgttttttaactggctggaagagaatctggatgcgttgttgcgtctggacggtccggcaatggcgtactgtattcgccgttgttgtgaactgaaggcagaagttgtcgccgccgacgagcgcgaaaccgggttacgtgctttactgaatctgggacacacctttggtcatgccattgaagctgaaatggggtatggcaattggttacatggtgaagcggtcgctgcgggtatggtgatggcggcgcggacgtcggaacgtctcgggcagtttagttctgccgaaacgcagcgtattataaccctgctcaagcgggctgggttaccggtcaatgggccgcgcgaaatgtccgcgcaggcgtatttaccgcatatgctgcgtgacaagaaagtccttgcgggagagatgcgcttaattcttccgttggcaattggtaagagtgaagttcgcagcggcgtttcgcacgagcttgttcttaacgccattgccgattgtcaatcagcgtaa

SEQ ID NO: 10 - protein, 362 aa, Escherichia coli
MERIVVTLGERSYPITIASGLFNEPASFLPLKSGEQVMLVTNETLAPLYLDKVRGVLEQAGVNVDSVILPDGEQYKSLAVLDTVFTALLQKPHGRDTTLVALGGGVVGDLTGFAAASYQRGVRFIQVPTTLLSQVDSSVGGKTAVNHPLGKNMIGAFYQPASVVVDLDCLKTLPPRELASGLAEVIKYGIILDGAFFNWLEENLDALLRLDGPAMAYCIRRCCELKAEVVAADERETGLRALLNLGHTFGHAIEAEMGYGNWLHGEAVAAGMVMAARTSERLGQFSSAETQRIITLLKRAGLPVNGPREMSAQAYLPHMLRDKKVLAGEMRLILPLAIGKSEVRSGVSHELVLNAIADCQSA

SEQ ID NO: 11 - DNA, 522 bp, Escherichia coli
atggcagagaaacgcaatatctttctggttgggcctatgggtgccggaaaaagcactattgggcgccagttagctcaacaactcaatatggaattttacgattccgatcaagagattgagaaacgaaccggagctgatgtgggctgggttttcgatttagaaggcgaagaaggcttccgcgatcgcgaagaaaaggtcatcaatgagttgaccgagaaacagggtattgtgctggctactggcggcggctctgtgaaatcccgtgaaacgcgtaaccgtctttccgctcgtggcgttgtcgtttatcttgaaacgaccatcgaaaagcaacttgcacgcacgcagcgtgataaaaaacgcccgttgctgcacgttgaaacaccgccgcgtgaagttctggaagcgttggccaatgaacgcaatccgctgtatgaagagattgccgacgtgaccattcgtactgatgatcaaagcgctaaagtggttgcaaaccagattattcacatgctggaaagcaactaa

SEQ ID NO: 12 - protein, 173 aa, Escherichia coli
MAEKRNIFLVGPMGAGKSTIGRQLAQQLNMEFYDSDQEIEKRTGADVGWVFDLEGEEGFRDREEKVINELTEKQGIVLATGGGSVKSRETRNRLSARGVVVYLETTIEKQLARTQRDKKRPLLHVETPPREVLEALANERNPLYEEIADVTIRTDDQSAKVVANQIIHMLESN

SEQ ID NO: 13 - DNA, 1104 bp, Escherichia coli
atgagtgacagccagacgctggtggtaaaactcggcaccagtgtgctaacaggcggatcgcgccgtctgaaccgtgcccatatcgttgaacttgttcgccagtgcgcgcagttacatgccgccgggcatcggattgttattgtgacgtcgggcgcgatcgccgccggacgtgagcacctgggttacccggaactgccagcgaccatcgcctcgaaacaactgctggcggcggtagggcagagtcgactgattcaactgtgggaacagctgttttcgatttatggcattcacgtcgggcaaatgctgctgacccgtgctgatatggaagaccgtgaacgcttcctgaacgcccgcgacaccctgcgagcgttgctcgataacaatatcgttccggtaatcaatgagaacgatgctgtcgctacggcagagattaaggtcggcgataacgataacctttctgcgctggcggcgattcttgcgggtgccgataaactgttgctgctgaccgatcaaaaaggtttgtataccgctgacccgcgcagcaatccgcaggcagaactgattaaagatgtttacggcattgatgacgcactgcgcgcgattgccggtgacagcgtttcaggcctcggaactggcggcatgagtaccaaattgcaggccgctgacgtggcttgccgtgcgggtatcgacaccattattgccgcgggcagcaagccgggcgttattggtgatgtgatggaaggcatttccgtcggtacgctgttccatgcccaggcgactccgcttgaaaaccgtaaacgctggattttcggtgcgccgccggcgggtgaaatcacggtagatgaaggggcaactgccgccattctggaacgcggcagctccctgttgccgaaaggcattaaaagcgtgactggcaatttctcgcgtggtgaagtcatccgcatttgcaacctcgaaggccgcgatatcgcccacggcgtcagtcgttacaacagcgatgcattacgccgtattgccggacaccactcgcaagaaattgatgcaatactgggatatgaatacggcccggttgccgttcaccgtgatgacatgattacccgttaa

SEQ ID NO: 14 - protein, 367 aa, Escherichia coli
MSDSQTLVVKLGTSVLTGGSRRLNRAHIVELVRQCAQLHAAGHRIVIVTSGAIAAGREHLGYPELPATIASKQLLAAVGQSRLIQLWEQLFSIYGIHVGQMLLTRADMEDRERFLNARDTLRALLDNNIVPVINENDAVATAEIKVGDNDNLSALAAILAGADKLLLLTDQKGLYTADPRSNPQAELIKDVYGIDDALRAIAGDSVSGLGTGGMSTKLQAADVACRAGIDTIIAAGSKPGVIGDVMEGISVGTLFHAQATPLENRKRWIFGAPPAGEITVDEGATAAILERGSSLLPKGIKSVTGNFSRGEVIRICNLEGRDIAHGVSRYNSDALRRIAGHHSQEIDAILGYEYGPVAVHRDDMITR

SEQ ID NO: 15 - DNA, 402 bp, Escherichia coli
atgacgttaccgagtggacacccgaagagcagattgatcaaaaaatttaccgcactaggcccgtatattcgtgaaggtaagtgcaaagataatcgattctttttcgattgtctggctgtatgcgtcaacgtgaaaccggcaccggaagtgcgtgaattctggggctggtggatggagctggaagcgcaggaatcccgttttacctacagttaccagtttggtctgttcgataaagcaggcgactggaagagtgttccggtaaaagacactgaagtggttgaacgactggagcacaccctgcgtgagtttcacgagaagctgcgtgaactgctgacgacgctgaatctgaagctggaaccggcggatgattttcgtgacgagccggtgaagttaacggcgtga

SEQ ID NO: 16 - protein, 133 aa, Escherichia coli
MTLPSGHPKSRLIKKFTALGPYIREGKCKDNRFFFDCLAVCVNVKPAPEVREFWGWWMELEAQESRFTYSYQFGLFDKAGDWKSVPVKDTEVVERLEHTLREFHEKLRELLTTLNLKLEPADDFRDEPVKLTA

SEQ ID NO: 17 - DNA, 1104 bp, Escherichia coli
atgaagccaatttttagccgtggcccgtcgctacagattcgccttattctggcggtgctggtggcgctcggcattattattgccgacagccgcctggggacgttcagtcaaatccgtacttatatggataccgccgtcagtcctttctactttgtttccaatgctcctcgtgaattgctggatggcgtatcgcagacgctggcctcgcgtgaccaattagaacttgaaaaccgggcgttacgtcaggaactgttgctgaaaaacagtgaactgctgatgcttggacaatacaaacaggagaacgcgcgtctgcgcgagctgctgggttccccgctgcgtcaggatgagcagaaaatggtgactcaggttatctccacggttaacgatccttatagcgatcaagttgttatcgataaaggtagcgttaatggcgtttatgaaggccagccggtcatcagcgacaaaggtgttgttggtcaggtggtggccgtcgctaaactgaccagtcgcgtgctgctgatttgtgatgcgacccacgcgctgccaatccaggtgctgcgcaacgatatccgcgtaattgcagccggtaacggttgtacggatgatttgcagcttgagcatctgccggcgaatacggatattcgtgttggtgatgtgctggtgacttccggtctgggcggtcgtttcccggaaggctatccggtcgcggttgtctcttccgtaaaactcgatacccagcgcgcttatactgtgattcaggcgcgtccgactgcagggctgcaacgtttgcgttatctgctgctgctgtggggggcagatcgtaacggcgctaacccgatgacgccggaagaggtgcatcgtgttgctaatgaacgtctgatgcagatgatgccgcaggtattgccttcgccagacgcgatggggccaaagttacctgaaccggcaacggggatcgctcagccgactccgcagcaaccggcgacaggaaatgcagctactgcgcctgctgcgccgacacagcctgctgctaatcgctctccacaaagggctacgccgccgcaaagtggtgctcaaccgcctgcgcgtgcgccgggagggcaatag

SEQ ID NO: 18 - protein, 367 aa, Escherichia coli
MKPIFSRGPSLQIRLILAVLVALGIIIADSRLGTFSQIRTYMDTAVSPFYFVSNAPRELLDGVSQTLASRDQLELENRALRQELLLKNSELLMLGQYKQENARLRELLGSPLRQDEQKMVTQVISTVNDPYSDQVVIDKGSVNGVYEGQPVISDKGVVGQVVAVAKLTSRVLLICDATHALPIQVLRNDIRVIAAGNGCTDDLQLEHLPANTDIRVGDVLVTSGLGGRFPEGYPVAVVSSVKLDTQRAYTVIQARPTAGLQRLRYLLLLWGADRNGANPMTPEEVHRVANERLMQMMPQVLPSPDAMGPKLPEPATGIAQPTPQQPATGNAATAPAAPTQPAANRSPQRATPPQSGAQPPARAPGGQ

SEQ ID NO: 19 - DNA, 100 bp, Escherichia coli
tttttatccataagattagcggatcctacctgacgctttttatcgcaactctctactgtttctccatacccgttttttgggctaacaggaggaattaacc

SEQ ID NO: 20 - DNA, 591 bp, Escherichia coli
atgaaagtagcaaaagacctggtggtcagcctggcctatcaggtacgtacagaagacggtgtgttggttgatgagtctccggtgagtgcgccgctggactacctgcatggtcacggttccctgatctctggcctggaaacggcgctggaaggtcatgaagttggcgacaaatttgatgtcgctgttggcgcgaacgacgcttacggtcagtacgacgaaaacctggtgcaacgtgttcctaaagacgtatttatgggcgttgatgaactgcaggtaggtatgcgtttcctggctgaaaccgaccagggtccggtaccggttgaaatcactgcggttgaagacgatcacgtcgtggttgatggtaaccacatgctggccggtcagaacctgaaattcaacgttgaagttgtggcgattcgcgaagcgactgaagaagaactggctcatggtcacgttcacggcgcgcacgatcaccaccacgatcacgaccacgacggttgctgcggcggtcatggccacgatcacggtcatgaacacggtggcgaaggctgctgtggcggtaaaggcaacggcggttgcggttgccactaa

SEQ ID NO: 21 - protein, 196 aa, Escherichia coli
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGCCGGHGHDHGHEHGGEGCCGGKGNGGCGCH

SEQ ID NO: 22 - DNA, 17 bp, artificial sequence
  primer
ccagcagccgcggtaat

SEQ ID NO: 23 - DNA, 20 bp, artificial sequence
  primer
tgcgctttacgcccagtaat

SEQ ID NO: 24 - DNA, 23 bp, artificial sequence
  primer
cggagggtgcaagcgttaatcgg

SEQ ID NO: 25 - DNA, 17 bp, artificial sequence
  primer
tccgcggcgtacttgaa

SEQ ID NO: 26 - DNA, 17 bp, artificial sequence
  primer
tcgccgtcagggaggat

SEQ ID NO: 27 - DNA, 25 bp, artificial sequence
  primer
aggcgggtgttaacgtcgatagcgt

SEQ ID NO: 28 - DNA, 20 bp, artificial sequence
  primer
atttccgtcggtacgctgtt

SEQ ID NO: 29 - DNA, 22 bp, artificial sequence
  primer
aaatccagcgtttacggttttc

SEQ ID NO: 30 - DNA, 19 bp, artificial sequence
  primer
catgcccaggcgactccgc

SEQ ID NO: 31 - DNA, 21 bp, artificial sequence
  primer
tgtatgcgtcaacgtgaaacc

SEQ ID NO: 32 - DNA, 18 bp, artificial sequence
  primer
cgcttccagctccatcca

SEQ ID NO: 33 - DNA, 24 bp, artificial sequence
  primer
caccggaagtgcgtgaattctggg

SEQ ID NO: 34 - protein, 8 aa, Artificial Sequence
  synthetic construct - Caspase 3 cleavage site
LESGDEVD

SEQ ID NO: 35 - protein, 12 aa, artificial sequence
  Synthetic construct
RVPNKTVTVDGA

SEQ ID NO: 36 - protein, 12 aa, artificial sequence
  Synthetic construct
DRHKSKYSSTKS

SEQ ID NO: 37 - protein, 12 aa, artificial sequence
  Synthetic construct
KNFPQQKEFPLS

SEQ ID NO: 38 - protein, 12 aa, artificial sequence
  Synthetic construct
QRNSPPAMSRRD

SEQ ID NO: 39 - protein, 12 aa, artificial sequence
  Synthetic construct
TRKPNMPHGQYL

SEQ ID NO: 40 - protein, 12 aa, artificial sequence
  Synthetic construct
KPPHLAKLPFTT

SEQ ID NO: 41 - protein, 12 aa, artificial sequence
  Synthetic construct
NKRPPTSHRIHA

SEQ ID NO: 42 - protein, 12 aa, artificial sequence
  Synthetic construct
NLPRYQPPCKPL

SEQ ID NO: 43 - protein, 12 aa, artificial sequence
  Synthetic construct
RPPWKKPIPPSE

SEQ ID NO: 44 - protein, 12 aa, artificial sequence
  Synthetic construct
RQRPKDHFFSRP

SEQ ID NO: 45 - protein, 12 aa, artificial sequence
  Synthetic construct
SVPNKXVTVDGX

SEQ ID NO: 46 - protein, 12 aa, artificial sequence
  Synthetic construct
TTKWRHRAPVSP

SEQ ID NO: 47 - protein, 12 aa, artificial sequence
  Synthetic construct
WLGKNRIKPRAS

SEQ ID NO: 48 - protein, 12 aa, artificial sequence
  Synthetic construct
SNFKTPLPLTQS

SEQ ID NO: 49 - protein, 12 aa, artificial sequence
  Synthetic construct
SVSVGMKPSPRP

SEQ ID NO: 50 - protein, 7 aa, artificial sequence
  Synthetic construct
DLHTVYH

SEQ ID NO: 51 - protein, 7 aa, artificial sequence
  Synthetic construct
HIKPPTR

SEQ ID NO: 52 - protein, 7 aa, artificial sequence
  Synthetic construct
HPVWPAI

SEQ ID NO: 53 - protein, 7 aa, artificial sequence
  Synthetic construct
MPLYYLQ

SEQ ID NO: 54 - protein, 26 aa, artificial sequence
  Synthetic construct
HLTVPWRGGGSAVPFYSHSQITLPNH

SEQ ID NO: 55 - protein, 41 aa, artificial sequence
  Synthetic construct
GPHDTSSGGVRPNLHHTSKKEKRENRKVPFYSHSVTSRGNV

SEQ ID NO: 56 - protein, 7 aa, artificial sequence
  Synthetic construct
KHPTYRQ

SEQ ID NO: 57 - protein, 7 aa, artificial sequence
  Synthetic construct
HPMSAPR

SEQ ID NO: 58 - protein, 7 aa, artificial sequence
  Synthetic construct
MPKYYLQ

SEQ ID NO: 59 - protein, 7 aa, artificial sequence
  Synthetic construct
MHAHSIA

SEQ ID NO: 60 - protein, 12 aa, artificial sequence
  Synthetic construct
AKPISQHLQRGS

SEQ ID NO: 61 - protein, 12 aa, artificial sequence
  Synthetic construct
APPTPAAASATT

SEQ ID NO: 62 - protein, 12 aa, artificial sequence
  Synthetic construct
DPTEGARRTIMT

SEQ ID NO: 63 - protein, 12 aa, artificial sequence
  Synthetic construct
LDTSFPPVPFHA

SEQ ID NO: 64 - protein, 12 aa, artificial sequence
  Synthetic construct
LDTSFHQVPFHQ

SEQ ID NO: 65 - protein, 11 aa, artificial sequence
  Synthetic construct
LPRIANTWSPS

SEQ ID NO: 66 - protein, 12 aa, artificial sequence
  Synthetic construct
RTNAADHPAAVT

SEQ ID NO: 67 - protein, 12 aa, artificial sequence
  Synthetic construct
SLNWVTIPGPKI

SEQ ID NO: 68 - protein, 12 aa, artificial sequence
  Synthetic construct
TDMQAPTKSYSN

SEQ ID NO: 69 - protein, 12 aa, artificial sequence
  Synthetic construct
TIMTKSPSLSCG

SEQ ID NO: 70 - protein, 12 aa, artificial sequence
  Synthetic construct
TPALDGLRQPLR

SEQ ID NO: 71 - protein, 12 aa, artificial sequence
  Synthetic construct
TYPASRLPLLAP

SEQ ID NO: 72 - protein, 12 aa, artificial sequence
  Synthetic construct
AKTHKHPAPSYS

SEQ ID NO: 73 - protein, 12 aa, artificial sequence
  Synthetic construct
TDPTPFSISPER

SEQ ID NO: 74 - protein, 12 aa, artificial sequence
  Synthetic construct
SQNWQDSTSYSN

SEQ ID NO: 75 - protein, 12 aa, artificial sequence
  Synthetic construct
WHDKPQNSSKST

SEQ ID NO: 76 - protein, 12 aa, artificial sequence
  Synthetic construct
LDVESYKGTSMP

SEQ ID NO: 77 - protein, 7 aa, artificial sequence
  Synthetic construct
NTPKENW

SEQ ID NO: 78 - protein, 7 aa, artificial sequence
  Synthetic construct
NTPASNR

SEQ ID NO: 79 - protein, 7 aa, artificial sequence
  Synthetic construct
PRGMLST

SEQ ID NO: 80 - protein, 7 aa, artificial sequence
  Synthetic construct
PPTYLST

SEQ ID NO: 81 - protein, 12 aa, artificial sequence
  Synthetic construct
TIPTHRQHDYRS

SEQ ID NO: 82 - protein, 7 aa, artificial sequence
  Synthetic construct
TPPTHRL

SEQ ID NO: 83 - protein, 7 aa, artificial sequence
  Synthetic construct
LPTMSTP

SEQ ID NO: 84 - protein, 7 aa, artificial sequence
  Synthetic construct
LGTNSTP

SEQ ID NO: 85 - protein, 12 aa, artificial sequence
  Synthetic construct
TPLTGSTNLLSS

SEQ ID NO: 86 - protein, 7 aa, artificial sequence
  Synthetic construct
TPLTKET

SEQ ID NO: 87 - protein, 7 aa, artificial sequence
  Synthetic construct
KQSHNPP

SEQ ID NO: 88 - protein, 7 aa, artificial sequence
  Synthetic construct
QQSHNPP

SEQ ID NO: 89 - protein, 7 aa, artificial sequence
  Synthetic construct
TQPHNPP

SEQ ID NO: 90 - protein, 12 aa, artificial sequence
  Synthetic construct
STNLLRTSTVHP

SEQ ID NO: 91 - protein, 12 aa, artificial sequence
  Synthetic construct
HTQPSYSSTNLF

SEQ ID NO: 92 - protein, 7 aa, artificial sequence
  Synthetic construct
SLLSSHA

SEQ ID NO: 93 - protein, 12 aa, artificial sequence
  Synthetic construct
QQSSISLSSHAV

SEQ ID NO: 94 - protein, 7 aa, artificial sequence
  Synthetic construct
NASPSSL

SEQ ID NO: 95 - protein, 7 aa, artificial sequence
  Synthetic construct
HSPSSLR

SEQ ID NO: 96 - protein, 7 aa, artificial sequence
  Synthetic construct
KXSHHTH

SEQ ID NO: 97 - protein, 7 aa, artificial sequence
  Synthetic construct
EXSHHTH

SEQ ID NO: 98 - protein, 12 aa, artificial sequence
  Synthetic construct
SHHTHYGQPGPV

SEQ ID NO: 99 - protein, 7 aa, artificial sequence
  Synthetic construct
LESTSLL

SEQ ID NO: 100 - protein, 7 aa, artificial sequence
  Synthetic construct
DLTLPFH

SEQ ID NO: 101 - protein, 8 aa, artificial sequence
  Synthetic construct
RTNAADHP

SEQ ID NO: 102 - protein, 12 aa, artificial sequence
  Synthetic construct
IPWWNIRAPLNA

SEQ ID NO: 103 - protein, 18 aa, artificial sequence
  Synthetic construct
EQISGSLVAAPWEGEGER

SEQ ID NO: 104 - protein, 12 aa, artificial sequence
  synthetic hair-binding peptide
TPPELLHGAPRS

SEQ ID NO: 105 - protein, 18 aa, artificial sequence
  Synthetic construct
LDTSFHQVPFHQKRKRKD

SEQ ID NO: 106 - protein, 18 aa, artificial sequence
  Synthetic construct
EQISGSLVAAPWKRKRKD

SEQ ID NO: 107 - protein, 18 aa, artificial sequence
  Synthetic construct
TPPELLHGDPRSKRKRKD

SEQ ID NO: 108 - protein, 13 aa, artificial sequence
  Synthetic construct
NTSQLSTEGEGED

SEQ ID NO: 109 - protein, 13 aa, artificial sequence
  Synthetic construct
TPPELLHGDPRSC

SEQ ID NO: 110 - protein, 20 aa, artificial sequence
  synthetic hair-binding peptide
HINKTNPHQGNHHSEKTQRQ

SEQ ID NO: 111 - protein, 15 aa, artificial sequence
  Synthetic construct
HAHKNQKETHQRHAA

SEQ ID NO: 112 - protein, 15 aa, artificial sequence
  Synthetic construct
HEHKNQKETHQRHAA

SEQ ID NO: 113 - protein, 20 aa, artificial sequence
  Synthetic construct
HNHMQERYTEPQHSPSVNGL

SEQ ID NO: 114 - protein, 17 aa, artificial sequence
  Synthetic construct
THSTHNHGSPRHTNADA

SEQ ID NO: 115 - protein, 20 aa, artificial sequence
  synthetic hair-binding peptide
GSCVDTHKADSCVANNGPAT

SEQ ID NO: 116 - protein, 20 aa, artificial sequence
  synthetic hair-binding peptide
AQSQLPDKHSGLHERAPQRY

SEQ ID NO: 117 - protein, 20 aa, artificial sequence
  Synthetic construct
AQSQLPAKHSGLHERAPQRY

SEQ ID NO: 118 - protein, 20 aa, artificial sequence
  Synthetic construct
AQSQLPEKHSGLHERAPQRY

SEQ ID NO: 119 - protein, 20 aa, artificial sequence
  synthetic hair-binding peptide
TDMMHNHSDNSPPHRRSPRN

SEQ ID NO: 120 - protein, 20 aa, artificial sequence
  synthetic hair-binding peptide
TPPELAHTPHHLAQTRLTDR

SEQ ID NO: 121 - protein, 12 aa, artificial sequence
  Synthetic construct
RLLRLLRLLRLL

SEQ ID NO: 122 - protein, 12 aa, artificial sequence
  Synthetic construct
TPPELLHGEPRS

SEQ ID NO: 123 - protein, 12 aa, artificial sequence
  Synthetic construct
TPPELLHGAPRS

SEQ ID NO: 124 - protein, 12 aa, artificial sequence
  Synthetic construct
EQISGSLVAAPW

SEQ ID NO: 125 - protein, 12 aa, artificial sequence
  Synthetic construct
NEVPARNAPWLV

SEQ ID NO: 126 - protein, 13 aa, artificial sequence
  Synthetic construct
NSPGYQADSVAIG

SEQ ID NO: 127 - protein, 12 aa, artificial sequence
  Synthetic construct
AKPISQHLQRGS

SEQ ID NO: 128 - protein, 12 aa, artificial sequence
  Synthetic construct
LDTSFPPVPFHA

SEQ ID NO: 129 - protein, 12 aa, artificial sequence
  Synthetic construct
SLNWVTIPGPKI

SEQ ID NO: 130 - protein, 12 aa, artificial sequence
  Synthetic construct
TQDSAQKSPSPL

SEQ ID NO: 131 - protein, 12 aa, artificial sequence
  Synthetic construct
KELQTRNVVQRE

SEQ ID NO: 132 - protein, 12 aa, artificial sequence
  Synthetic construct
QRNSPPAMSRRD

SEQ ID NO: 133 - protein, 12 aa, artificial sequence
  Synthetic construct
TPTANQFTQSVP

SEQ ID NO: 134 - protein, 12 aa, artificial sequence
  Synthetic construct
AAGLSQKHERNR

SEQ ID NO: 135 - protein, 12 aa, artificial sequence
  Synthetic construct
ETVHQTPLSDRP

SEQ ID NO: 136 - protein, 12 aa, artificial sequence
  Synthetic construct
KNFPQQKEFPLS

SEQ ID NO: 137 - protein, 12 aa, artificial sequence
  Synthetic construct
LPALHIQRHPRM

SEQ ID NO: 138 - protein, 12 aa, artificial sequence
  Synthetic construct
QPSHSQSHNLRS

SEQ ID NO: 139 - protein, 12 aa, artificial sequence
  Synthetic construct
RGSQKSKPPRPP

SEQ ID NO: 140 - protein, 12 aa, artificial sequence
  Synthetic construct
THTQKTPLLYYH

SEQ ID NO: 141 - protein, 12 aa, artificial sequence
  Synthetic construct
TKGSSQAILKST

SEQ ID NO: 142 - protein, 7 aa, artificial sequence
  Synthetic construct
TAATTSP

SEQ ID NO: 143 - protein, 7 aa, artificial sequence
  Synthetic construct
LGIPQNL

SEQ ID NO: 144 - protein, 20 aa, artificial sequence
  Synthetic construct
THSTHNHGSPRHTNADAGNP

SEQ ID NO: 145 - protein, 20 aa, artificial sequence
  Synthetic construct
QQHKVHHQNPDRSTQDAHHS

SEQ ID NO: 146 - protein, 15 aa, artificial sequence
  Synthetic construct
HHGTHHNATKQKNHV

SEQ ID NO: 147 - protein, 15 aa, artificial sequence
  Synthetic construct
STLHKYKSQDPTPHH

SEQ ID NO: 148 - protein, 12 aa, artificial sequence
  Synthetic construct
SVSVGMKPSPRP

SEQ ID NO: 149 - protein, 12 aa, artificial sequence
  synthetic construct
TPPTNVLMLATK

SEQ ID NO: 150 - protein, 12 aa, artificial sequence
  Synthetic construct
TPPELLHGDPRS

SEQ ID NO: 151 - protein, 7 aa, artificial sequence
  synthetic construct
NTSQLST

SEQ ID NO: 152 - protein, 15 aa, artificial sequence
  Synthetic construct
STLHKYKSQDPTPHH

SEQ ID NO: 153 - protein, 12 aa, artificial sequence
  synthetic construct
GMPAMHWIHPFA

SEQ ID NO: 154 - protein, 15 aa, artificial sequence
  synthetic construct
HDHKNQKETHQRHAA

SEQ ID NO: 155 - protein, 20 aa, artificial sequence
  Synthetic construct
HNHMQERYTDPQHSPSVNGL

SEQ ID NO: 156 - protein, 20 aa, artificial sequence
  synthetic construct
TAEIQSSKNPNPHPQRSWTN

SEQ ID NO: 157 - protein, 21 aa, artificial sequence
  synthetic construct
SSADFASFGFFGFSAASADSR

SEQ ID NO: 158 - protein, 23 aa, artificial sequence
  synthetic construct
SSFAEAWSRAWPRAEVFFPSRGY

SEQ ID NO: 159 - protein, 17 aa, artificial sequence
  synthetic construct
SSFSVNEPHAWMAPLSR

SEQ ID NO: 160 - protein, 17 aa, artificial sequence
  synthetic construct
SSFSWVYGHGGLGFASR

SEQ ID NO: 161 - protein, 17 aa, artificial sequence
  synthetic construct
SSFVSWSPYKSPPELSR

SEQ ID NO: 162 - protein, 21 aa, artificial sequence
  synthetic construct
SSFYGSSAFVSSGVSVAYGSR

SEQ ID NO: 163 - protein, 21 aa, artificial sequence
  synthetic construct
SSGSVAVSAEASWFSGVAASR

SEQ ID NO: 164 - protein, 15 aa, artificial sequence
  synthetic construct
SSHDEHYQYHYYSSR

SEQ ID NO: 165 - protein, 15 aa, artificial sequence
  synthetic construct
SSHYYYNDYDHQSSR

SEQ ID NO: 166 - protein, 17 aa, artificial sequence
  synthetic construct
SSLFNMYGHQSVLGPSR

SEQ ID NO: 167 - protein, 17 aa, artificial sequence
  synthetic construct
SSLFSDVHYGSNKALSR

SEQ ID NO: 168 - protein, 17 aa, artificial sequence
  synthetic construct
SSLLSDFHYGDMWDASR

SEQ ID NO: 169 - protein, 15 aa, artificial sequence
  synthetic construct
SSNYNYNYNYQYSSR

SEQ ID NO: 170 - protein, 21 aa, artificial sequence
  synthetic construct
SSNYNYNYNYQYSSREGEGER

SEQ ID NO: 171 - protein, 21 aa, ARTIFICIAL SEQUENCE
  Synthetic construct
SSNYNYNYNYQYSSRKRKRKD

SEQ ID NO: 172 - protein, 15 aa, artificial sequence
  synthetic construct
SSQYYQDYQYYHSSR

SEQ ID NO: 173 - protein, 23 aa, artificial sequence
  synthetic construct
SSSCMGSHNPRMSVEESTRNCSR

SEQ ID NO: 174 - protein, 23 aa, artificial sequence
  synthetic construct
SSSCNNNWFYSSTLPGGDHACSR

SEQ ID NO: 175 - protein, 23 aa, artificial sequence
  synthetic construct
SSSCYDVECSSFVAWMRGPSSSR

SEQ ID NO: 176 - protein, 21 aa, artificial sequence
  synthetic construct
SSSFAASSAFSFLVDAVAWSR

SEQ ID NO: 177 - protein, 17 aa, artificial sequence
  synthetic construct
SSSFAYLVPDDGWLSSR

SEQ ID NO: 178 - protein, 21 aa, artificial sequence
  synthetic construct
SSSGAVFSSGGADAGWGVWSR

SEQ ID NO: 179 - protein, 23 aa, artificial sequence
  synthetic construct
SSSSADAAYGHCCGAGFSTFSSR

SEQ ID NO: 180 - protein, 23 aa, artificial sequence
  synthetic construct
SSSSDVHNSIIGWDFYHSRGSSR

SEQ ID NO: 181 - protein, 21 aa, artificial sequence
  synthetic construct
SSSSLDFFSYSAFSGGVAESR

SEQ ID NO: 182 - protein, 23 aa, artificial sequence
  synthetic construct
SSSSNDSNVSWFHYYASGLTSSR

SEQ ID NO: 183 - protein, 21 aa, artificial sequence
  synthetic construct
SSVDYEVPLAVAAEWGFSVSR

SEQ ID NO: 184 - protein, 15 aa, artificial sequence
  synthetic construct
SSYHYDYDHYYESSR

SEQ ID NO: 185 - protein, 15 aa, artificial sequence
  synthetic construct
SSYYNYHYQYQDSSR

SEQ ID NO: 186 - protein, 15 aa, artificial sequence
  synthetic construct
SSYYYDYYQQDYSSR

SEQ ID NO: 187 - protein, 12 aa, artificial sequence
  Synthetic construct
KRGRHKRPKRHK

SEQ ID NO: 188 - protein, 7 aa, artificial sequence
  Synthetic construct
RLLRLLR

SEQ ID NO: 189 - protein, 12 aa, artificial sequence
  Synthetic construct
HKPRGGRKKALH

SEQ ID NO: 190 - protein, 18 aa, artificial sequence
  Synthetic construct
KPRPPHGKKHRPKHRPKK

SEQ ID NO: 191 - protein, 18 aa, artificial sequence
  Synthetic construct
RGRPKKGHGKRPGHRARK

SEQ ID NO: 192 - protein, 12 aa, artificial sequence
  Synthetic construct
TPFHSPENAPGS

SEQ ID NO: 193 - protein, 13 aa, artificial sequence
  Synthetic construct
TPFHSPENAPGSK

SEQ ID NO: 194 - protein, 16 aa, artificial sequence
  Synthetic construct
TPFHSPENAPGSGGGS

SEQ ID NO: 195 - protein, 17 aa, artificial sequence
  Synthetic construct
TPFHSPENAPGSGGGSS

SEQ ID NO: 196 - protein, 15 aa, artificial sequence
  Synthetic construct
TPFHSPENAPGSGGG

SEQ ID NO: 197 - protein, 7 aa, artificial sequence
  Synthetic construct
FTQSLPR

SEQ ID NO: 198 - protein, 12 aa, artificial sequence
  Synthetic construct
KQATFPPNPTAY

SEQ ID NO: 199 - protein, 12 aa, artificial sequence
  Synthetic construct
HGHMVSTSQLSI

SEQ ID NO: 200 - protein, 7 aa, artificial sequence
  Synthetic construct
LSPSRMK

SEQ ID NO: 201 - protein, 7 aa, artificial sequence
  Synthetic construct
LPIPRMK

SEQ ID NO: 202 - protein, 7 aa, artificial sequence
  Synthetic construct
HQRPYLT

SEQ ID NO: 203 - protein, 7 aa, artificial sequence
  Synthetic construct
FPPLLRL

SEQ ID NO: 204 - protein, 7 aa, artificial sequence
  Synthetic construct
QATFMYN

SEQ ID NO: 205 - protein, 11 aa, artificial sequence
  Synthetic construct
VLTSQLPNHSM

SEQ ID NO: 206 - protein, 7 aa, artificial sequence
  Synthetic construct
HSTAYLT

SEQ ID NO: 207 - protein, 12 aa, artificial sequence
  Synthetic construct
APQQRPMKTFNT

SEQ ID NO: 208 - protein, 12 aa, artificial sequence
  Synthetic construct
APQQRPMKTVQY

SEQ ID NO: 209 - protein, 7 aa, artificial sequence
  Synthetic construct
PPWLDLL

SEQ ID NO: 210 - protein, 7 aa, artificial sequence
  Synthetic construct
PPWTFPL

SEQ ID NO: 211 - protein, 7 aa, artificial sequence
  Synthetic construct
SVTHLTS

SEQ ID NO: 212 - protein, 7 aa, artificial sequence
  Synthetic construct
VITRLTS

SEQ ID NO: 213 - protein, 12 aa, artificial sequence
  Synthetic construct
DLKPPLLALSKV

SEQ ID NO: 214 - protein, 12 aa, artificial sequence
  Synthetic construct
SHPSGALQEGTF

SEQ ID NO: 215 - protein, 12 aa, artificial sequence
  Synthetic construct
FPLTSKPSGACT

SEQ ID NO: 216 - protein, 12 aa, artificial sequence
  Synthetic construct
DLKPPLLALSKV

SEQ ID NO: 217 - protein, 7 aa, artificial sequence
  Synthetic construct
PLLALHS

SEQ ID NO: 218 - protein, 7 aa, artificial sequence
  Synthetic construct
VPISTQI

SEQ ID NO: 219 - protein, 12 aa, artificial sequence
  Synthetic construct
YAKQHYPISTFK

SEQ ID NO: 220 - protein, 7 aa, artificial sequence
  Synthetic construct
HSTAYLT

SEQ ID NO: 221 - protein, 12 aa, artificial sequence
  Synthetic construct
STAYLVAMSAAP

SEQ ID NO: 222 - protein, 12 aa, artificial sequence
  Synthetic construct
SVSVGMKPSPRP

SEQ ID NO: 223 - protein, 12 aa, artificial sequence
  Synthetic construct
TMGFTAPRFPHY

SEQ ID NO: 224 - protein, 12 aa, artificial sequence
  Synthetic construct
NLQHSVGTSPVW

SEQ ID NO: 225 - protein, 15 aa, artificial sequence
  Synthetic construct
QLSYHAYPQANHHAP

SEQ ID NO: 226 - protein, 12 aa, artificial sequence
  Synthetic construct
NQAASITKRVPY

SEQ ID NO: 227 - protein, 14 aa, artificial sequence
  Synthetic construct
SGCHLVYDNGFCDH

SEQ ID NO: 228 - protein, 14 aa, artificial sequence
  Synthetic construct
ASCPSASHADPCAH

SEQ ID NO: 229 - protein, 14 aa, artificial sequence
  Synthetic construct
NLCDSARDSPRCKV

SEQ ID NO: 230 - protein, 12 aa, artificial sequence
  Synthetic construct
NHSNWKTAADFL

SEQ ID NO: 231 - protein, 12 aa, artificial sequence
  Synthetic construct
GSSTVGRPLSYE

SEQ ID NO: 232 - protein, 12 aa, artificial sequence
  Synthetic construct
SDTISRLHVSMT

SEQ ID NO: 233 - protein, 12 aa, artificial sequence
  Synthetic construct
SPLTVPYERKLL

SEQ ID NO: 234 - protein, 12 aa, artificial sequence
  Synthetic construct
SPYPSWSTPAGR

SEQ ID NO: 235 - protein, 12 aa, artificial sequence
  Synthetic construct
VQPITNTRYEGG

SEQ ID NO: 236 - protein, 12 aa, artificial sequence
  Synthetic construct
WPMHPEKGSRWS

SEQ ID NO: 237 - protein, 14 aa, artificial sequence
  Synthetic construct
DACSGNGHPNNCDR

SEQ ID NO: 238 - protein, 14 aa, artificial sequence
  Synthetic construct
DHCLGRQLQPVCYP

SEQ ID NO: 239 - protein, 14 aa, artificial sequence
  Synthetic construct
DWCDTIIPGRTCHG

SEQ ID NO: 240 - protein, 12 aa, artificial sequence
  Synthetic construct
ALPRIANTWSPS

SEQ ID NO: 241 - protein, 12 aa, artificial sequence
  Synthetic construct
YPSFSPTYRPAF

SEQ ID NO: 242 - protein, 20 aa, artificial sequence
  Synthetic construct
AHPESLGIKYALDGNSDPHA

SEQ ID NO: 243 - protein, 20 aa, artificial sequence
  Synthetic construct
ASVSNYPPIHHLATSNTTVN

SEQ ID NO: 244 - protein, 14 aa, artificial sequence
  Synthetic construct
DECMEPLNAAHCWR

SEQ ID NO: 245 - protein, 14 aa, artificial sequence
  Synthetic construct
DECMHGSDVEFCTS

SEQ ID NO: 246 - protein, 14 aa, artificial sequence
  Synthetic construct
DLCSMQMMNTGCHY

SEQ ID NO: 247 - protein, 14 aa, artificial sequence
  Synthetic construct
DLCSSPSTWGSCIR

SEQ ID NO: 248 - protein, 20 aa, artificial sequence
  Synthetic construct
DPNESNYENATTVSQPTRHL

SEQ ID NO: 249 - protein, 20 aa, artificial sequence
  Synthetic construct
EPTHPTMRAQMHQSLRSSSP

SEQ ID NO: 250 - protein, 20 aa, artificial sequence
  Synthetic construct
GNTDTTPPNAVMEPTVQHKW

SEQ ID NO: 251 - protein, 15 aa, artificial sequence
  Synthetic construct
NGPDMVQSVGKHKNS

SEQ ID NO: 252 - protein, 15 aa, artificial sequence
  Synthetic construct
NGPEVRQIPANFEKL

SEQ ID NO: 253 - protein, 20 aa, artificial sequence
  Synthetic construct
NNTSADNPPETDSKHHLSMS

SEQ ID NO: 254 - protein, 20 aa, artificial sequence
  Synthetic construct
NNTWPEGAGHTMPSTNIRQA

SEQ ID NO: 255 - protein, 20 aa, artificial sequence
  Synthetic construct
NPTATPHMKDPMHSNAHSSA

SEQ ID NO: 256 - protein, 20 aa, artificial sequence
  Synthetic construct
NPTDHIPANSTNSRVSKGNT

SEQ ID NO: 257 - protein, 15 aa, artificial sequence
  Synthetic construct
NPTDSTHMMHARNHE

SEQ ID NO: 258 - protein, 14 aa, artificial sequence
  Synthetic construct
QHCITERLHPPCTK

SEQ ID NO: 259 - protein, 14 aa, artificial sequence
  Synthetic construct
TPCAPASFNPHCSR

SEQ ID NO: 260 - protein, 14 aa, artificial sequence
  Synthetic construct
TPCATYPHFSGCRA

SEQ ID NO: 261 - protein, 20 aa, artificial sequence
  Synthetic construct
WCTDFCTRSTPTSTSRSTTS

SEQ ID NO: 262 - protein, 20 aa, artificial sequence
  Synthetic construct
APPLKTYMQERELTMSQNKD

SEQ ID NO: 263 - protein, 20 aa, artificial sequence
  Synthetic construct
EPPTRTRVNNHTVTVQAQQH

SEQ ID NO: 264 - protein, 14 aa, artificial sequence
  Synthetic construct
GYCLRGDEPAVCSG

SEQ ID NO: 265 - protein, 20 aa, artificial sequence
  Synthetic construct
LSSKDFGVTNTDQRTYDYTT

SEQ ID NO: 266 - protein, 14 aa, artificial sequence
  Synthetic construct
NFCETQLDLSVCTV

SEQ ID NO: 267 - protein, 14 aa, artificial sequence
  Synthetic construct
NTCQPTKNATPCSA

SEQ ID NO: 268 - protein, 20 aa, artificial sequence
  Synthetic construct
PSEPERRDRNIAANAGRFNT

SEQ ID NO: 269 - protein, 18 aa, artificial sequence
  Synthetic construct
THNMSHFPPSGHPKRTAT

SEQ ID NO: 270 - protein, 14 aa, artificial sequence
  Synthetic construct
TTCPTMGTYHVCWL

SEQ ID NO: 271 - protein, 20 aa, artificial sequence
  Synthetic construct
YCADHTPDPANPNKICGYSH

SEQ ID NO: 272 - protein, 20 aa, artificial sequence
  Synthetic construct
AANPHTEWDRDAFQLAMPPK

SEQ ID NO: 273 - protein, 20 aa, artificial sequence
  Synthetic construct
DLHPMDPSNKRPDNPSDLHT

SEQ ID NO: 274 - protein, 14 aa, artificial sequence
  Synthetic construct
ESCVSNALMNQCIY

SEQ ID NO: 275 - protein, 20 aa, artificial sequence
  Synthetic construct
HNKADSWDPDLPPHAGMSLG

SEQ ID NO: 276 - protein, 20 aa, artificial sequence
  Synthetic construct
LNDQRKPGPPTMPTHSPAVG

SEQ ID NO: 277 - protein, 14 aa, artificial sequence
  Synthetic construct
NTCATSPNSYTCSN

SEQ ID NO: 278 - protein, 14 aa, artificial sequence
  Synthetic construct
SDCTAGLVPPLCAT

SEQ ID NO: 279 - protein, 20 aa, artificial sequence
  Synthetic construct
TIESSQHSRTHQQNYGSTKT

SEQ ID NO: 280 - protein, 20 aa, artificial sequence
  Synthetic construct
VGTMKQHPTTTQPPRVSATN

SEQ ID NO: 281 - protein, 20 aa, artificial sequence
  Synthetic construct
YSETPNDQKPNPHYKVSGTK

SEQ ID NO: 282 - protein, 12 aa, Artificial sequence
  synthetic construct
IPWWNIRAPLNA

SEQ ID NO: 283 - protein, 12 aa, Artificial Sequence
  synthetic construct
TAVMNVVNNQLS

SEQ ID NO: 284 - protein, 12 aa, Artificial Sequence
  synthetic construct
VPWWAPSKLSMQ

SEQ ID NO: 285 - protein, 12 aa, Artificial Sequence
  synthetic construct
MVMAPHTPRARS

SEQ ID NO: 286 - protein, 12 aa, Artificial Sequence
  synthetic construct
TYPNWAHLLSHY

SEQ ID NO: 287 - protein, 7 aa, Artificial Sequence
  synthetic construct
TPWWRIT

SEQ ID NO: 288 - protein, 7 aa, Artificial Sequence
  synthetic construct
DLTLPFH

SEQ ID NO: 289 - protein, 7 aa, Artificial Sequence
  synthetic construct
GTSIPAM

SEQ ID NO: 290 - protein, 7 aa, Artificial Sequence
  synthetic construct
HHKHVVA

SEQ ID NO: 291 - protein, 7 aa, Artificial Sequence
  synthetic construct
HHHKHFM

SEQ ID NO: 292 - protein, 7 aa, Artificial Sequence
  synthetic construct
HHHRHQG

SEQ ID NO: 293 - protein, 7 aa, Artificial Sequence
  synthetic construct
HHWHAPR

SEQ ID NO: 294 - protein, 12 aa, Artificial Sequence
  synthetic construct
APWHLSSQYSGT

SEQ ID NO: 295 - protein, 14 aa, Artificial Sequence
  synthetic construct
GYCLRVDEPTVCSG

SEQ ID NO: 296 - protein, 15 aa, Artificial Sequence
  synthetic construct
HIHPSDNFPHKNRTH

SEQ ID NO: 297 - protein, 20 aa, Artificial Sequence
  synthetic construct
HTHHDTHKPWPTDDHRNSSV

SEQ ID NO: 298 - protein, 20 aa, Artificial Sequence
  synthetic construct
PEDRPSRTNALHHNAHHHNA

SEQ ID NO: 299 - protein, 15 aa, Artificial Sequence
  synthetic construct
TPHNHATTNHHAGKK

SEQ ID NO: 300 - protein, 15 aa, Artificial Sequence
  synthetic construct
EMVKDSNQRNTRISS

SEQ ID NO: 301 - protein, 12 aa, Artificial Sequence
  synthetic construct
HYSRYNPGPHPL

SEQ ID NO: 302 - protein, 12 aa, Artificial Sequence
  synthetic construct
IDTFYMSTMSHS

SEQ ID NO: 303 - protein, 20 aa, Artificial Sequence
  synthetic construct
PMKEATHPVPPHKHSETPTA

SEQ ID NO: 304 - protein, 12 aa, Artificial Sequence
  synthetic construct
YQTSSPAKQSVG

SEQ ID NO: 305 - protein, 15 aa, Artificial Sequence
  Shampoo-resistant PMMA-binding peptide
HLPSYQITQTHAQYR

SEQ ID NO: 306 - protein, 20 aa, Artificial Sequence
  synthetic construct
TTPKTTYHQSRAPVTAMSEV

SEQ ID NO: 307 - protein, 15 aa, Artificial Sequence
  synthetic construct
DRIHHKSHHVTTNHF

SEQ ID NO: 308 - protein, 12 aa, Artificial Sequence
  synthetic construct
WAPEKDYMQLMK

SEQ ID NO: 309 - protein, 12 aa, Artificial sequence
  Polypropylene-binding peptides
TSDIKSRSPHHR

SEQ ID NO: 310 - protein, 12 aa, Artificial Sequence
  Polypropylenebinding peptide
HTQNMRMYEPWF

SEQ ID NO: 311 - protein, 7 aa, Artificial Sequence
  Polypropylene-binding peptide
LPPGSLA

SEQ ID NO: 312 - protein, 12 aa, Artificial Sequence
  Polypropylene-binding peptide
MPAVMSSAQVPR

SEQ ID NO: 313 - protein, 12 aa, Artificial Sequence
  Polypropylene-binding peptide
NQSFLPLDFPFR

SEQ ID NO: 314 - protein, 12 aa, Artificial Sequence
  Polypropylene-binding peptide
SILSTMSPHGAT

SEQ ID NO: 315 - protein, 12 aa, Artificial Sequence
  Polypropylenebinding peptide
SMKYSHSTAPAL

SEQ ID NO: 316 - protein, 12 aa, Artificial sequence
  Polytetrafluoroethylene-binding peptides
ESSYSWSPARLS

SEQ ID NO: 317 - protein, 12 aa, Artificial Sequence
  Polytetrafluoroethylene-binding peptide
GPLKLLHAWWQP

SEQ ID NO: 318 - protein, 7 aa, Artificial Sequence
  Polytetrafluoroethylene-binding peptide
NALTRPV

SEQ ID NO: 319 - protein, 7 aa, Artificial Sequence
  Polytetrafluoroethylene-binding peptide
SAPSSKN

SEQ ID NO: 320 - protein, 12 aa, Artificial Sequence
  Polytetrafluoroethylene-binding peptide
SVSVGMKPSPRP

SEQ ID NO: 321 - protein, 12 aa, Artificial Sequence
  Polytetrafluoroethylene-binding peptide
SYYSLPPIFHIP

SEQ ID NO: 322 - protein, 12 aa, Artificial Sequence
  Polytetrafluoroethylene-binding peptide
TFTPYSITHALL

SEQ ID NO: 323 - protein, 12 aa, Artificial Sequence
  Polytetrafluoroethylene-binding peptide
TMGFTAPRFPHY

SEQ ID NO: 324 - protein, 12 aa, Artificial Sequence
  Polytetrafluoroethylene-binding peptide
TNPFPPPPSSPA

SEQ ID NO: 325 - protein, 12 aa, Artificial sequence
  Polyethylene-binding peptides
HNKSSPLTAALP

SEQ ID NO: 326 - protein, 12 aa, Artificial Sequence
  Polyethylene-binding peptide
LPPWKHKTSGVA

SEQ ID NO: 327 - protein, 12 aa, Artificial Sequence
  Polyethylene-binding peptide
LPWWLRDSYLLP

SEQ ID NO: 328 - protein, 12 aa, Artificial Sequence
  Polyethylene-binding peptide
VPWWKHPPLPVP

SEQ ID NO: 329 - protein, 12 aa, Artificial Sequence
  Polyethylene-binding peptide
HHKQWHNHPHHA

SEQ ID NO: 330 - protein, 12 aa, Artificial Sequence
  Polyethylene-binding peptide
HIFSSWHQMWHR

SEQ ID NO: 331 - protein, 12 aa, Artificial Sequence
  Polyethylene-binding peptide
WPAWKTHPILRM

SEQ ID NO: 332 - protein, 7 aa, Artificial sequence
  Nylon-binding peptides
KTPPTRP

SEQ ID NO: 333 - protein, 7 aa, Artificial Sequence
  Nylon-binding peptide
VINPNLD

SEQ ID NO: 334 - protein, 7 aa, Artificial Sequence
  Nylon-binding peptide
KVWIVST

SEQ ID NO: 335 - protein, 7 aa, Artificial Sequence
  Nylon-binding peptide
AEPVAML

SEQ ID NO: 336 - protein, 7 aa, Artificial Sequence
  Nylon-binding peptide
AELVAML

SEQ ID NO: 337 - protein, 7 aa, Artificial Sequence
  Nylon-binding peptide
HSLRLDW

SEQ ID NO: 338 - protein, 13 aa, Artificial sequence
  Polystyrene-binding peptide
TSTASPTMQSKIR

SEQ ID NO: 339 - protein, 12 aa, Artificial Sequence
  Polystyrene-binding peptide
KRNHWQRMHLSA

SEQ ID NO: 340 - protein, 12 aa, Artificial Sequence
  Polystyrene-binding peptide
SHATPPQGLGPQ

SEQ ID NO: 341 - protein, 20 aa, artificial sequence
  synthetic construct
ATTPPSGKAAAHSAARQKGN

SEQ ID NO: 342 - protein, 15 aa, artificial sequence
  synthetic construct
DTIHPNKMKSPSSPL

SEQ ID NO: 343 - protein, 20 aa, artificial sequence
  synthetic construct
NGNNHTDIPNRSSYTGGSFA

SEQ ID NO: 344 - protein, 15 aa, ARTIFICIAL SEQUENCE
  synthetic construct
SDETGPQIPHRRPTW

SEQ ID NO: 345 - protein, 7 aa, Artificial Sequence
  synthetic construct
MPPPLMQ

SEQ ID NO: 346 - protein, 7 aa, Artificial Sequence
  Pigment-binding peptide
FHENWPS

SEQ ID NO: 347 - protein, 12 aa, Artificial Sequence
  Pigment-binding peptide
RTAPTTPLLLSL

SEQ ID NO: 348 - protein, 12 aa, Artificial Sequence
  Pigment-binding peptide
WHLSWSPVPLPT

SEQ ID NO: 349 - protein, 7 aa, Artificial Sequence
  Pigment-binding peptide
PHARLVG

SEQ ID NO: 350 - protein, 7 aa, Artificial Sequence
  Pigment-binding peptide
NIPYHHP

SEQ ID NO: 351 - protein, 7 aa, Artificial Sequence
  Pigment-binding peptide
TTMPAIP

SEQ ID NO: 352 - protein, 7 aa, Artificial Sequence
  Pigment-binding peptide
HNLPPRS

SEQ ID NO: 353 - protein, 12 aa, Artificial Sequence
  Pigment-binding peptide
AHKTQMGVRQPA

SEQ ID NO: 354 - protein, 12 aa, Artificial Sequence
  Pigment-binding peptide
ADNVQMGVSHTP

SEQ ID NO: 355 - protein, 12 aa, Artificial Sequence
  Pigment-binding peptide
AHNAQMGVSHPP

SEQ ID NO: 356 - protein, 12 aa, Artificial Sequence
  Pigment-binding peptide
ADYVGMGVSHRP

SEQ ID NO: 357 - protein, 12 aa, Artificial Sequence
  Pigment-binding peptide
SVSVGMKPSPRP

SEQ ID NO: 358 - protein, 7 aa, Artificial Sequence
  Pigment-binding peptide
YPNTALV

SEQ ID NO: 359 - protein, 7 aa, Artificial Sequence
  Pigment-binding peptide
VATRIVS

SEQ ID NO: 360 - protein, 12 aa, Artificial Sequence
  Pigment-binding peptide
HSLKNSMLTVMA

SEQ ID NO: 361 - protein, 7 aa, Artificial Sequence
  Pigment-binding peptide
NYPTQAP

SEQ ID NO: 362 - protein, 7 aa, Artificial Sequence
  Pigment-binding peptide
KCCYSVG

SEQ ID NO: 363 - protein, 12 aa, Artificial Sequence
  Pigment-binding peptide
RHDLNTWLPPVK

SEQ ID NO: 364 - protein, 12 aa, artificial sequence
  Synthetic construct
EISLPAKLPSAS

SEQ ID NO: 365 - protein, 12 aa, Artificial Sequence
  Pigment-binding peptide
SDYVGMRPSPRH

SEQ ID NO: 366 - protein, 12 aa, Artificial Sequence
  Pigment-binding peptide
SDYVGMRLSPSQ

SEQ ID NO: 367 - protein, 12 aa, Artificial Sequence
  Pigment-binding peptide
SVSVGIQPSPRP

SEQ ID NO: 368 - protein, 12 aa, Artificial Sequence
  Pigment-binding peptide
YVSVGIKPSPRP

SEQ ID NO: 369 - protein, 12 aa, Artificial Sequence
  Pigment-binding peptide
YVCEGIHPCPRP

SEQ ID NO: 370 - protein, 12 aa, artificial sequence
  synthetic pigment binding peptide
WAPEKDHMQLMK

SEQ ID NO: 371 - protein, 12 aa, artificial sequence
  Iron oxide-binding peptide
WAPEKDYMQLMK

SEQ ID NO: 372 - protein, 20 aa, artificial sequence
  Iron oxide-binding peptide
CPLDTPTHKTKHEYKTRCRH

SEQ ID NO: 373 - protein, 18 aa, artificial sequence
  Iron oxide-binding peptide
DHDHPRLHKRQEKSEHLH

SEQ ID NO: 374 - protein, 20 aa, artificial sequence
  Iron oxide-binding peptide
DSHHNHHKQDSRPQHRKTPN

SEQ ID NO: 375 - protein, 15 aa, artificial sequence
  Iron oxide-binding peptide
EGGNAPHHKPHHRKH

SEQ ID NO: 376 - protein, 18 aa, artificial sequence
  Iron oxide-binding peptide
HDSHRPLTQHGHRHSHVP

SEQ ID NO: 377 - protein, 18 aa, artificial sequence
  Iron oxide-binding peptide
HDSNHCSHSTRRPNCART

SEQ ID NO: 378 - protein, 15 aa, artificial sequence
  Iron oxide-binding peptide
ATRVDNTPASNPPSL

SEQ ID NO: 379 - protein, 15 aa, artificial sequence
  Iron oxide-binding peptide
DGIKPFHLMTPTLAN

SEQ ID NO: 380 - protein, 18 aa, artificial sequence
  Iron oxide-binding peptide
DITPPGSTHHRKPHRHQH

SEQ ID NO: 381 - protein, 15 aa, artificial sequence
  Iron oxide-binding peptide
DNLWPQPLNVEDDRY

SEQ ID NO: 382 - protein, 18 aa, artificial sequence
  Iron oxide-binding peptide
ENEKHRHNTHEALHSHFK

SEQ ID NO: 383 - protein, 20 aa, artificial sequence
  Iron oxide-binding peptide
GAIWPASSALMTEHNPTDNH

SEQ ID NO: 384 - protein, 15 aa, artificial sequence
  Iron oxide-binding peptide
GDTNQDTVMWYYTVN

SEQ ID NO: 385 - protein, 15 aa, artificial sequence
  Iron oxide-binding peptide
HNGPYGMLSTGKIHF

SEQ ID NO: 386 - protein, 15 aa, artificial sequence
  Iron oxide-binding peptide
LDGGYRDTPDNYLKG

SEQ ID NO: 387 - protein, 15 aa, artificial sequence
  Iron oxide-binding peptide
LHTKTENSHTNMKTT

SEQ ID NO: 388 - protein, 20 aa, artificial sequence
  Iron oxide-binding peptide
NAQYDPPTLNKGAVRKAAST

SEQ ID NO: 389 - protein, 15 aa, artificial sequence
  Iron oxide-binding peptide
NGNNHTDIPNRSSYT

SEQ ID NO: 390 - protein, 18 aa, artificial sequence
  Iron oxide-binding peptide
QSTNHHHPHAKHPRVNTH

SEQ ID NO: 391 - protein, 15 aa, artificial sequence
  Iron oxide-binding peptide
SNNDYVGTYPATAIQ

SEQ ID NO: 392 - protein, 15 aa, artificial sequence
  Iron oxide-binding peptide
STQHNLHDRNIYFVS

SEQ ID NO: 393 - protein, 20 aa, artificial sequence
  Iron oxide-binding peptide
TANNKTPAGAPNAAVGLAQR

SEQ ID NO: 394 - protein, 15 aa, artificial sequence
  Iron oxide-binding peptide
TEPTRISNYRSIPND

SEQ ID NO: 395 - protein, 18 aa, artificial sequence
  Iron oxide-binding peptide
THNPREHARHHHHNEYKH

SEQ ID NO: 396 - protein, 15 aa, artificial sequence
  Iron oxide-binding peptide
THPPCWYETNCIVQE

SEQ ID NO: 397 - protein, 20 aa, artificial sequence
  Iron oxide-binding peptide
TTNPHKPASHHHDHRPALRH

SEQ ID NO: 398 - protein, 15 aa, artificial sequence
  Iron oxide-binding peptide
WLVADNATDGHSHQK

SEQ ID NO: 399 - protein, 15 aa, artificial sequence
  Iron oxide-binding peptide
YTDSMSDQTPEFAKY

SEQ ID NO: 400 - protein, 7 aa, Artificial Sequence
  Print Media Binding Peptide
SILPYPY

SEQ ID NO: 401 - protein, 7 aa, Artificial Sequence
  Cotton Binding Peptide
STASYTR

SEQ ID NO: 402 - protein, 7 aa, Artificial Sequence
  Polyester/Cotton Binding Peptide
LPVRPWT

SEQ ID NO: 403 - protein, 7 aa, Artificial Sequence
  Paper Binding Peptide
GNTPSRA

SEQ ID NO: 404 - protein, 7 aa, Artificial Sequence
  Paper Binding Peptide
HAIYPRH

SEQ ID NO: 405 - protein, 7 aa, Artificial Sequence
  Paper Binding Peptide
YQDSAKT

SEQ ID NO: 406 - protein, 7 aa, Artificial Sequence
  Cellulose Binding Peptide
VPRVTSI

SEQ ID NO: 407 - protein, 7 aa, Artificial Sequence
  Cellulose Binding Peptide
MANHNLS

SEQ ID NO: 408 - protein, 7 aa, Artificial Sequence
  Pigment Binding and Cellulose Binding Peptide
FHENWPS

SEQ ID NO: 409 - protein, 12 aa, Artificial Sequence
  Cellulose Binding Peptide
THKTSTQRLLAA

SEQ ID NO: 410 - protein, 12 aa, Artificial Sequence
  Cellulose Binding Peptide
KCCYVNVGSVFS

SEQ ID NO: 411 - protein, 12 aa, Artificial Sequence
  Cellulose Binding Peptide
AHMQFRTSLTPH

SEQ ID NO: 412 - protein, 27 aa, artificial sequence
  Clay-binding peptide
GHGSPSNSHHGSKKCDMGNSRAKCKRL

SEQ ID NO: 413 - protein, 27 aa, artificial sequence
  Clay-binding peptide
SDRHNLRNSWSISRHCRRKQGRCLPAH

SEQ ID NO: 414 - protein, 27 aa, artificial sequence
  Clay-binding peptide
KKSNKGHHPSSKGKGPPWSEWDKKNGP

SEQ ID NO: 415 - protein, 27 aa, artificial sequence
  Clay-binding peptide
KKSNKGPHPSSKGKGPPWSEWDKKNGP

SEQ ID NO: 416 - protein, 22 aa, artificial sequence
  Clay-binding peptide
VGRHHSKAKQKRPHGGKGQNKN

SEQ ID NO: 417 - protein, 22 aa, artificial sequence
  Clay-binding peptide
VGRHHPKAKQKRPHGGKGQNKN

SEQ ID NO: 418 - protein, 17 aa, artificial sequence
  Clay-binding peptide
GRRPRARGRSRRGSTKT

SEQ ID NO: 419 - protein, 19 aa, artificial sequence
  Clay-binding peptide
LGVIRNHVVRGRRHHQHVR

SEQ ID NO: 420 - protein, 27 aa, artificial sequence
  Clay-binding peptide
QPGRPTEVHPELVRKSAYLVNPSEDIR

SEQ ID NO: 421 - protein, 27 aa, artificial sequence
  Clay-binding peptide
HRSEKPKNVKYKRGYWERGNQKKHGPG

SEQ ID NO: 422 - protein, 27 aa, artificial sequence
  Clay-binding peptide
GSHKRRGSYALLRTRGVGRQAELEHLL

SEQ ID NO: 423 - protein, 27 aa, artificial sequence
  Clay-binding peptide
VGEKPRRKSKGAKAKKARTKEEKLPKN

SEQ ID NO: 424 - protein, 27 aa, artificial sequence
  Clay-binding peptide
NKGHKQSGSPRHSNKKEKKTQQKRGQP

SEQ ID NO: 425 - protein, 27 aa, artificial sequence
  Clay-binding peptide
HWGSQHKTGLRNHKRSRRDSLGKRGTD

SEQ ID NO: 426 - protein, 27 aa, artificial sequence
  Clay-binding peptide
KGWGSSSGPPGLTGKALGKGRLKPKKK

SEQ ID NO: 427 - protein, 27 aa, artificial sequence
  Calcium carbonate binding peptide
RNNKGSKKVDDKRRKTVHNTKSRAKYS

SEQ ID NO: 428 - protein, 27 aa, artificial sequence
  Calcium carbonate binding peptide
RNNKGSKKVDDKRRKTVHNTKSRAKHS

SEQ ID NO: 429 - protein, 27 aa, artificial sequence
  Calcium carbonate binding peptide
RDNKGSKKVDDKRRKTVHNTKSRAKYS

SEQ ID NO: 430 - protein, 27 aa, artificial sequence
  Calcium carbonate binding peptide
RNNKGSKKVDDKRRKTVHSTKSRAKYS

SEQ ID NO: 431 - protein, 27 aa, artificial sequence
  Calcium carbonate binding peptide
RNNKGSRKVDDKRRKTVHNTKSRAKYS

SEQ ID NO: 432 - protein, 27 aa, artificial sequence
  Calcium carbonate binding peptide
RNNKGSKKADDKRRKTVHSTKSRAKYS

SEQ ID NO: 433 - protein, 27 aa, artificial sequence
  Calcium carbonate binding peptide
RNNKGSKKVDDKRRKAVHNKKSRAKYS

SEQ ID NO: 434 - protein, 27 aa, artificial sequence
  Calcium carbonate binding peptide
RNNKGSKKVDDKRRKTVHNTRSRAKYS

SEQ ID NO: 435 - protein, 27 aa, artificial sequence
  Calcium carbonate binding peptide
RNNKGSKKVDDKRRKTVHNTKSRAKFS

SEQ ID NO: 436 - protein, 27 aa, artificial sequence
  Calcium carbonate binding peptide
QRRKLRHPKEKWFGWSEKKVIKKWSRK

SEQ ID NO: 437 - protein, 27 aa, artificial sequence
  Calcium carbonate binding peptide
QRRKFRHPKEKWFGWSEKKVIKXNGRP

SEQ ID NO: 438 - protein, 27 aa, artificial sequence
  Calcium carbonate binding peptide
HKRLVQNKPHRTRKIEGWIKHMVKRQH

SEQ ID NO: 439 - protein, 27 aa, artificial sequence
  Calcium carbonate binding peptide
TRGHIMRPCWIGAMKQGVKKKRTPGWR

SEQ ID NO: 440 - protein, 27 aa, artificial sequence
  Calcium carbonate binding peptide
WKVKRRMVTRTYEFMGKKPCMMLTKRL

SEQ ID NO: 441 - protein, 26 aa, artificial sequence
  Calcium carbonate binding peptide
KKSNKGHHSKAKQKRPHGGKAQNKNT

SEQ ID NO: 442 - protein, 27 aa, artificial sequence
  Calcium carbonate binding peptide
RAHKERFVVRQIGRSQGYKTWQCVRVA

SEQ ID NO: 443 - protein, 27 aa, artificial sequence
  Calcium carbonate binding peptide
SQKPKGHKVKVVVKLCKRPYWRMLNTA

SEQ ID NO: 444 - protein, 27 aa, artificial sequence
  Calcium carbonate binding peptide
NHGCPVNWKVXNPPRGWQRLNHCKWWN

SEQ ID NO: 445 - protein, 27 aa, artificial sequence
  Calcium carbonate binding peptide
RNSRHKEWRRYKRTHVHSHEFYHVECW

SEQ ID NO: 446 - protein, 27 aa, artificial sequence
  Calcium carbonate binding peptide
HRSEKPKNVNYKRGYWERGNQKKHGPG

SEQ ID NO: 447 - protein, 27 aa, artificial sequence
  Calcium carbonate binding peptide
HERTRRGKPDRQKTTHEKRRQGLWIFM

SEQ ID NO: 448 - protein, 27 aa, artificial sequence
  Calcium carbonate binding peptide
PWGTNKRQKHKVHEAKALKKSLWYSNS

SEQ ID NO: 449 - protein, 27 aa, artificial sequence
  Calcium carbonate binding peptide
RRGVVLCHTHRNKRIRLAYSVTKKAWA

SEQ ID NO: 450 - protein, 27 aa, artificial sequence
  Calcium carbonate binding peptide
ERIRWRRLSAEIRAHKWSVLKFRLSCM

SEQ ID NO: 451 - protein, 27 aa, artificial sequence
  Calcium carbonate binding peptide
KTKEKKKEVKLHKKSLSLVLLADLWRL

SEQ ID NO: 452 - protein, 27 aa, artificial sequence
  Calcium carbonate binding peptide
LGKKHKQHSKVGHGKLSTRFLRRSKLF

SEQ ID NO: 453 - protein, 17 aa, artificial sequence
  Antimicrobial peptide
PKGLKKLLKGLKKLLKL

SEQ ID NO: 454 - protein, 16 aa, artificial sequence
  Antimicrobial peptide
KGLKKLLKGLKKLLKL

SEQ ID NO: 455 - protein, 16 aa, artificial sequence
  Antimicrobial peptide
KGLKKLLKLLKKLLKL

SEQ ID NO: 456 - protein, 14 aa, artificial sequence
  Antimicrobial peptide
LKKLLKLLKKLLKL

SEQ ID NO: 457 - protein, 12 aa, artificial sequence
  Antimicrobial peptide
LKKLLKLLKKLL

SEQ ID NO: 458 - protein, 17 aa, artificial sequence
  Antimicrobial peptide
VAKKLAKLAKKLAKLAL

SEQ ID NO: 459 - protein, 13 aa, artificial sequence
  Antimicrobial peptide
FAKLLAKALKKLL

SEQ ID NO: 460 - protein, 16 aa, artificial sequence
  Antimicrobial peptide
KGLKKGLKLLKKLLKL

SEQ ID NO: 461 - protein, 16 aa, artificial sequence
  Antimicrobial peptide
KGLKKLLKLGKKLLKL

SEQ ID NO: 462 - protein, 16 aa, artificial sequence
  Antimicrobial peptide
KGLKKLGKLLKKLLKL

SEQ ID NO: 463 - protein, 16 aa, artificial sequence
  Antimicrobial peptide
KGLKKLLKLLKKGLKL

SEQ ID NO: 464 - protein, 16 aa, artificial sequence
  Antimicrobial peptide
KGLKKLLKLLKKLGKL

SEQ ID NO: 465 - protein, 19 aa, artificial sequence
  Antimicrobial peptide
FALALKALKKLKKALKKAL

SEQ ID NO: 466 - protein, 17 aa, artificial sequence
  Antimicrobial peptide
FAKKLAKLAKKLAKLAL

SEQ ID NO: 467 - protein, 13 aa, artificial sequence
  Antimicrobial peptide
FAKLLAKLAKKLL

SEQ ID NO: 468 - protein, 15 aa, artificial sequence
  Antimicrobial peptide
FAKKLAKLALKLAKL

SEQ ID NO: 469 - protein, 10 aa, artificial sequence
  Antimicrobial peptide
FAKKLAKKLL

SEQ ID NO: 470 - protein, 13 aa, artificial sequence
  Antimicrobial peptide
FAKLLAKLAKKVL

SEQ ID NO: 471 - protein, 13 aa, artificial sequence
  Antimicrobial peptide
KYKKALKKLAKLL

SEQ ID NO: 472 - protein, 12 aa, artificial sequence
  Antimicrobial peptide
FALLKALLKKAL

SEQ ID NO: 473 - protein, 14 aa, artificial sequence
  Antimicrobial peptide
KRLFKKLKFSLRKY

SEQ ID NO: 474 - protein, 14 aa, artificial sequence
  Antimicrobial peptide
KRLFKKLLFSLRKY

SEQ ID NO: 475 - protein, 14 aa, artificial sequence
  Antimicrobial peptide
LLLFLLKKRKKRKY

SEQ ID NO: 476 - protein, 36 aa, Hyalophora cecropia
KWKLFKKIEKVGQNIRDGIIKAGPAVAWGQATQIAK

SEQ ID NO: 477 - protein, 23 aa, Xenopus laevis
GIGKFLHSAKKFGKAFVGEIMNS

SEQ ID NO: 478 - protein, 22 aa, Xenopus laevis
GIGKFLKKAKKFGKAFVKILKK

SEQ ID NO: 479 - protein, 12 aa, Bos taurus
RLCRIVVIRVCR

SEQ ID NO: 480 - protein, 13 aa, Bos taurus
ILPWKWPWWPWRR

SEQ ID NO: 481 - protein, 24 aa, Homo sapiens
DSHAKRHHGYKRKFHEKHHSHRGY